SEQUENCE LISTING (139 sequences)

SEQ ID NO: 1 - DNA, 23 bp, Artificial Sequence
  transcription enhancing RAP consensus
grhwdsatbmgrkmrvkdbagca

SEQ ID NO: 2 - DNA, 29 bp, Artificial Sequence
  RAP ID# 5713
gacaacatgagaacagttcagcagcacta

SEQ ID NO: 3 - DNA, 37 bp, Artificial Sequence
  RAP ID# 15488
cagcaatcaaccaatcccgaaagcatacagactggtg

SEQ ID NO: 4 - DNA, 41 bp, Artificial Sequence
  RAP ID# 14908
taccgtgacgtgccatccagtccatttcgccgtggtattgt

SEQ ID NO: 5 - DNA, 49 bp, Artificial Sequence
  RAP ID # 9885
gagcactaacagtaaaggagtcaatgatgtcaggagaacacgtttcatc

SEQ ID NO: 6 - DNA, 50 bp, Artificial Sequence
  RAP ID# 607
gctaagacgcagtgcaaaagtggtatcggaatgattcgtgtagccttgca

SEQ ID NO: 7 - DNA, 52 bp, Artificial Sequence
  RAP ID# 4962
gttgcagtgcggatgctgtgtgatggcgctatacaaaaaagttgtcgagtga

SEQ ID NO: 8 - DNA, 47 bp, Artificial Sequence
  RAP ID# 13404
cgactatgatcagaatcgggccatgttaaacaccctgaatgttggtc

SEQ ID NO: 9 - DNA, 50 bp, Artificial Sequence
  RAP ID# 15418CTAAACCAGGGTAGTCCGGCAGAGTAACGGTGACATCACGATAGCACCAT
ctaaaccagggtagtccggcagagtaacggtgacatcacgatagcaccat

SEQ ID NO: 10 - DNA, 46 bp, Artificial Sequence
  RAP ID# 602
attgagtcgaaatcggtattcaccggattgtacagattgagccatc

SEQ ID NO: 11 - DNA, 36 bp, Artificial Sequence
  RAP ID# 3439
aatgcgtgatttcttgtgctttcagtcccagttttg

SEQ ID NO: 12 - DNA, 44 bp, Artificial Sequence
  RAP ID# 3600
ggaatctgttggcgtggaataatcggtgagacgcatcatgttgt

SEQ ID NO: 13 - DNA, 41 bp, Artificial Sequence
  RAP ID# 5151
atcatcaggcgattgaagtgggtatggtgaactatctgtgg

SEQ ID NO: 14 - DNA, 36 bp, Artificial Sequence
  RAP ID# 14079
cttgggtttgttgattacggatattgagcttgtcat

SEQ ID NO: 15 - DNA, 24 bp, Artificial Sequence
  RAP ID# 14060
cacaaatcacacagcccatcaccc

SEQ ID NO: 16 - DNA, 31 bp, Artificial Sequence
  RAP ID# 14542
agtacaccggatcccagaaccaccagccgcc

SEQ ID NO: 17 - DNA, 37 bp, Artificial Sequence
  RAP ID# 11884
cagccaggtcacaatacccagcatcaacatgtggcat

SEQ ID NO: 18 - DNA, 49 bp, Artificial Sequence
  RAP ID# 3828
ccaaggtacccaagaagcgcccgcaggatcaaaaactgccagatatttt

SEQ ID NO: 19 - DNA, 35 bp, Artificial Sequence
  RAP ID# 2464
cagcatcacatgaccgatcgccatcaacaacgccc

SEQ ID NO: 20 - DNA, 28 bp, Artificial Sequence
  RAP ID# 1220
cagctaacccgatgatcaccgactgacc

SEQ ID NO: 21 - DNA, 24 bp, Artificial Sequence
  RAP ID# 7866
cagcatgcagcagatccagcgtgt

SEQ ID NO: 22 - DNA, 44 bp, Artificial Sequence
  RAP ID# 10966
agcaggtacattgtccacagacactgccagagcccactgtgtcc

SEQ ID NO: 23 - DNA, 46 bp, Artificial Sequence
  RAP ID# 4555
ccagagttaccaatacaggtggtacaaccgtatcccacaaggttaa

SEQ ID NO: 24 - DNA, 36 bp, Artificial Sequence
  RAP ID# 11531
tgatgatcagcacatcttccagagccatgtctttca

SEQ ID NO: 25 - DNA, 39 bp, Artificial Sequence
  RAP ID# 15
gtcacaatcatccctaataatgttcctccgcatcgtccc

SEQ ID NO: 26 - DNA, 51 bp, Artificial Sequence
  RAP ID# 683
ttaaacgaatgtctgcatatattgtggcgtattcgctttgccctgcatctg

SEQ ID NO: 27 - DNA, 55 bp, Artificial Sequence
  RAP ID# 10070
acctggttgacgaagctaaacgtctgctgaccacctgcaacatcccggttccgtc

SEQ ID NO: 28 - DNA, 48 bp, Artificial Sequence
  RAP ID# 15592
tacatcagccctgcaatcagcaatcccggcagcaacactccccagcca

SEQ ID NO: 29 - DNA, 53 bp, Artificial Sequence
  RAP ID# 5039
tcatgacgcagctgatgatccacattctttacccacacaaattcatgtccttt

SEQ ID NO: 30 - DNA, 46 bp, Artificial Sequence
  RAP ID# 2136
ccacttcaccatcgatccttcccgcattaaacaacatgtccgtttt

SEQ ID NO: 31 - DNA, 52 bp, Artificial Sequence
  RAP ID# 1800
acatgatcaccgcgctctattccgcttctgctgattccattcctattctgtg

SEQ ID NO: 32 - DNA, 43 bp, Artificial Sequence
  RAP ID# 3930
aacccttctgacgatcccattgctgcatcacaagccgtagttc

SEQ ID NO: 33 - DNA, 47 bp, Artificial Sequence
  RAP ID# 11599
tgccagatcagaacgcagcatttcctcaacatcttcaccaatgttca

SEQ ID NO: 34 - DNA, 53 bp, Artificial Sequence
  RAP ID# 15003
caagccaccacatcgcaaatgtgccaggagcgacctgttcttgttcaatttct

SEQ ID NO: 35 - DNA, 61 bp, Artificial Sequence
  RAP ID# 9533
cagcgccataaagccgatgatcatccactcaacgttgacgtagttgcagaacaccatcacc

SEQ ID NO: 36 - DNA, 51 bp, Artificial Sequence
  RAP ID# 803
ctgttccatgttaagcagatcccctgtcgaacccgttcaaagcactgcacc

SEQ ID NO: 37 - DNA, 43 bp, Artificial Sequence
  RAP ID# 9505
caatgtccatacttaatgtcgagaggatctccaccatctcaac

SEQ ID NO: 38 - DNA, 43 bp, Artificial Sequence
  RAP ID# 9559
ttcgttttcccaacctgaatctcgacaactccgtccacatcac

SEQ ID NO: 39 - DNA, 47 bp, Artificial Sequence
  RAP ID# 1510
tccaacccgtattcctcgaagtagtggatgaaagctatcgtcacaat

SEQ ID NO: 40 - DNA, 43 bp, Artificial Sequence
  RAP ID# 10243
taccaccaaaccaacgtacagagcgtacaagccaacattgccc

SEQ ID NO: 41 - DNA, 78 bp, Artificial Sequence
  RAP ID# 10822
cttcgacatccgcttcgtggtggaaatacccatccagcaggccgacaaggttggttttacgtactggatcttctttgc

SEQ ID NO: 42 - DNA, 74 bp, Artificial Sequence
  rut Rho termination sequence
cccttccttctccccatcgctacctcatatccgcacctcctcaaacgctacctcgaccagcctccctccctccc

SEQ ID NO: 43 - DNA, 45 bp, Artificial Sequence
  rrnB intrinsic terminator
aaacgaaaggctcagtcggaagactgggcctttcgttttatctgt

SEQ ID NO: 44 - DNA, 41 bp, Artificial Sequence
  T7t intrinsic terminator
aaccccttggggcctctaaacgggtcttgaggggttttttt

SEQ ID NO: 45 - DNA, 202 bp, Artificial Sequence
  T7t-tR2-rrnB triple terminator insert
  terminator 14..55: /note=“T7 terminator”
  terminator 59..102: /note=“tR2 terminator”
  terminator 132..176: /note=“rrnB triple terminator insert”
tctagatcggtggaaaccccttggggcctctaaacgggtcttgaggggtttttttaccgttaataacaggcctgctggtaatcgcaggcctttttattttgttacacgcacagcggctagcacgacgacaaacgaaaggctcagtcggaagactgggcctttcgttttatctgttaattttgtttaacttttactttatgta

SEQ ID NO: 46 - DNA, 21 bp, Artificial Sequence
  fixFW (primer)
tataggggaattcggagcggg

SEQ ID NO: 47 - DNA, 22 bp, Artificial Sequence
  fixRV_multi (primer)
tagcccgggatcctcggggctg

SEQ ID NO: 48 - DNA, 22 bp, Artificial Sequence
  lacZ_qPCR_FW
agcgcgatcccgtcgttttaca

SEQ ID NO: 49 - DNA, 20 bp, Artificial Sequence
  lacZ_qPCR_RV
caggctgcgcaactgttggg

SEQ ID NO: 50 - DNA, 20 bp, Artificial Sequence
  gapA_FW
gcaccaccaactgcctggct

SEQ ID NO: 51 - DNA, 20 bp, Artificial Sequence
  gapA_RV
cgccgcgccagtctttgtga

SEQ ID NO: 52 - DNA, 21 bp, Artificial Sequence
  nadA_before15_FW
acaggctctgtttggcggcac

SEQ ID NO: 53 - DNA, 19 bp, Artificial Sequence
  nadA_before15_RV
cgccagcgtttccacgggt

SEQ ID NO: 54 - DNA, 20 bp, Artificial Sequence
  nadA_after15_FW
agcgaacagcgtgcagcgta

SEQ ID NO: 55 - DNA, 20 bp, Artificial Sequence
  nadA_after15_RV
tgcgcagtgtaagagggggc

SEQ ID NO: 56 - DNA, 28 bp, Artificial Sequence
  P1-before RAP
ttaaccagtaacaacagaattctagccc

SEQ ID NO: 57 - DNA, 29 bp, Artificial Sequence
  P2-after RAP
ttattatctagaggatccccgggtgcatt

SEQ ID NO: 58 - DNA, 88 bp, Artificial Sequence
  T7A1
  promoter 1..67: /note=“T7 promoter”
tccagatcccgaaaatttatcaaaaagagtattgacttaaagtctaacctataggatacttacagccatcgagagggacacggcgaat

SEQ ID NO: 59 - DNA, 19 bp, Artificial Sequence
  GFP_FW
tggagagggtgaaggtgat

SEQ ID NO: 60 - DNA, 24 bp, Artificial Sequence
  GFP_RV
agcattgaacaccataagtcaaag

SEQ ID NO: 61 - RNA, 25 nt, Artificial Sequence
  5′-phosphorylated RNA adapter (for 3′RACE experiments)
aauggacucguaucacacccgacaa

SEQ ID NO: 62 - DNA, 22 bp, Artificial Sequence
  Forward primer 3′RACE
ttgggctagaattctgttgtta

SEQ ID NO: 63 - DNA, 25 bp, Artificial Sequence
  Reverse primer 3′RACE
ttgtcgggtgtgatacgagtccatt

SEQ ID NO: 64 - DNA, 114 bp, Artificial Sequence
  108nt insert to introduce additional, unique XhoI and SacI restriction enzyme recognition sites into plasmid pWM1015 (resulting in pWM3110)
gaattcatcgcgtgtgcgtattcgatcgaattggatcagctcgcgtccaggtagcgaaagccatttattgatggaccactcgagatcactagccgagctctaattgctaaattc

SEQ ID NO: 65 - DNA, 2695 bp, Artificial Sequence
  pWM3110
agaataaccctaaaaataaaccctgattttgcttatttagtcaatcaactaactactaattacagctttgagcttgaagaatttatagcccttagcggtaaatatgctaaaacactttaccgccttttaaagcaatttagaaccactggcaaagcttattttgagtgggacgagttttgtaaagttatggatataccacaagattatagacaaagcgaagtcgataaatggattttaaacctgctatcaaagaactttctaaagaacagcaatctttttgaccaaattagagtgccttttaaaaatcttgcttatgaaaaagaaaaaaccgcaggcgtgggcgtggcggcaaagtttcaggcattagctttacttttaaacctgaaaatatcgaaatgcaaaagctagaaaatgaaagtcaaaaaataatgagcgatgagcaaaaatatttaaagattttaaacaatatgaaacttaatcaagctagatttaattataatgacaagctttggcagtttaacgattttgattttaatgaatttaaaattattgcagtagagcttgtaagagatgaatacgagaatttaaactttggaaatcatatgcactttaatgctaaaaatcaagagcagttttttaaatgattgatactttaggaatgggattaggtaaaatttgctataattatatctttgaaggatacaagctatccgccacttgtgccaagtgtcgagcttgtaaggggtgcaaccccttaaccccactaataaaaatcaactaaatcagtgaatgatttttgaattttattaaaacaccaaaaaaggaaaatttaaaaatgcaaaaacatatcccgaagtgcatagcttagaagaaagccttgcgatacttaaaaaatataaagatgatttgactaaagagcaatacgaagccataagatcaaatataggtaattttgcaatagaagatatgtttttgaatgaaaaagatattattgataatgttaaaattataaaaggcgaagcaacagctaatgaatgtattgttgcattaaagaaagaatggggagtatgaatgcaaaatgtcaaaagttgccaccgctcattgcaatcctaaaaaacaacctgcattaaaccacaatgatagaaccaacgataatgctaagacaatcactaaagaacttacacatttaaatgaatactcttgcactagcgatgaagtgcgtaagaacatagaaaggctttataaaaaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataagcgtaccggttccaattttttcgcagtttaacttttccgacgcatttatcgtcttggtagtaaagatatattatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgatgataagctgtcaaacatga

SEQ ID NO: 66 - DNA, 32 bp, Artificial Sequence
  promoter of pWM3110 (constitutive promoter)
tttaagtcttagtttagtttttttggtataat

SEQ ID NO: 67 - DNA, 30 bp, Artificial Sequence
  arabinose-inducible promoter
gtttctccatacccgtttttttgggctagc

SEQ ID NO: 68 - DNA, 114 bp, Artificial Sequence
  Insert for integration of rut into pWM3100 (rut sequence in bold)
gagctctgccgtaccgtctatcaaactcaacgaccccttccttctccccatcgctacctcatatccgcacctcctcaaacgctacctcgaccagcctccctccctcccgtgctc

SEQ ID NO: 69 - DNA, 183 bp, Artificial Sequence
  Insert for integration of rrnB intrinsic terminator into pWM3100 (rrnB sequence in bold)
gagctctgccgtaccgtctatcaaactcaacgacaccatagcgtcctggatcattaacgtgatacgcagataccgttgcaccactctgttcacagcggctagcacgtcgacaaacgaaaggctcagtcggaagactgggcctttcgttttatctgttaattttgtttaactttaggagtgctc

SEQ ID NO: 70 - DNA, 179 bp, Artificial Sequence
  Insert for integration of T7t intrinsic terminator into pWM3100 (T7t sequence in bold)
gagctctgccgtaccgtctatcaaactcaacgacaccatagcgtcctggatcattaacgtgatacgcagataccgttgcaccactctgttcacagcggctagcacgtcgacaaccccttggggcctctaaacgggtcttgaggggttttttttaattttgtttaactttaggagtgctc

SEQ ID NO: 71 - DNA, 214 bp, Artificial Sequence
  Insert for integration of the three intrinsic terminators T7t, tR2 and rrnB into pWM 3100
gagctctctagatcggtggaaaccccttggggcctctaaacgggtcttgaggggtttttttaccgttaataacaggcctgctggtaatcgcaggcctttttattttgttacacgcacagcggctagcacgacgacaaacgaaaggctcagtcggaagactgggcctttcgttttatctgttaattttgtttaacttttactttatgtagtgctc

SEQ ID NO: 72 - DNA, 29 bp, Artificial Sequence
  RAP ID# 5713_mut_loop2
gacaacatgagggcggttcagcagcacta

SEQ ID NO: 73 - DNA, 29 bp, Artificial Sequence
  RAP ID# 5713_mut_2gg
gacggcatgagggcagttcagcagcacta

SEQ ID NO: 74 - DNA, 29 bp, Artificial Sequence
  RAP ID# 5713_mut_3gg
gacggcatgagggcagggcagcagcacta

SEQ ID NO: 75 - DNA, 23 bp, Artificial Sequence
  RAP ID# 5713_mut_0-6
gacaacatgagaacagttcagca

SEQ ID NO: 76 - DNA, 29 bp, Artificial Sequence
  RAP ID# 5713_mut_stem3
gacaacaacagaacagttgtgcagcacta

SEQ ID NO: 77 - DNA, 29 bp, Artificial Sequence
  RAP ID# 5713_mut_noStruct
gacaacacccgaacagtcccgcagcacta

SEQ ID NO: 78 - DNA, 70 bp, Artificial Sequence
  +70 RBS
taataactatacgtatggtaatcgcaggccttattccgagctcaataattgttaactttaagaaggagga

SEQ ID NO: 79 - DNA, 3150 bp, Artificial Sequence
  lacZ gene in pBAD13
atgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataa

SEQ ID NO: 80 - DNA, 99 bp, Artificial Sequence
  cobC-derived +99 nt insert
tgcattctcgaccccgatggcacggctattgaggacgcgtagcgtcgcgaatttttggttgatatcaatggcgctccaacacccctggtcaacgcgaaa

SEQ ID NO: 81 - DNA, 49 bp, Artificial Sequence
  +49 nt insert
tttgttactggttaacctgaaacgccagtctgcccatacgccactgcgt

SEQ ID NO: 82 - DNA, 30 bp, Artificial Sequence
  Forward primer pET21_Insert_FW_NheI
atgctagcacccgtttttttgggctagaat

SEQ ID NO: 83 - DNA, 32 bp, Artificial Sequence
  Reverse primer pET21_Insert_RV_HindIII
ataagcttttattattatttttgacaccagac

SEQ ID NO: 84 - DNA, 18 bp, Artificial Sequence
  inhibiting RAP consensus sequence
wydamghmshkrmwsdwm

SEQ ID NO: 85 - DNA, 12 bp, Artificial Sequence
  inhibiting RAP consensus sequence
  misc_feature 3: /note=“wherein ”N“ may be A or T or C or G”
  misc_feature 10: /note=“wherein ”N“ may be A or T or C or G”
gmnkatyvsntk

SEQ ID NO: 86 - DNA, 10 bp, Artificial Sequence
  inhibiting RAP consensus sequence
gccakgakcg

SEQ ID NO: 87 - DNA, 11 bp, Artificial Sequence
  inhibiting RAP consensus sequence
bhmvdsvrsrw

SEQ ID NO: 88 - DNA, 47 bp, Artificial Sequence
  RAP ID# 4599
cgggcatattgcgtgatttgtgccaaccgatgattactttcccctgc

SEQ ID NO: 89 - DNA, 61 bp, Artificial Sequence
  RAP ID# 1436
cagcgccatgagcgtgatccacataccaataaattcgtcccagacaatgctgccatgatcg

SEQ ID NO: 90 - DNA, 35 bp, Artificial Sequence
  RAP ID# 6819
ccagtcggcaaacattcccatccagacaccaacca

SEQ ID NO: 91 - DNA, 49 bp, Artificial Sequence
  RAP ID# 1760
accatcaaccactgaccgacgattagcttacgcagttgcgctcgccctg

SEQ ID NO: 92 - DNA, 39 bp, Artificial Sequence
  RAP ID# 1086
gcgtttcaaatgcgcatcaacctgccacactcccccaca

SEQ ID NO: 93 - DNA, 63 bp, Artificial Sequence
  RAP ID# yh1
tcatagacaagacaatcctaagcaaaaagaacaaagctgtaggcatcatactacctgactatc

SEQ ID NO: 94 - DNA, 37 bp, Artificial Sequence
  RAP ID# yh2
gagcaaatcaggcagtccttacccactcccctccctg

SEQ ID NO: 95 - DNA, 76 bp, Artificial Sequence
  RAP ID# yh3
aacttcaaaaaaaattacaaaaagtgctttctgaactgaacaaaaaagagtaaagttagtcgcgtaaagtacatct

SEQ ID NO: 96 - DNA, 40 bp, Artificial Sequence
  RAP ID# yh4
ttcccaacgtgttagaattataggcatgacccactgtgcc

SEQ ID NO: 97 - DNA, 87 bp, Artificial Sequence
  RAP ID# yh5
gtacgagcggaccccacagttttaagcctaaacttggccttatgtagaatttcttgatatcattatcatctttgtcgtagtcccatc

SEQ ID NO: 98 - DNA, 54 bp, Artificial Sequence
  RAP ID# yh6
gcaaaatgagtacttagacatgctctgtatttgtgctgtacagtacggtaacac

SEQ ID NO: 99 - DNA, 22 bp, Artificial Sequence
  RAP ID# yh7
gaggcacacactcacacacccc

SEQ ID NO: 100 - DNA, 58 bp, Artificial Sequence
  RAP ID# yh8
gggaattcggagcgggatcggttgcattagctaagcctgagaaggtgatgttgttgcc

SEQ ID NO: 101 - DNA, 28 bp, Artificial Sequence
  RAP ID# yh9
atatcagccatgtacatccctgtccccc

SEQ ID NO: 102 - DNA, 60 bp, Artificial Sequence
  RAP ID# yh10
gcccacaagattccacgatagatatcaaggatttaaagctgattcagtgtatagtacatc

SEQ ID NO: 103 - DNA, 48 bp, Artificial Sequence
  RAP ID# yh11
ccataccagcatgatggataccaaaaccaaattgaagaatctccttcc

SEQ ID NO: 104 - DNA, 31 bp, Artificial Sequence
  RAP ID# yh12
attgcttttccacacggattccaaccactgc

SEQ ID NO: 105 - DNA, 36 bp, Artificial Sequence
  RAP ID# yh13
attgcttttccacacggattccaaccgatatcatac

SEQ ID NO: 106 - DNA, 32 bp, Artificial Sequence
  RAP ID# 14
aagcactacccacctgacaatgtcttcatccc

SEQ ID NO: 107 - DNA, 39 bp, Artificial Sequence
  RAP ID# yh15
agcatacaaaatatacctttctcaaacaagaaacatccc

SEQ ID NO: 108 - DNA, 35 bp, Artificial Sequence
  RAP ID# yh16
gagccttaaacactgacttcccattccctctcccc

SEQ ID NO: 109 - DNA, 29 bp, Artificial Sequence
  RAP ID# yh17
gcagcccacacatcatacacacacacccc

SEQ ID NO: 110 - DNA, 72 bp, Artificial Sequence
  RAP ID# yh18
gcagcttaaactgcctgaccggtaagaaactgaccaacatgcgtgcttccggtactgacgaagccttcatcc

SEQ ID NO: 111 - DNA, 20 bp, Artificial Sequence
  RAP ID# yh19
gcagcccacaaaccacaccc

SEQ ID NO: 112 - DNA, 40 bp, Artificial Sequence
  RAP ID# yh20
gcagcatgaaagtcacagttctaaacattcattctgtgcc

SEQ ID NO: 113 - DNA, 39 bp, Artificial Sequence
  RAP ID# yh21
gcagcagacacacgtacacacacacatacataacctatc

SEQ ID NO: 114 - DNA, 33 bp, Artificial Sequence
  RAP ID# yh22
gcagcatatcagccatgtacatccctgtccccc

SEQ ID NO: 115 - DNA, 26 bp, Artificial Sequence
  RAP ID# yh23
gcagcaacacacacacacacacaccc

SEQ ID NO: 116 - DNA, 69 bp, Artificial Sequence
  RAP ID# yh24
gcagctttacttaacagttgagctaatttacactcccatggatagtgtataagtgtttcattttcgccc

SEQ ID NO: 117 - DNA, 36 bp, Artificial Sequence
  RAP ID# yh25
gcagcatgaaccaaacaccttccactagtcccatcc

SEQ ID NO: 118 - DNA, 45 bp, Artificial Sequence
  RAP ID# yh26
gcagcaacacacagcacacacatacacccaccgtactaagcatcc

SEQ ID NO: 119 - DNA, 57 bp, Artificial Sequence
  RAP ID# yh27
gagccaactacatcgaccaatttaccagaggattcaccattatgatcaccatcttcc

SEQ ID NO: 120 - DNA, 54 bp, Artificial Sequence
  RAP ID# yh28
gcagcagcactcatcggttcatcatctatgtaaaggtgcttctccaccatgtgc

SEQ ID NO: 121 - DNA, 40 bp, Artificial Sequence
  RAP ID# yh29
gcagcaaccacacatctatacggccttgaaaaccccatcc

SEQ ID NO: 122 - DNA, 53 bp, Artificial Sequence
  RAP ID# yh30
aagcactaaaggcccattcttgctctatgtatctgtgacttaagatctgccac

SEQ ID NO: 123 - DNA, 24 bp, Artificial Sequence
  RAP ID# yh31
acggccaccagactcattcctgcc

SEQ ID NO: 124 - DNA, 46 bp, Artificial Sequence
  RAP ID# yh32
attgcttttccacacggattccaaccgctatccaaacactcatccc

SEQ ID NO: 125 - DNA, 41 bp, Artificial Sequence
  RAP ID# yh33
gcagcactcacttacacattcacacacacaacatacacccc

SEQ ID NO: 126 - DNA, 43 bp, Artificial Sequence
  RAP ID# yh34
gcagcgttagtacatacccttcctcaccattgttcccccatcc

SEQ ID NO: 127 - DNA, 65 bp, Artificial Sequence
  RAP ID# yh35
gagctaagacacagatacccagaaagatccaaacctctaccgcaaagtcaaggtagacatttccc

SEQ ID NO: 128 - DNA, 26 bp, Artificial Sequence
  RAP ID# yh36
gagtcccacacacacaccacaccacc

SEQ ID NO: 129 - DNA, 29 bp, Artificial Sequence
  RAP ID# yh37
cactttgtactttattggagttttcctcc

SEQ ID NO: 130 - DNA, 41 bp, Artificial Sequence
  RAP ID# yh38
gagcctacaaattaaatctagccagaagcattctatcttcc

SEQ ID NO: 131 - DNA, 81 bp, Artificial Sequence
  RAP ID# yh39
gcagcaacttcaaaaaaaattacaaaaagtgctttctgaactgaacaaaaaagagtaaagttagtcgcgtaaagtacatct

SEQ ID NO: 132 - DNA, 76 bp, Artificial Sequence
  RAP ID# yh40
cagatcaggtcagctcaggataattcaaattcagataatgaggcacagactaatcataacacgaaaattgtcatcc

SEQ ID NO: 133 - DNA, 68 bp, Artificial Sequence
  RAP ID# yh41
tcacataacattcacacatgcacacatgtaccacagccatgcccacataccacagccacgcatcatcc

SEQ ID NO: 134 - DNA, 45 bp, Artificial Sequence
  RAP ID# yh42
gtagtaatcgtcttcattctcatggtgttcatcgttctctatgcc

SEQ ID NO: 135 - DNA, 82 bp, Artificial Sequence
  RAP ID# yh43
gcagcacttatggtccatattagtgtctactaccctggttatggaaggttatgataccgcactactgaacgcactgtatgcc

SEQ ID NO: 136 - DNA, 78 bp, Artificial Sequence
  RAP ID# yh44
gagcatattggacaggttgtacttgattagaaaacacaatcatgcggtaatattaagccatactcaacagcgccatcc

SEQ ID NO: 137 - DNA, 52 bp, Artificial Sequence
  RAP ID# yh45
gagcatacgtagttgtagttttcattttgatggcttaaccttctttgtcagc

SEQ ID NO: 138 - DNA, 17 bp, Artificial Sequence
  eukaryotic transcription enhancing RAP consesus sequence
  misc_feature 6: /note=“wherein ”N“ may be A or T or C or G”
  misc_feature 15: /note=“wherein ”N“ may be A or T or C or G”
mmrcanmwbaywccnam

SEQ ID NO: 139 - DNA, 12 bp, Artificial Sequence
  enhancing RAP consensus
  misc_feature 3: /note=“wherein N may be 0 to 3 nucleotides idependently selected from the group consisting of A, T, C, and G”
  misc_feature 6: /note=“wherein ”N“ may be A or T or C or G”
  misc_feature 9: /note=“wherein ”N“ may be A or T or C or G”
canmmnswnmmh

(19) United States
(12) Patent Application Publication
(10) Pub. No.: US 2018/0073026 A1
(43) Pub. Date: Mar. 15, 2018

(54) NOVEL EXPRESSION REGULATING RNA-MOLECULES AND USES THEREOF

(71) Applicant: Universität Wien, Wien (AT)

(72) Inventors: Renée Schroeder, Wien (AT); Nadezda Sedlyarova, Wien (AT)

(73) Assignee: Universität Wien, Wien (AT)

(21) Appl. No.: 15/563,287

(22) PCT Filed: Mar. 29, 2016
(86) PCT No.: PCT/EP2016/056827

§ 371 Date: Sep. 29, 2017

(30) Foreign Application Priority Data
Apr. 1, 2015 (EP) 15162198.4

(51) Int. Cl. C12N 15/115; C12N 15/67

(52) U.S. Cl. CPC C12N 15/115; C12N 15/67; C12N 2310/11; C12N 2310/16; C12N 2320/50; C12N 2320/11

(57) ABSTRACT

The present application relates to a RNA molecule comprising a RNA-polymerase binding aptamer, wherein said RNA-polymerase binding aptamer has a length of 15 to 60 nt, wherein said RNA-Polymerase binding aptamer binds to a RNA-polymerase with a K_{D} of 50 nM or lower. Furthermore, the application discloses a DNA molecule comprising a sequence encoding an RNA-polymerase binding aptamer of the invention, and a method of producing a protein or RNA of interest comprising providing a vector according to the invention, said vector comprising an expression, introducing said vector into a host cell, and culturing said host cell in culture medium under conditions inducing transcription from the promoter of the expression vector, and optionally recovering the protein of interest from the host cell or culture medium. Furthermore, the application pertains methods for in vitro transcription and expression employing the RNA-polymerase binding apatamers.

### TECHNICAL FIELD OF THE INVENTION

The present invention relates to the field of biology, particularly to the field of biotechnology. More particular, the present invention relates to the field of RNA-Biology and its implication in regulating gene expression, e.g. for expressing genes of interest in a host cell.

### BACKGROUND OF THE INVENTION

In bacteria all steps of transcription, from initiation to elongation and termination, are prone to regulation by trans-acting proteins and small RNAs. Additionally, the nascent RNA itself contains many cis-acting regulatory signals. Riboswitches are cis-acting RNA elements mainly encoded in the 5′ UTR of the nascent RNA that directly sense small metabolites, ions, temperature, or pH, leading to regulation of transcription, translation, or RNA processing (A. Serganov, E. Nudler, Cell. 152, 17-24 (2013); and S. Proshkin, A. Mironov, E. Nudler, Biochim. Biophys. Acta (2014), doi:10.1016/j.bbagrm.2014.04.002). Termination signals are another example of regulatory RNA signals that can be categorized into two major groups: intrinsic and factor-dependent terminators (E. Nudler, M. E. Gottesman, Genes to Cells. 7, 755-768 (2002); and J. M. Peters, A. D. Vangeloff, R. Landick, J. Mol. Biol. 412, 793-813 (2011)). GC-rich RNA hairpins followed by a stretch of uridines are intrinsic signals that cause termination independently of cofactors. Termination at factor-dependent signals requires the presence of protein factors, such as Rho, recognizing particular RNA regions. Nascent RNA can also increase the processivity of RNAP providing factor-dependent or -independent antitermination signals. For example, phage HK022 put RNA binds directly to RNAP throughout elongation rendering it resistant to termination (R. Sen, R. A. King, R. A. Weisberg, Mol. Cell. 7, 993-1001 (2001); and N. Komissarova et al., Mol. Cell. 31, 683-94 (2008)). Factor-dependent cis-acting nut RNA (box A+box B) assists phage λ N protein during processive antitermination together with bacterial NusA, NusB, S10. No common attributes could until today be found. All of the known regulatory elements, however, request a distinct secondary structure which makes them difficult to design. Hence, there is a need for regulatory sequences of RNA not needing distinct secondary structures.

Heterogenous expression of sequences in host cells is often prone to sequences having regulatory effects when introduced into a host cell for expression. They may inhibit expression, e.g. in terms of termination of transcription or translation. For example, a heterogenous sequence may contain sequences having inhibitory effects on expression in the host cell. This would result in an unwanted restriction on expression. Furthermore, output of the target sequence expression in host cells can be affected by surrounding sequences with regulatory effects in the host cell. Moreover, unwanted transcription of the reverse complement strand, e.g. by unidentified promoter sequences, may cause a loss transcription efficiency due to mutual hindrance of the polymerases transcribing the opposing strands. There is hence a need for a tool to allow proper expression of sequence of interest in a host cell irrespective of the potential presence of inhibitory elements within the sequence to be expressed. In particular tools are missing or desired that allow an efficient production of recombinant proteins in host cells.

While start and amount of transcription may often be regulated or enhanced using promoters, such as inducible promoters, the regulation of translation efficiency is yet insufficiently solved.

Furthermore, the expression of a certain sequence, e.g. leading to a protein, in a heterologous host cell may cause difficulties when the protein has deleterious effects on the host cell. Nowadays, expression systems are employed which make use of inducible promoters. Such promoters effectively initiate transcription only in case a certain stimulus is present, e.g. a certain compound is added to the culturing medium. However, such promoters often promote basal expression in the un-stimulated environment leading to a certain leakiness of the promoter. In case of proteins with deleterious effects this may have adverse effects and may lead to ineffective expression once the expression shall be induced.

The inventors now provide a novel class of regulating RNA aptamers which provide a powerful in overcoming the above outlined drawbacks.

### DESCRIPTION OF THE INVENTION

The inventors interestingly found short RNA-polymerase binding aptamers (termed RAPs) that interact with RNA-polymerases with high affinity. The inventors did identify several naturally occurring RAPs. They furthermore, showed that these aptamers are able to regulate expression of the DNA-sequences they are encoded on. Thereby a completely new tool is provided to regulate RNA-polymerases and thereby a tool for regulating expression of DNA-encoded molecules, like RNAs and/or Proteins, in a host cell.

Hence, the present invention in one aspect relates to a RNA molecule comprising a RNA-polymerase binding aptamer, wherein said RNA-polymerase binding aptamer has a length of 15 to 60 nt, and wherein said RNA-polymerase binding aptamer binds RNA-polymerase with a high affinity, preferably with a K_{D} of 50 nM or lower, preferably with a K_{D} of 10 nM or lower.

The invention also relates to a DNA-molecule encoding a RNA-polymerase binding aptamer according to the present invention. The invention further relates to an expression cassette comprising a sequence encoding the RNA-polymerase binding aptamer according to the present invention and a promoter, wherein the sequence encoding said aptamer is operatively linked to said promoter.

The invention furthermore relates to a vector, preferably a vector for expression of a sequence of interest, said vector comprising a sequence encoding a RNA-polymerase binding aptamer according to the present invention. The vector in a preferred embodiment further comprises a promoter, a multiple cloning site for integration of the sequence of interest. The vector may comprise a DNA-molecule according to the invention, preferably an expression cassette according to the invention. Furthermore, in one aspect of the invention the expression cassette comprises the reverse complement sequence of the sequence encoding an inhibitory RNA-polymerase binding aptamer downstream of the promoter. In one aspect of the invention, the expression cassette comprises a promoter, a DNA sequence to be expressed or a multiple cloning site for inserting a DNA sequence to be expressed and a DNA sequence coding for a 3′UTR downstream of said DNA sequence to be expressed or said multiple cloning site, wherein the DNA sequence coding for a 3′UTR comprises a the reverse complement of a DNA sequence encoding for an inhibiting RNA-Polymerase binding aptamer according to the invention.

Also encompassed by the present invention is a host cell comprising a RNA-polymerase binding aptamer according to the present invention. Furthermore, the invention relates to a host cell comprising DNA-molecule comprising a sequence encoding a RNA-polymerase binding aptamer according to the present invention.

Furthermore, the invention relates to a method for expression of a sequence of interest, preferably a DNA sequence, comprising the steps of:
providing a vector according to the present invention, said vector comprising an expression cassette according to the present invention
introducing said vector into a host cell, and
culturing said host cell in culture medium under conditions inducing or allowing transcription from the promoter of the expression vector.

The invention, furthermore, relates to the use of RNA-molecule or the DNA-molecule or the expression cassette or the vector of the present invention to regulate expression of a sequence to be expressed. In other words, the RNA-polymerase binding aptamer according to the invention is employed in methods for expressing a sequence to be expressed, e.g. in cis by encoding the aptamer on the same DNA molecule as the sequence to be expressed is encoded on.

### FIGURE LEGEND

FIG. 1. Distribution of RNA-polymerase binding aptamers (RAP) within the E. coli genome. (A) RAPs categorization according to their location on the E. coli genome. Annotated genes are shown as grey arrow. RAP position is depicted as triangles. (B) Distribution of RAPs from different categories within the E. coli genome. (C)-(D) Analysis of sense (C) and antisense (D) RAP location within annotated genes. The brighter line shows the counts of RAPs found in the respective region of all annotated ORFs (+10% up/downstream). The background (in black) shows the respective signals from 1000 randomized runs.

FIG. 2. Effect of activating RAPs in the GFP reporter system (driven by bacterial RNA-polymerase (RNAP)). (A) A schematic overview of the reporter construct used to test the effect of activating RNA-polymerase binding aptamers (RAP; rectangle) placed upstream of the GFP reporter gene (GFP). “P” (grey triangle) indicates the location of a constitutive bacterial RNAP promoter. TSS: transcription start site; RBS: ribosomal binding site. The location of qRT-PCR amplicon is depicted below (lenticular bar). (B) E. coli cells transformed with GFP reporter plasmids containing different RAPs were grown on LB agar plates followed by fluorescence intensity measurements (left panel—GFP mode). The same plate was also captured under visible light showing equal bacterial density of strains with RAP-GFP constructs (right panel—light mode). (C) Levels of reporter gene transcripts containing different RAPs (indicated with their RAP ID) when transcribed by bacterial RNAP. qRT-PCR data, normalized to the house-keeping gapA levels. RAP IDs #5713 and #14908 significantly up-regulate transcription in comparison to constructs comprising no RAP. Control constructs #7523 and #3468 did not change transcript levels in comparison to no RAP construct. Values represent means±SD, n≧3; **p<0.01; *p<0.05; ns: not significant (tested with T-test, equal variance).

FIG. 3. Effect of activating RAPs in the GFP reporter system (driven by phage T7 RNAP). (A) A schematic overview of the reporter construct used to test the effect of activating RNA-polymerase binding aptamers (RAP; rectangle) placed upstream of the GFP reporter gene. “T7′P” (triangle) indicates the location of phage promoter recognized by phage T7 RNAP. TSS: transcription start site; RBS: ribosomal binding site. The location of qRT-PCR amplicon is depicted below (lenticular bar). (B) E. coli cells transformed with GFP reporter plasmids containing different RAPs were grown on LB agar plates followed by fluorescence intensity measurements (GFP mode). (C) Levels of reporter gene transcripts containing different RAPs upstream (indicated with their numbers) when transcribed by phage T7 RNAP. qRT-PCR data, normalized to the house-keeping gapA levels. RAP IDs #5713 and #14908 did not change transcript levels in comparison to no RAP construct. Values represent means±SD, n≧3; ns: not significant (tested with T-test, equal variance).

FIG. 4. Checking RAPs promoter activity. (A) A schematic overview of the reporter construct used to test RAP promoter activity. The original constitutive bacterial promoter (triangle) was replaced by RAP (rectangle) upstream of the GFP reporter gene. TSS: transcription start site; RBS: ribosomal binding site. The location of qRT-PCR amplicon is depicted below (lenticular bar). (B) Levels of reporter gene transcripts containing different RAPs instead of constitutive bacterial RNAP promoter. qRT-PCR data, normalized to the house-keeping gapA levels. Values represent means±SD, n≧2.

FIG. 5. Checking RAP ID #5713 (SEQ ID NO:2) activity upstream of promoter. (A) A schematic overview of the reporter constructs used in the assay. Tested RAP ID #5713 or control reverse complement sequence (RAP-marked rectangle) was placed upstream the original constitutive bacterial promoter (grey triangle). TSS: transcription start site; RBS: ribosomal binding site. The location of qRT-PCR amplicon is depicted below (lenticular bar). (B) Levels of reporter gene transcripts in the assay. qRT-PCR data, normalized to the house-keeping gapA levels. Values represent means±SD, n≧2; **P<0.01; ns, not significant (tested with T-test, equal variance).

FIG. 6. RAP ID #5713 promotes read-through intrinsic terminators. (A) A schematic overview of the reporter construct used to test the antitermination effect of RAPs (red rectangle), placed upstream intrinsic terminator “Term” (schematically shown as hairpin). “P” (grey triangle) indicates the location of constitutive bacterial RNAP promoter. TSS: transcription start site; RBS: ribosomal binding site. The location of qRT-PCR amplicon is depicted below (lenticular bar). (B) Levels of reporter transcripts without RAP sequence containing no intrinsic terminator (No RAP_No term) or with rrnB intrinsic terminator (No RAP_rrnB)/with T7t intrinsic terminator (No RAP_T7t). qRT-PCR data, normalized to the house-keeping gapA levels. Values represent means±SD, n=3. (C) E. coli cells transformed with described reporter plasmids containing heterologous intrinsic terminators (marked on top) were grown on LB agar plates followed by fluorescence intensity measurements (left panel—GFP mode). The same plate was also captured under visible light showing equal bacterial density of strains (right panel—light mode). (D) Secondary structure predictions for RAP ID #5713—intrinsic terminators constructs (with rmB—upper panel, with T7t—lower panel), obtained by RNA fold software (Vienna RNA Package). Sequence of RAP ID #5713 is 111 nts away from the intrinsic terminators and does not affect its folding. (E) Levels of reporter gene transcripts with rrnB terminator in the absence and in the presence of RAP ID #5713. qRT-PCR data, normalized to the house-keeping gapA levels. RAP ID #5713 activate read-through the rrnB terminator (=RAP act as antiterminator) up to 8 times in comparison to no RAP construct. Values represent means±SD, n=3. (F) Levels of reporter gene transcripts with T7t terminator in the absence and in the presence of RAP ID #5713. qRT-PCR data, normalized to the house-keeping gapA levels. RAP ID #5713 activate read-through the T7t terminator (=RAP act as antiterminator) up to 14 times in comparison to no RAP construct. Values represent means±SD, n=3.

FIG. 7. RAP ID #5713 (SEQ ID NO:2) promotes antitermination for Rho-dependent terminators. (A) A schematic of the reporter construct used to test the antitermination effect of RAPs (rectangle), placed upstream the Rho-dependent termination site rut (rut-marked rectangle). “P” (grey triangle) indicates the position of the constitutive bacterial RNAP promoter. TSS: transcription start site; RBS: ribosomal binding site. The location of qRT-PCR amplicon is depicted below (lenticular bar). (B) E. coli cells transformed with the rut-containing reporter plasmids were grown on LB agar plates in the absence of BCM followed by fluorescence intensity measurements (left panel—GFP mode). The same plate was also captured under visible light showing equal bacterial density of strains (right panel—light mode). (C) E. coli cells transformed with the rut-containing reporter plasmids were grown on LB agar plates in the presence of BCM (added up to 8 μg/ml, final concentration) followed by fluorescence intensity measurements (left panel—GFP mode). The same plate was also captured under visible light showing equal bacterial density of strains (right panel—light mode).

FIG. 8. Challenging antiterminatory activity of RAP ID #5713 (SEQ ID NO:2). (A) A schematic overview of the reporter construct used to test antitermination effect of RAPs (rectangle), placed upstream the stretch of 3 heterologous terminators T7t-tR2-rrnB (schematically shown as hairpins, for sequence see SEQ ID NO:45). “P” (grey triangle) indicates the location of constitutive bacterial RNAP promoter. TSS: transcription start site; RBS: ribosomal binding site. (B) E. coli cells transformed with described three terminator reporter plasmids were grown on LB agar plates followed by fluorescence intensity measurements (left panel—GFP mode). The same plate was also captured under visible light showing equal bacterial density of strains (right panel—light mode). (C) A schematic overview of the reporter construct used to test antitermination effect of several RAP ID #5713 (SEQ ID NO:2) cloned in tandem (rectangle, n=1, 2 or 3), placed upstream the rrnB terminator (schematically shown as hairpin). “P” (grey triangle) indicates the location of constitutive bacterial RNA-polymerase (RNAP) promoter. TSS: transcription start site; RBS: ribosomal binding site. (D) E. coli cells transformed with described 1×, 2× or 3×RAP ID #5713 reporter plasmids were grown on LB agar plates followed by fluorescence intensity measurements (left panel—GFP mode). The same plate was also captured under visible light showing equal bacterial density of strains (right panel—light mode).

FIG. 9. Checking antitermination effect of RAP ID #5713 in in vitro transcription system. (A) A schematic of the template used for testing RAPs antitermination activity in vitro. “P” (grey triangle) indicates the location of constitutive strong bacterial RNAP promoter T7A1; TSS: transcription start site. RAP (rectangle) is located upstream of intrinsic terminator rrnB (shown schematically as hairpin). (B) Representative single round run-off transcription assays (6% TBE-UREA gel). The efficiency of intrinsic transcription termination on template with RAP ID #5713 was compared to the control template with reverse complement sequence Rev #5713. The products of intrinsic termination are marked with arrows. The line on top (“run off”) indicates the run-off product. (C) Quantification of termination efficiency was performed using single round run-off transcription assays as described in (B). To calculate termination efficiency, the intensity of intrinsic termination was divided by the total intensity (intrinsic termination+run-off). Termination activity of RNAP on the template with RAP ID #5713 is at least twice reduced when compared to control Rev #5713 when tested in vitro. Values represent means±SD, n=3.

FIG. 10. In silico analysis of identified transcription enhancing RAPs. (A) In silico analysis of the secondary structure of RAP ID #5713 (SEQ ID NO:2). RNAfold prediction (Fold algorithms and basic options: “minimum free energy (MFE) and partition function”, implying calculating the partition function and base pairing probability matrix in addition to the minimum free energy (MFE) structure). (B) Sequence logo presentation of the sequence analysis (Schneider T D, Stephens R M (1990). “Sequence Logos: A New Way to Display Consensus Sequences”. Nucleic. Acids. Res. 18 (20): 6097-6100). Prediction was performed with The MEME Suite 4.10.0: Motif-based sequence analysis tools. Reported activating RAP sequences were submitted to MEME program with the following parameters: “site distribution”—one per sequence; “search given strand only”. Width of the sequence is 23 nt. The E-value is 8.8e-009.

FIG. 11. RAPs leading to transcription termination. (A) Schematic overview of the reporter construct used to test the effect of RAPs (rectangle). “P” (triangle) indicates the location of several promoters used for different experimental setups (inducible/constitutive bacterial RNAP promoter or phage T7 promoter). TSS: transcription start site; RBS: ribosomal binding site. The location of qRT-PCR amplicons and Northern Blot probes is depicted below (lenticular bars; for Northern Blots named P1 and P2, respectively). (B)+(C) Levels of reporter gene transcripts containing different RAPs upstream (indicated with their RAP IDs). qRT-PCR data, normalized to the house-keeping gapA levels: (B) transcription from RNAP promoter; (C) transcription from phage T7 promoter. (D) Northern blot analysis of transcripts derived from RAP-containing reporter constructs. Numbering on the top indicates the RAP cloned into the reporter construct. Upper panel “P2”: the result of hybridization with the probe located downstream of the RAP. Middle panel “P1”: the result of hybridization with the probe upstream of the RAP. The arrow indicates the stable transcript ˜150 nts long. Lower panel: loading control (SYBR-stained denaturing Urea PAGE).

FIG. 12. RAPs cause Rho-dependent transcription termination. (A) Effect of BCM (Rho inhibitor) on reporter gene expression containing diverse RAPs. qRT-PCR data, normalized to the house-keeping gapA levels before (dark grey bars) and after BCM treatment (light grey bars) for 20′ with final concentration 24 μg/ml. Numbering on the bottom indicates the RAP cloned into the reporter construct. (B) Schematic of the template used for in vitro transcription with tested RAPs (shown as a rectangle). (C)-(D) Representative run-off transcription assays. Pre-formed elongation complexes were chased with NTPs in the absence of Rho (lanes 1, 4, 7, 10, 13), in the presence of Rho (lanes 2, 5, 8, 11, 14) or with Rho and NusG (lanes 3, 6, 9, 12, 15). 15mut sequence was used as a negative control. Grey bars indicate the run-off product (top fraction) and the products of preliminary transcription termination (lower fraction).

FIG. 13. RAP ID #15 induces Rho-dependent termination within its host gene in response to stress. (A) Schematic overview of the nadD gene ORF with the location of the qRT-PCR primers used for measurements of endogenous nadD transcript levels upstream (lighter bar) and downstream (dark bar) the RAP ID #15 domain. (B) qRT-PCR data, nadD mRNA levels upstream and downstream of RAP ID #15 under various growth conditions. Upstream mRNA levels are set to 100% and downstream levels are normalized to the upstream levels.

FIG. 14. RAPs sequence analysis. (A) MEME-predicted motif for 10% of all identified RAPs. (B) Exemplary screen shot of deep sequencing results of genomic SELEX. Mapped reads form a peak, representing RAP ID #15 data for intragenic RAP ID #15, presented using two different scales (see track annotations). Upper: whole nadD gene view with RAP #15 (shown schematically with red arrow). Lower: zoomed-in fragment of nadD gene with RAP #15 tested sequence.

FIG. 15. Effect of inhibiting RAPs in the GFP reporter system. (A) A schematic overview of the reporter construct used to test the effect of RAPs (rectangle) placed upstream of the GFP reporter gene. “P” (grey triangle) indicates the location of a constitutive bacterial RNAP promoter. RBS is the ribosome binding site. (B)-(I) E. coli cells transformed with GFP reporter plasmids containing different RAPs (marked with RAP ID numbers) were grown on LB agar plates followed by fluorescence intensity measurements (GFP mode, upper panel). The same plate was captured under visible light showing the bacterial density of strains with RAP-GFP fusions (light mode, lower panel). (J) Mutations in the minimal sequence of RAP ID #15 (SEQ ID NO:25) that lead to the loss of down-regulating activity. Changed nucleotides (transitional mutations) are underlined and shown in grey (15 mut). (K) RAP ID #15 sequence (SEQ ID NO:25) translated into the corresponding protein sequence. RAP ID #15 is located within the essential nadD part, encoding conservative region of the Nicotinate-mononucleotide adenylyltransferase enzyme. Interestingly, the introduced mutations are within the sequences encoding 2 out of 6 conserved catalytic amino acids (underlined). (F) Beta-galactosidase assay results. Upper a schematic overview of the reporter construct used to test the effect of RAPs (rectangle) placed upstream of the reporter gene lacZ. Lower. β-galactosidase activity expressed by E. coli with different RAP-lacZ reporters. Numbering on the bottom indicates the insert (RAP or mutant) in the reporter construct.

FIG. 16. 3′RACE analysis for the RAP ID #15 (SEQ ID NO:25)-containing transcripts. (A) Schematic overview of 3′RACE procedure. The 5′-phosphorylated adapter was ligated to the 3′ end of total RNA isolated from E. coli transformed with the RAP-lacZ reporter construct. The obtained product was subjected to reverse transcription followed by PCR using primers indicated in colors. The PCR products were separated on gel, purified and cloned for subsequent sequencing. (B) Alignment of sequences obtained by 3′RACE: RAP-containing transcripts, representing the products of the preliminary RAP-dependent transcription termination. (C) 3′RACE analysis for the RAP ID #15 (SEQ ID NO:25)-containing transcripts before and after BCM treatment. 3′RACE PCR products resulting from two biological replicates were separated on 6.5% polyacrylamide gel. The prevailing short transcript with RAP ID #15 is labeled with ST. The non-specific amplification product is marked with an asterisk (*). The addition of BCM (Rho inhibitor) to the bacterial culture leads to the pronounced accumulation of longer RAP-containing transcripts (marked with a bar), at the same time decreasing the levels of the ST transcript (pointed with arrows).

FIG. 17. Inhibiting RAPs promote RNAP pausing in cis. (A) Upper panel: a schematic of a RAP-containing template used for the in vitro transcription assay. “P” (grey triangle) indicates the T7A1 promoter. Lower panel: representative single-round runoff assay (in solution) utilizing the template with RAP#15 (lanes 1-8) and the reverse complement control 15rev (lanes 9-16). Transcripts were analyzed 10, 20, 30, 40, 60, and >110 seconds after initiation of transcription. The RAP#15 domain and 15rev control sequence in the transcripts are indicated by dark-grey and light-grey rectangles, respectively. Transcript accumulation suggests that RAP#15 promotes pausing (compare lanes 3-6 vs. 11-14). In contrast to termination, pausing results in initial accumulation of a shorter transcript that subsequently disappears with time as the RNAP escapes pausing and continues elongation to produce a full length run-off transcript. The lentil bar indicates the run-off products. (B) Mapping the position of RAP #15. The RNA sequencing ladder was prepared as described in the Examples section. The obtained transcripts were separated on 6% sequencing gel. Transcription reaction stopped after 40 seconds of chasing (see FIG. 4A, lane 5) was used as a reference and is labeled as “40 s”. (C) Quantification of single-round runoff transcription shown in (A). For each reaction, RAP-containing transcript abundance was calculated based on the band intensity on gel relatively (%) to the total signal of accumulated shorter transcripts without the RAP (part under the rectangle) reached at certain time points (0 to 60 seconds).

FIG. 18. Searching for consensus pausing G₋₁₀Y₋₁G₊₁ motif in Inhibiting RAPs. (A) Enrichment analysis for the G₋₁₀Y₋₁G₊₁ motif in the sense RAPs. Elements from 5′UTR, 3′UTR and intragenic categories comprise a group of sense RAPs. The red line depicts the number of sense RAPs containing the motif. The histogram shows the respective number for 10,000 randomly shuffled sets of RAPs. Number, length and strand of the RAPs are kept constant during shuffling, only the genomic position is changed. The dashed line shows the 95% quantile of the distribution, indicating that the G₋₁₀Y₋₁G₊₁ motif is enriched in our set of sense RAPs. (B) Enrichment analysis for the G₋₁₀Y₋₁G₊₁ motif in the anti-sense RAPs. Description is same as in (A). For the anti-sense RAPs no enrichment of the G₋₁₀Y₋₁G₊₁ motif could be observed.

FIG. 19. Nucleotide distribution within tested inhibitory RAPs. Values represent means±SD within a group of sequences.

FIG. 20. Sequence logo presentation of identified consensus sequences for inhibitory RNA-polymerase binding aptamers. Prediction was performed with The MEME Suite 4.10.0: Motif-based sequence analysis tools. Reported activating RAP sequences were submitted to MEME program with the following parameters: “site distribution”—one per sequence; “search given strand only”. The E-values are given in the Figure. For details see Example 2.

FIG. 21. Evaluating the effect of RAP #5713 overexpression in trans.

(A) Design of the experiments for evaluating effect of an activating RAP (RAP ID #5713; SEQ ID NO:2) on transcription in trans. E. coli cells were re-transformed with two compatible plasmids carrying different resistance markers (shown schematically as circles). The first plasmid (pMW) encodes GFP reporter gene with one of two well characterized terminators; i.e. either carrying the rho-dependent rrnB (pWM_rrnB) or the rut intrinsic terminator (pWM_rut). The second plasmid carries an arabinose-inducible promoter for overexpression of RAP #5713 in a form of bacterial small (sRNA). (B) Left panel: sRNA design, sRNA without (sRNA) and with (RAP_sRNA). The scaffold of DsrA, RprA and their hybrid was used to create 3 different RAP-containing sRNAs (RAP_sRNA1, RAP_sRNA2 and RAP_sRNA3. respectively) by replacing the base-pairing region with the corresponding RAP sequence. The location of a probe for RAP-containing sRNA detection is depicted as an elliptic bar. Right panel: Northern blot analysis of total RNA from transformed E. coli cells using a probe hybridizing to RAP #5713. The Northern blot shows the accumulation of RAP#5713-containing full-length stable transcripts (thicker (major) band indicated with an arrow), overexpressed from RAP_sRNA (pBAD30-based) plasmid upon induction with L-arabinose (1%). Probing for the 5S RNA was performed for the loading control.

(C)-(D) The results of co-transformation with two plasmids: pWM with intrinsic rrnB terminator (C) or rut site (D) (schematically shown as hairpin or rectangle, respectively) upstream of the reporter GFP. Further, pBAD30-based RAP_sRNA plasmid for overxpression of RAP-containing sRNAs (upper panel). “P” (grey triangle) indicates the location of constitutive bacterial RNAP promoter. TSS: transcription start site; RBS: ribosomal binding site. Middle and lower panels: E. coli cells co-transformed with described GFP reporter plasmid and different plasmids for RAP_sRNA overexpression were grown on LB agar plates followed by fluorescence intensity measurements (middle panel—GFP mode). The same plate was also captured under visible light showing bacterial density of different strains (lower panel—light mode).

FIG. 22: GLAM2 output of the motif (SEQ ID NO:138) for transcription enhancing RAPs (of yeast and human origin), regulating transcription in E. coli.

FIG. 23: Inhibiting RAPs enable Rho to act within the translated region. (A) Schematic of the reporter constructs used to test the inhibiting effect of RAP15 (SEQ ID NO:25) within a translated region. Upper panel: RAP15 (blue triangle) in its endogenous context (first 168 nt of nadD gene) was fused with GFP reporter protein. Lower panel: control construct with reverse complement sequence of RAP15-rev #15 (green triangle). “P” (grey triangle) indicates the promoter location. TSS: transcription start site; RBS: ribosomal binding site. (B)-(C) E. coli GFP plate assay. Left panels: E. coli cells transformed with nad(RAP#15)-GFP or nad(rev#15)-GFP reporter plasmids were grown on LB agar plates (B): without bicyclomycin (BCM), (C): in the presence of BCM) followed by fluorescence intensity measurements (GFP mode). Right panels: corresponding plates were captured under visible light showing the bacterial density of strains with translational fusions (Light mode).

(D) E. coli growth experiment with real-time fluorescence detection. Cells transformed with nad(RAP15)-GFP or nad(rev #15)-GFP reporter constructs (shown in blue and green) were grown in LB media with simultaneous cell density (upper panel) and GFP intensities (lower panel) measurements. Grey vertical line separates exponential and stationary phases. At each time point values represent means±SD, n=3.

(E) A model for RAP-mediated transcription termination. (1) Coupling between the leading ribosome and RNAP leaves no room for Rho to load on the nascent transcript (S. Proshkin, A. R. Rahmouni, A. Mironov, E. Nudler, Science. 328, 504-8 (2010), B. M. Burmann et al., Science. 328, 501-4 (2010)); (2) An inhibitory RAP emerging from the RNAP exit channel interacts with RNAP, resulting in ribosome pausing and RNA looping; (3) Naked unstructured RNA becomes available for Rho to load and terminate transcription.

### DETAILED DESCRIPTION OF THE INVENTION

The inventors interestingly found that the short RNA-polymerase binding aptamers interacting with RNA-polymerases with high affinity regulate expression of the DNA-sequences they are encoded on. Thereby a completely new tool is provided to regulate RNA-polymerases and thereby a tool for regulating expression of DNA-encoded molecules, like RNAs and/or Proteins, in a host cell. Hence, the present invention in particular relates to the use of the RNA-polymerase binding aptamers as disclosed herein for the use in regulating expression of a sequence of interest. The inventors found that the inventive RNA-polymerase binding aptamers surprisingly act in cis, i.e. they regulate the expression of nucleic acid they are encoded on and transcribed with, e.g. of the DNA strand they are encoded on. Hence, the RNA-polymerase binding aptamer according to the invention in all its embodiments and aspects is preferably used in cis. Be used in cis in context of the present invention means that the sequence encoding the RNA-polymerase binding aptamer is positioned such that it is transcribed together with the sequence to be expressed, i.e. it forms part of the same RNA when transcribed. Generally, this means that the sequence encoding the RNA-polymerase binding aptamer is positioned downstream of the transcription starting site and upstream of a transcription terminating signal of the constructs of the present invention.

The RNA-polymerase binding aptamer according to the present invention is preferably RNA. Further preferred, said RNA-polymerase binding aptamer may be encoded on another nucleic acid, e.g. and preferably DNA. Hence, the invention also relates to a DNA molecule comprising a sequence encoding said RNA-polymerase binding aptamer.

Methods to determine the degree of affinity are well established and known at the RNA biochemistry field. In particular methods are known to determine the affinity between a nucleic acid molecule, like RNA or DNA, and a protein or polypeptide, like RNA-Polymerase. Affinity of interactions can be expressed in terms of dissociation constant. The dissociation constant is commonly used to describe the affinity between a ligand (“L”; in the present case the RNA-polymerase binding aptamer) and a protein (“P”; in the present case RNA-polymerase). In other words, the dissociation constant expresses how tightly a ligand binds to a particular protein. Ligand-protein affinities are influenced by non-covalent intermolecular interactions between the two molecules such as hydrogen bonding, electrostatic interactions, hydrophobic and Van der Waals forces. The formation of a ligand-protein complex (“C”) can be described by a two-state process

C [symbol] P+L

The respective dissociation constant (K_{D}) can be expressed as

$${K}_{D} = \frac{[ P ] + [ L ]}{[ C ]}$$

[P], [L] and [C] represent molar concentrations of the protein, ligand and complex, respectively. The unit of the dissociation constant is molarity (M) and corresponds to the concentration of ligand at which the binding site on a particular protein is half occupied, i.e. the concentration of ligand, at which the concentration of protein with ligand bound [C], equals the concentration of protein with no ligand bound [P]. The smaller the dissociation constant, the more tightly bound the ligand is, or the higher the affinity between ligand and protein. For example, a ligand with a nanomolar (nM) dissociation constant binds more tightly to a particular protein than a ligand with a micromolar (μM) dissociation constant. The determination of K_{D} for an RNA-polymerase aptamer according to the present invention may be performed with methods well known to the skilled person. For example the RNA-polymerase aptamer (being RNA) could be analysed by RNA electrophoretic mobility shift assays (RNA EMSAs). In this case EMSA experiments are performed with an in vitro transcribed RNA aptamer and purified RNA polymerase. To determine the K_{D} values one several binding reactions are performed with a fixed concentration of radioactively labeled RNA and increasing concentrations of the protein of choice (RNAP). The detailed step-by-step protocol is provided in “Handbook of RNA Biochemistry” (2014), Edited by R. K. Hartmann, A. Bindereif, A. Schön, E. Westhof. Volume 2, pp. 975-983, Wiley, which is incorporated herein by reference. The inventors have ascertained that the aptamers bind to the RNA-polymerase with the desired high affinity by repeatedly applying the SELEX procedure (7 cycles of genomic SELEX resulting in dissociation constant of about 10 nM). Hence, the skilled person may perform EMSA experiments for the RNA aptamer of choice (e.g. in vitro transcribed) in order to determine the exact Kd if required. Such procedure is for example described in N. Windbichler, F. von Pelchrzim, O. Mayer, E. Csaszar, R. Schroeder, RNA Biol. 5, 30-40, and genomic SELEX is described in C. Lorenz, F. von Pelchrzim, R. Schroeder, Nat. Protoc. 1, 2204-12 (2006), which are both incorporated herein by reference.

The RNA-polymerase binding aptamer according to the present invention binds the RNA-Polymerase with a K_{D} of 50 nM or less, more preferably with a K_{D} of 10 nM or less. The RNA-polymerase binding aptamer according to the present invention preferably binds the RNA-polymerase with a K_{D} of 50 nM or less, more preferably with a K_{D} of 10 nM or less as a single stranded RNA. It will be understood by the skilled person that the RNA-polymerase binding aptamer according to the present invention is able to bind the RNA-polymerase as single stranded RNA, i.e. without the need for being hybridized to other nucleic acid, such as DNA or another RNA strand. Hence, preferably the RNA-polymerase binding aptamer according to the present invention binds the RNA-polymerase according to the present invention as single stranded RNA with a K_{D} of 50 nM or less, more preferably with a K_{D} of 10 nM or less. The RNA-polymerase binding aptamer according to the present invention preferably binds the RNA-polymerase with a K_{D} of 50 nM or less, more preferably with a K_{D} of 10 nM or less as a single stranded RNA.

The inventors found RNA-polymerase binding aptamers binding RNA-polymerase from Escherichia coli. However, as will be apparent to the skilled person, it is plausible that the invention is likewise applicable for other RNA-polymerases, e.g. RNA-polymerases of other prokaryotic or eukaryotic organisms. The skilled person will acknowledge that applicable also to the eukaryotic RNA-polymerases, as they share high structural similarities with prokaryotic in general and bacterial RNA-polymerases in particular. Furthermore, the inventors have proven in the enclosed Example section that also RNA-polymerase of eukaryotic organisms binding aptamers are a tool for regulating the expression of a sequence to be expressed. Hence, in one embodiment of the present invention said RNA-polymerase binding aptamer is binding to a RNA-polymerase selected from the group consisting of a prokaryotic RNA-polymerase, and a eukaryotic RNA-polymerase, particularly preferred the RNA-polymerase is a prokaryotic RNA-polymerase, more preferably RNA-polymerase from E. coli. Eukaryotic RNA polymerases II and III are the most closely related to bacterial RNA-polymerases (see e.g. Ebright R H (2000), “RNA polymerase: structural similarities between bacterial RNA polymerase and eukaryotic RNA polymerase II”. J. Mol. Biol. 304(5): 687-698; and Nielsen S, Yuzenkova Y, Zenkin N. (2013), Mechanism of eukaryotic RNA polymerase III transcription termination Science, 340(6140):1577-1580) for which the Examples show data. Hence, the eukaryotic RNA-polymerase according to the present invention is preferably selected from the group consisting of eukaryotic RNA-polymerase II, and eukaryotic RNA-polymerase III, particularly preferred eukaryotic RNA-polymerase II, such as from yeast or mammalian origin.

The RNA-polymerase is preferably of the organism in which the expression by RNA-polymerase shall be regulated, e.g. if the RNA-polymerase binding aptamer is intended to regulate expression of a sequence to be expressed in yeast it preferably binds RNA-polymerase of yeast or mammalian cells, such as human cells, non-human primate cells and rodent cells, including hamster cells such as BHK21, BHK, CHO, DG44, or derivatives/descendants of these cell lines. Also suitable are myeloma cells from the mouse, preferably NS0 and Sp2/0-AG14 cells and human cell lines such as HEK293 or PER.C6, as well as derivatives/descendants of these mouse and human cell line, e.g. RNA-polymerase II or RNA-polymerase III from yeast or these mammalian cell lines. If the RNA-polymerase shall be regulated in a bacterium, e.g. E. coli, the RNA-polymerase the aptamer is binding to is the RNA-polymerase of this organism, e.g. E. coli.

In one embodiment, the prokaryotic RNA-polymerase is a bacterial RNA-polymerase, more preferably a bacterial RNA-polymerase derived from gram negative bacteria or gram positive bacteria, most preferably gram negative bacteria. In one particular preferred embodiment the RNA-polymerase according to the present invention is of Escherichia coli. It will be understood by the skilled artisan that the RNA-polymerase binding aptamer may interact with any subunit of the RNA-polymerases according to the present invention. However, in a preferred embodiment of the invention the RNA-polymerase binding aptamer binds the RNA-Polymerase holoenzyme, preferably with a K_{D} of 50 nM or less, more preferably with a K_{D} of 10 nM or less. For example, such RNA-polymerase holoenzyme of the RNA-polymerase of a bacterium, e.g. E. coli, may comprise or consist of RNA-polymerase core and the sigma 70.

In one embodiment, the eukaryotic RNA-polymerase is a RNA-polymerase of fungal or mammalian origin. In one preferred embodiment the RNA-polymerase according to the present invention is of yeast or a mammalian cell line such as human cells, non-human primate cells and rodent cells, including hamster cells such as BHK21, BHK, CHO, DG44, or derivatives/descendants of these cell lines. Also suitable are myeloma cells from the mouse, preferably NS0 and Sp2/0-AG14 cells and human cell lines such as HEK293 or PER.C6, as well as derivatives/descendants of these mouse and human cell. It will be understood by the skilled artisan that the RNA-polymerase binding aptamer may interact with any subunit of the RNA-polymerases according to the present invention. However, in a preferred embodiment of the invention the RNA-polymerase binding aptamer binds a eukaryotic RNA-Polymerase II, preferably with a K_{D} of 50 nM or less, more preferably with a K_{D} of 10 nM or less. For example, such RNA-polymerase II is from yeast or a mammalian cell line, such as human cells or the cell lines listed above.

The RNA-polymerase binding aptamers according to the present invention are short. Nowadays, bacteriophage RNA sequences are known that regulate expression DNA sequences. However, such sequences are rather long and require particular and distinct secondary structures. Examples from phages like HK022 exist where the phage RNA sequences have a length of significantly more than 60 nt and show very particular secondary structures with two long stems. However, the present invention is based on the finding that shorter RNA aptamers, e.g. having a length of 23 to 50 nt, are able to regulate RNA-Polymerase activity when they are directly interacting with said RNA-Polymerase. Hence, the RNA-polymerase binding aptamer according to the present invention have a length of 15 to 60 nt, preferably 20 to 50 nt, more preferably 23 to 50 nt.

The need for structural complexity for the previously described regulatory bacteriophage RNAs make them prone to misfolding dependent on the sequence they are attached to. However, the inventors now found that the natural bacterial or eukaryotic RNA-polymerase binding aptamer according to the present invention supersedes the need for distinct secondary structures. Without being bound by theory, it is believed that this property is conveyed by the high affinity to RNA-polymerases. Hence, in one embodiment the RNA-polymerase binding aptamer according to the present invention do not posses a defined secondary structure, preferably as predicted by RNfold Software (Viena RNA Package, RNAfold—http://rna.tbi.univie.ac.at/cgi-bin/RNAfold.cgi (R. Lorenz, S. H. Bernhart, C. Hoener zu Siederdissen, H. Tafer, C. Flamm, P. F. Stadler and I. L. Hofacker (2011), “ViennaRNA Package 2.0”, Algorithms for Molecular Biology: 6:26; and I. L. Hofacker, W. Fontana, P. F. Stadler, S. Bonhoeffer, M. Tacker, P. Schuster (1994), “Fast Folding and Comparison of RNA Secondary Structures”, Monatshefte f. Chemie: 125, pp 167-188) and/or Mfold (http://mfold.rna.albany.edu/?q=mfold (Zuker, M (2003), Mfold web server for nucleic acid folding and hybridization prediction, Nucl Acid Res, 31:3406-3415)).

The inventors have identified several preferred aptamers. Hence, the RNA-polymerase binding aptamer according to the present invention is in one embodiment encoded by a sequence having at least 80% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a further embodiment the RNA-polymerase binding aptamer according to the present invention is encoded by a sequence having at least 90% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In yet a further preferred embodiment the RNA-polymerase binding aptamer according to the present invention is encoded by a sequence having at least 95% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a particular preferred embodiment the RNA-polymerase binding aptamer according to the present invention is encoded by a sequence having at least 99% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a further particular embodiment the RNA-polymerase binding aptamer according to the present invention is encoded by a sequence having a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:18, SEQ ID NO:19, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. A particular preferred RNA-polymerase binding aptamer according to the present invention is encoded by the sequence of SEQ ID NO:2 or a sequence having at least 80% identity thereto, preferably at least 90% identity, more preferably at least 95% identity, yet more preferred at least 97% identity, even more preferred at least 99% identity. The invention also relates to a nucleic acid, e.g. RNA or DNA, hybridizing to any of these sequences under stringent conditions.

The inventors unexpectedly found that expression of DNA molecules can be regulated by introducing a sequence encoding a RNA-polymerase binding aptamer according to the present invention. The above outlined properties of the RNA-polymerase binding aptamer according to the present invention are properties of the RNA-molecule. Hence, in a particular preferred embodiment the RNA-polymerase binding aptamer according to the present invention is RNA.

The sequences given herein show the DNA-sequence encoding the preferred aptamers. Hence, whenever a DNA-sequence or an encoding sequence is referred to, the sequence is preferably the one given under the respective SEQ ID NO. By being encoded by a certain SEQ ID NO: it is meant that the RNA aptamer has the sequence shown in the referred sequence except for the nucleobase thymin (T) being exchanged by an uracil (U) and the molecule being an RNA molecule rather than a DNA molecule.

However, as the skilled person will readily acknowledge, these aptamers are particularly useful to control and/or regulate expression of a DNA molecule, e.g. a DNA molecule the aptamer is encoded on. The inventors found that the aptamer is particularly useful for regulating expression of RNA from DNA in cis. Hence, the present invention provides for DNA molecules comprising a sequence encoding the RNA-polymerase binding aptamer according to the present invention and allows thereby regulation of the expression of and from said DNA molecule. The invention, hence, relates to a DNA molecule encoding an RNA-polymerase binding aptamer according to the present invention.

The DNA molecule preferably also comprises a sequence to be expressed. Such sequence to be expressed according to the present invention is preferably a nucleotide sequence, particularly preferred a DNA sequence, encoding a RNA molecule or protein of interest. For example the encoded molecule of interest may be a regulatory RNA or a protein or polypeptide. The skilled person will instantly acknowledge that the invention is not limited to a particular type of sequence to be expressed, as the inventors have shown that expression is regulated and influenced through the RNA-polymerase binding aptamers on both levels, the transcription product level (e.g. mRNA) as well as on the subsequent translation product level (e.g. a protein or polypeptide). Nevertheless, DNA sequences are particular preferred sequences to be expressed, preferably encoding a RNA molecule or protein (polypeptide) of interest.

Several types of regulatory RNA-elements are known, for example some can down-regulate gene expression by being complementary to a part of an mRNA or a gene's DNA. MicroRNAs (miRNA; 21-22 nt) are found in eukaryotes and act through RNA interference (RNAi), where an effector complex of miRNA and enzymes can cleave complementary mRNA, block the mRNA from being translated, or accelerate its degradation. While small interfering RNAs (siRNA; 20-25 nt) are often produced by breakdown of viral RNA, there are also endogenous sources of siRNAs. siRNAs act through RNA interference in a fashion similar to miRNAs. Some miRNAs and siRNAs can cause genes they target to be methylated, thereby decreasing or increasing transcription of those genes. Animals have Piwi-interacting RNAs (piRNA; 29-30 nt) that are active in germline cells and are thought to be a defense against transposons and play a role in gametogenesis. Many prokaryotes have CRISPR RNAs, a regulatory system similar to RNA interference. Antisense RNAs are widespread; most downregulate a gene, but a few are activators of transcription. One way antisense RNA can act is by binding to an mRNA, forming double-stranded RNA that is enzymatically degraded. There are many long non-coding RNAs that regulate genes in eukaryotes, one such RNA is Xist, which coats one X chromosome in female mammals and inactivates it.

An mRNA may further contain regulatory elements itself, such as riboswitches, in the 5′ untranslated region or 3′ untranslated region; these cis-regulatory elements regulate the activity of that mRNA. The untranslated regions can also contain elements that regulate other genes. Hence, in one embodiment of the present invention the DNA-molecule comprises a sequence encoding for one or more further regulatory RNA-elements.

The DNA-molecule or expression cassette or vector, infra, can further comprise expression control sequences operably linked to said sequence to be expressed. These expression control sequences may be suited to ensure transcription and synthesis of RNA, like translatable RNA, in bacteria; such as E. coli; or eukaryotes; such as fungi or mammalian cell lines.

In a preferred embodiment of the present invention the sequence to be expressed encodes a polypeptide or protein of interest, e.g. a protein which is to be expressed.

“Expression” refers to transcription and translation occurring within a host cell. The level of expression of a DNA molecule in a host cell may be determined on the basis of either the amount of corresponding mRNA that is present within the cell or the amount of the DNA-encoded protein produced by the host cells. Further detail for the term “expression” within the context of the present invention can be obtained via a review of Sambrook et al. (2012), “Molecular Cloning: A Laboratory Manual”, 4^{th} edition, Cold Spring Harbor Laboratory Press, ISBN: 978-1-936113-42-2, which is incorporated herein by reference.

The inventors found that the sequence encoding the RNA-polymerase binding aptamer according to the present invention may be located at differing positions within a DNA-molecule in order to regulate its expression. However, in a particular preferred embodiment the sequence encoding the RNA-polymerase binding aptamer according to the present invention is operatively linked to a promoter, preferably located downstream of a promoter. This is particularly preferred, as the results presented show that the effect achieved by the invention is present in particular if the aptamer is located downstream of the promoter and thus is being transcribed.

The DNA-molecule according to the present invention at least comprises a sequence encoding the RNA-polymerase binding aptamer according to the present invention. It may contain further elements as outlined herein in great detail. However, these elements may themselves not have any functional properties, like being a promoter, transcription start site, being a sequence to be expressed. For example, the DNA molecule according to the present invention may also be used for integration purposes into the genome of a host cell or organism. In such case one or more sequences flanking the RNA-polymerase binding aptamer encoding sequences may be sufficient to trigger integration into the host's genome, preferably site specific. The mechanism used for integration may be for instance homologous recombination, non-specific incorporation of viral DNA throughout the host genome (Youngsuk et al., “Current Advances in Retroviral Gene Therapy”, Curr Gene Ther. 2011 June; 11(3): 218-228), adaptation of CRISP/Cas9 system (Harrison et al, “A CRISPR view of development”, 10.1101/gad.248252.114 Genes & Dev. 2014. 28: 1859-1872), etc.

The sequence encoding the RNA-polymerase binding aptamer according to the present invention is preferably operatively linked to a promoter on the DNA molecule or vector according to present invention to regulate expression from said promoter. The sequence encoding the RNA-polymerase binding aptamer according to the present invention is another embodiment is positioned on the DNA-molecule according to the present invention to operatively link said sequence to a promoter after integration of the DNA-molecule or fragments thereof in a nucleic acid, e.g. a host's genome, to regulate expression from said promoter.

The term “operatively linked” or “operably linked”, as used in connection with the present invention, refers to a linkage between one or more expression control sequences and/or the coding region in the polynucleotide to be expressed in such a way that expression is achieved under conditions compatible with the expression control sequence, e.g. so that the transcription enhancing RNA-polymerase binding aptamer increases expression of a sequence, or so that a transcription inhibiting RNA-polymerase binding aptamer inhibits or decreases expression of a sequence.

In a particular preferred embodiment the sequence to be expressed codes for a polypeptide or protein of interest and the RNA-polymerase binding aptamer is located in the 5′UTR region of the sequence encoding said polypeptide or protein of interest.

The DNA-molecule according to the present invention may also comprise an (entire) expression cassette. An expression cassette may be a part of a vector DNA used for cloning and transformation. The expression cassette directs the cell's machinery to make RNA and protein. An expression cassette is composed of one or more genes and the sequences controlling their expression. An expression cassette usually comprises: a promoter sequence, an open reading frame, and optionally a 3′-untranslated region that, in eukaryotes, usually contains a polyadenylation site. As outlined in great detail herein, the invention provides for a novel mechanism to regulate expression of DNA sequences, e.g. from an expression cassette. Hence, the present invention also relates to a DNA-molecule comprising an expression cassette, said expression cassette comprising a sequence encoding an RNA-polymerase binding aptamer according to the present invention, a promoter, and an open reading frame encoding a protein to be expressed, preferably the sequences encoding the aptamer, the promoter and the open reading frame are operatively linked within said expression cassette on said DNA-molecule. The expression cassette may further comprise a 3′untranslated region. The inventors have shown that the position of the sequence encoding the aptamer is not crucial as long as it is transcribed. Hence, in a preferred embodiment of the present invention the DNA-molecule according to the present invention comprises an expression cassette comprising a promoter, an open reading frame encoding a protein to be expressed, and a sequence encoding a RNA-polymerase binding aptamer according to the present invention, wherein the open reading frame and the sequence encoding said RNA-polymerase binding aptamer are positioned downstream of said promoter. The skilled person will acknowledge that the sequence encoding said aptamer may be positioned between the promoter and the open reading frame (e.g. in the 5′-untranslated region) or may be positioned within the open reading frame. The skilled person will be able to redesign coding sequences accordingly. However, in a preferred embodiment of the DNA-molecule comprising said expression cassette according to the present invention the sequence encoding the RNA-polymerase binding aptamer according to the present invention is located downstream of the promoter and upstream of the open reading frame of the protein to be expressed, i.e. in the 5′-untranslated region, particularly preferred downstream of the transcription start site of the promoter and the translation initiation site of the open reading from. Furthermore, in a preferred embodiment said sequence encoding a RNA-polymerase binding aptamer according to the present invention encodes two or more, preferably three or more consecutive aptamers, e.g. as tandem repeats, optionally the aptamers being separated through linker sequences. Preferably, the RNA-polymerase binding aptamer according to the invention is encoded downstream of the promoter and upstream of the sequence to be expressed is an activating RNA-polymerase binding aptamer according to the invention. For increasing the expression, this RNA-polymerase binding aptamer is preferably an activating RNA-polymerase binding aptamer according to the invention. This activating construct may further be enhanced by introducing the reverse complement of a sequence encoding an inhibiting RNA-polymerase binding aptamer according to the invention at the 3′ end of the expression cassette, preferably in the 3′ untranslated region (also referred to as 3′UTR). Without being bound by theory that this inhibiting aptamer represses unwanted expression from the complement strand (e.g. during pervasive transcription) and thereby avoids blocking of the expression from the desired strand by opposing transcription.

As will be readily understood, the present invention is not limited to a specific promoter or type or sequence to be expressed, e.g. a specific protein or RNA. Expression cassettes are designed for modular cloning of (protein-encoding) sequences so that the same cassette can easily be altered to make different proteins. To this end, an expression cassette may be provided which does not contain an open reading frame but a multiple cloning site allowing the insertion of a sequence to be expressed, e.g. a sequence encoding a protein of interest or a RNA of interest. Hence, the invention further relates to DNA-molecule comprising an expression cassette, said expression cassette comprising a sequence encoding an RNA-polymerase binding aptamer according to the present invention, a promoter, and an multiple cloning site for inserting a sequence to be expressed, preferably said sequence encoding the aptamer, said promoter and said multiple cloning site are operatively linked. In a preferred embodiment of the present invention the DNA-molecule according to the present invention comprises an expression cassette comprising a promoter, a multiple cloning site for inserting a sequence to be expressed, and a sequence encoding a RNA-polymerase binding aptamer according to the present invention, wherein said multiple cloning site and said sequence encoding said RNA-polymerase binding aptamer are positioned downstream of said promoter. In a preferred embodiment of the DNA-molecule comprising said expression cassette according to the present invention the sequence encoding the RNA-polymerase binding aptamer according to the present invention is located downstream of the promoter and upstream of the multiple cloning site for inserting a sequence to be expressed.

The expression cassette may also contain more than one sequence to be expressed, e.g. being a polycistronic expression cassette. That means that transcription is initiated from a single promoter. The mRNA found in bacteria is mainly polycistronic. This means that a single bacterial mRNA strand can be translated into several different proteins. This will occur if spacers separate the different proteins, and each spacer has to have a translation initiation site (ribosome binding site, Shine-Dalgarno sequence) located upstream of the respective start codon. The transcribed mRNA than comprises a plurality of sequences to be expressed, e.g. a two or more open reading frames encoding for a protein. The skilled person will acknowledge that the RNA-polymerase binding aptamers according to the present invention are also suited to enhance expression of such polycistronic transcripts. In one embodiment the expression cassette according to the present invention comprises a promoter, two or more open reading frames transcribed from said promoter, wherein the expression cassette comprises two or more sequences encoding a RNA-polymerase binding aptamer according to the present invention, wherein said sequences encoding said aptamer are operatively linked to said promoter and said open reading frames to allow expression of all open reading frames. In a preferred embodiment each open reading frame is operatively linked to one or more sequences encoding a RNA-polymerase binding aptamer according to the present invention, particularly preferred the two or open reading frames are separated by a sequence comprising one or more sequences encoding a RNA binding aptamer according to the present invention, and a translation initiation site operatively linked thereto, preferably a ribosomal binding site downstream of said sequence encoding the aptamer. In a preferred embodiment the two or more sequences encode transcription enhancing RNA-polymerase binding aptamers according to the present invention. Each open reading frame may furthermore been linked to a translation initiation site (e.g. a ribosomal binding site, Shine-Dalgarno sequence). In a preferred embodiment the expression cassette hence comprises a promoter, and two or more open reading frames, wherein the expression cassette comprises upstream of each open reading frame a sequence encoding a RNA-polymerase binding aptamer according to the present invention, preferably a transcription enhancing RNA-polymerase binding aptamer according to the present invention, and a translation initiation site. The open reading frames may encode all the same protein or may encode for different proteins.

Different expression cassettes can be transformed into different organisms including bacteria, yeast, plants, and mammalian cells as long as the correct regulatory sequences are used.

Particularly preferred are expression cassettes for bacteria, preferably E. coli. A promoter is a region of DNA that initiates transcription of the downstream sequence. Promoters are located near the transcription start sites of genes, on the same strand and upstream on the DNA (towards the 5′ region of the sense strand). Promoters can be of differing sized, e.g. about 100-1000 nt long. For the transcription to take place, the RNA polymerase binds to the promoter sequence or a sequence nearby. Promoters may contain specific DNA sequences such as response elements that provide a secure initial binding site for RNA polymerase and for proteins called transcription factors that recruit RNA polymerase. These transcription factors have specific activator or repressor sequences of corresponding nucleotides that attach to specific promoters and regulate gene expression. In bacteria the promoter is recognized by RNA polymerase and an associated sigma factor, which in turn are often brought to the promoter DNA by an activator protein's binding to its own DNA binding site nearby. In eukaryotes the process of transcription initiation is more complex, and at least seven different factors are necessary for the binding of an RNA polymerase II to the promoter. In bacteria, the promoter contains two short sequence elements approximately −10 and −35 nucleotides upstream from the transcription start site. The sequence at −10 (the −10 element) has the consensus sequence TATAAT. The sequence at −35 (the −35 element) has the consensus sequence TTGACA. These consensus sequences, while conserved on average, are not found intact in most promoters. On average, only 3 to 4 of the 6 base pairs in each consensus sequence are found in any given promoter. Few natural promoters have been identified to date that possess intact consensus sequences at both the −10 and −35; artificial promoters with complete conservation of the −10 and −35 elements have been found to transcribe at lower frequencies than those with a few mismatches with the consensus. The optimal spacing between the −35 and −10 sequences is 17 bp. It should be noted that the above promoter sequences are recognized only by RNA polymerase holoenzyme containing sigma-70. RNA polymerase holoenzymes containing other sigma factors recognize different core promoter sequences and are likewise covered by the present invention. As has been shown by the inventors, the superior technical effects of the RNA-polymerase binding aptamers according to the present invention are achieved independent of the promoter used. As will be understood by the skilled person, the promoter shall be chosen according to the intended host cell, i.e. when the host cell is a bacterium a bacterial promoter shall be used, and, likewise, in case the host cell is a eukaryotic cell a eukaryotic promoter shall be used. Preferred promoters are promoters being active in E. coli, e.g. E. coli promoters. Preferred promoters according to the present invention include constitutive and inducible promoters. Promoters for use in connection with the DNA molecule and the expression cassette, respectively, may be homologous or heterologous with regard to its origin and/or with regard to the gene or sequence to be expressed. Suitable promoters are for instance promoters which lend themselves to constitutive expression. However, promoters which are only activated at a point in time determined by external influences can also be used. Artificial and/or chemically inducible promoters may be used in this context. Inducible promoters comprise arabinose-, glucose-, IPTG-, alcohol-, metal-inducible promoters.

One example of a constitutive promoter is the one used in pWM3110 (see Example section). In one embodiment the expression cassette according to the present invention comprises a promoter having at least 80% identity to SEQ ID NO:66, preferably at least 90% identity to SEQ ID NO:66, more preferably at least 95% identity to SEQ ID NO:66, yet more preferred at least 99% identity to SEQ ID NO:66. In a particular preferred embodiment the expression cassette according to the present invention comprises a promoter having the sequence of SEQ ID NO:66.

One example of an inducible promoter is an arabinose inducible promoter, e.g. the arabinose-inducible promoter as used in the examples and having the sequence of SEQ ID NO:67. Hence, in one embodiment the expression cassette according to the present invention comprises a promoter having at least 80% identity to SEQ ID NO:67, preferably at least 90% identity to SEQ ID NO:67, more preferably at least 95% identity to SEQ ID NO:67, yet more preferred at least 99% identity to SEQ ID NO:67. In a particular preferred embodiment the expression cassette according to the present invention comprises a promoter having the sequence of SEQ ID NO:67.

It has been shown herein, that the RNA-polymerase binding aptamers are able to regulate transcription of a transcript comprising the aptamer. As outlined herein, the sequence encoding the RNA-polymerase binding aptamer according to the present invention is preferably operatively linked to the promoter sequence and the transcription starting site of the promoter. It is preferably positioned downstream of the promoter and the transcription starting site on the DNA-molecule according to the present invention. The distance of the RNA-polymerase binding aptamer encoding sequence to the transcription starting site is believed not being crucial. However, with certain distances superior effects in terms of regulating the transcription may be observed. Hence, in one embodiment the sequence encoding the RNA-polymerase binding aptamer according to the present invention is preferably positioned within a distance of 10 to 1000 nt downstream of the transcription starting site, preferably within a distance of 50 to 500 nt downstream of the transcription starting site, more preferred within a distance of 60 to 250 nt downstream of the transcription starting site, particularly preferred within a distance of 80 to 103 nt downstream of the transcription starting site.

The sequence encoding the RNA-polymerase binding aptamer according to the present invention is preferably also operatively linked to the sequence to be expressed, e.g. an open reading frame, or a multiple cloning site. The distance of the RNA-polymerase binding aptamer encoding sequence to the sequence to be expressed or the multiple cloning site is not crucial. However, with certain distances superior effects in terms of regulating the transcription and expression may be observed. Hence, in one embodiment the sequence encoding the RNA-polymerase binding aptamer according to the present invention is preferably positioned within a distance of 10 to 1000 nt upstream of the sequence to be expressed or the multiple cloning site, preferably within a distance of 50 to 500 nt upstream of the sequence to be expressed or the multiple cloning site, more preferred within a distance of 60 to 250 nt upstream of the sequence to be expressed or multiple cloning site, particularly preferred within a distance of 70 to 108 nt upstream of the sequence to be expressed or multiple cloning site.

Any combination of the above mentioned preferred distances may be used, e.g. a distance of 10 to 1000 nt downstream of the transcription starting site and a distance of 10 to 1000 nt upstream of the sequence to be expressed, or distance of 50 to 500 nt downstream of the transcription starting site and a distance of 50 to 500 nt upstream of the sequence to be expressed, or the multiple cloning site, or a distance of 60 to 250 nt downstream of the transcription starting site and a distance of 60 to 250 nt upstream of the sequence to be expressed, or the multiple cloning site, or a distance of 80 to 103 nt downstream of the transcription starting site and a distance of 70 to 108 nt upstream of the sequence to be expressed, or the multiple cloning site, or any other possible combination.

The skilled person will acknowledge that the DNA molecule according to the present invention is applicable to a variety of applications. Hence, the present invention also relates to the use of a sequence encoding a RNA-polymerase binding aptamer according to the present invention for regulating expression from a DNA molecule.

In a preferred embodiment the invention relates to the use of the DNA-molecule according to the present invention for regulating expression of an endogenous sequence to be expressed or an exogenous sequence to be expressed in a host cell or organism.

The skilled person is in the position to choose the specific embodiments for the respective use and application when considering the disclosure of the present application. For instance, the DNA-molecule may comprise a part or the entire sequence to be expressed. It may for purpose of site specific integration at the proper site of the host's genome, e.g. by homologous recombination, comprise only a part of the endogenous sequence to be expressed in order to allow integration at the proper and desired site to secure proper expression. Hence, in one embodiment the DNA-molecule according to the present invention comprises one or more elements (e.g. sequences) for site specific integration into a host's genome.

The skilled person, when considering the disclosure of the present invention, is in the position to choose the proper site of integration and to design the DNA-molecule accordingly. For such design he will in particular by consider certain embodiments of the present invention as described herein in great detail, e.g. with respect to the distance between the sequence encoding the RNA-polymerase binding aptamer according to the present invention to, for example, the promoter/transcription starting site and/or the ribosome binding site and/or the start codon of a gene to be expressed. As outlined herein above, the sequence encoding the RNA-polymerase binding aptamer according to the present invention for regulating the expression of the sequence to be expressed is preferably located downstream of the transcription starting site of a promoter. The promoter, however, may be present in the DNA-molecule itself or the DNA-molecule may contain sequences for site specific integration of the construct into the host's genome to position the sequence encoding the RNA-polymerase binding aptamer downstream of an endogenous transcription start site of a promoter. Hence, in one embodiment of the present invention, the DNA-molecule according to the present invention comprises a sequence for site specific integration into a host's genome, preferably for site specific integration downstream of a promoter and/or transcription start site.

In a one embodiment the DNA-molecule comprises a for site specific integration upstream of an endogenous sequence to be expressed. In such embodiment the DNA-molecule may comprise an endogenous or exogenous promoter and/or a transcription start site, preferably upstream of the sequence encoding the RNA-polymerase binding aptamer according to the present invention.

The RNA-polymerase binding aptamer may be chosen according to the needs, e.g. an inhibiting RNA-polymerase binding aptamer according to the present invention may be employed, i.e. encoded on said DNA-molecule, if the expression of said endogenous sequence is to be downregulated. Hence, the invention relates to the use of an inhibiting RNA-polymerase binding aptamer according to the present invention or a DNA-molecule encoding the same for inhibiting the expression of an endogenous sequence within a host cell. The host cell preferably being a non-human host cell, preferably bacteria origin. The DNA-molecule encoding inhibiting RNA-polymerase binding aptamer according to the present invention is preferably inserted downstream of the promoter from which said endogenous sequence is transcribed. The invention also relates to a DNA-molecule according to the present invention for use in treating a human through gene therapy.

The skilled person will acknowledge that the DNA-molecule according to the present invention may be used for regulating the expression of an endogenous sequence to be expressed or for regulating the expression of an exogenous sequence through the RNA-polymerase binding aptamer according to the present invention by employing an endogenous promoter of the host. In case the DNA-molecule according to the present invention is used for regulating the expression of an endogenous sequence to be expressed this may be performed by using the endogenous promoter of the sequence to be expressed or by introducing an exogenous promoter to control the start of the transcription. In the latter case, the skilled artisan will understand that the DNA-molecule according to the present invention preferably comprises a promoter, preferably upstream of the sequence encoding the RNA-polymerase binding aptamer and optionally additional sequences for proper integration.

Furthermore, the skilled person will acknowledge the plurality of different uses and applications of the aptamers according to the invention. The aptamers or the DNA-molecules according to the present invention may be used for regulating expression of sequences to be expressed within a host cell or in an in vitro expression system. By application of the different aptamers the skilled person may regulate the expression with a finer tuning than known from prior art. When using gene silencing or knock out techniques to inhibit expression of a gene, e.g. for functional studies, the result is usually the complete loss of the gene product, i.e. the RNA or the encoded protein. However, for some purposes it is desirable to not completely diminish expression and/or activity of a certain gene product. For instance if the gene and/or gene product is essential for cell survival. The skilled person will instantly acknowledge that the present invention provides a superior tool allowing down-regulation of gene expression, e.g. of endogenous sequences to be expressed, without completely depleting the cell for the encoded molecule. The aptamers according to the present invention are therefore particularly applicable for scientific research, e.g. by using them to down or up regulate the expression of a sequence, e.g. encoding a molecule of interest. The skilled person will acknowledge that this is the case for prokaryotic as well as eukaryotic cells. For the latter the present invention provides for a superior alternative to gene-silencing through siRNAs and the like. Hence, the disclosed methods and uses herein as they relate on the inhibiting RNA-polymerase binding aptamers in a particular preferred embodiment are methods or uses for knock down of a sequence of interest in a host cell (e.g. a gene). Preferred methods are outlined further below.

In one embodiment of the invention the use of the DNA-molecule relates to the use for regulating the expression of an endogenous sequence to be expressed by employing the endogenous promoter of said sequence. In such embodiment the DNA-molecule preferably comprises a sequence encoding a RNA-polymerase binding aptamer and one or more sequences for site specific integration of said sequence encoding a RNA-polymerase binding aptamer downstream of said endogenous promoter and upstream of said sequence to be expressed.

“Endogenous” refers to any sequence, promoter, transcription start site, gene, open reading frame or the like that is present in the host cell or organism without insertion or application of external nucleic acid sequence, e.g. before external application of the DNA-molecule according to the present invention. An endogenous sequence is present in a cell or organism without any external manipulation, e.g. transfection or transformation of nucleic acids.

“Exogenous ” refers to any sequence, promoter, transcription start site, gene or the like that is not present in the host cell or organism or not present at the specific locus (site) of the host's genome without insertion or application of external nucleic acid sequence, e.g. the “exogenous” element is only present at the specific site of the host's genome after external application of the DNA-molecule according to the present invention.

“Sequence to be expressed” as used herein refers to a DNA sequence encoding a molecule of interest. As outlined herein, the RNA-polymerase binding aptamers according to the present invention regulate the activity of RNA-polymerases, e.g. and preferably DNA-dependent RNA-polymerases, i.e. they influence the transcription of a DNA sequence into RNA. Thereby also the yield of subsequent steps, like translation into a polypeptide, can be influenced. Hence, the “sequence to be expressed” as used herein preferably refers to a DNA sequence encoding a molecule of interest, the molecule of interest preferably being a RNA molecule or a polypeptide or protein molecule of interest.

“Host cell” or “host cells” refers to cells expressing or capable of expressing a sequence to be expressed. Host cells of the present invention express polynucleotides encoding polypeptides or RNAs having any number of uses, including biotechnological, molecular biological and clinical settings. Examples of suitable host cells in the present invention include, but are not limited to, bacterial, yeast, insect, animal and mammalian cells. Specific examples of such cells include E. coli DH5a cells and BL21(DE3), as well as various other bacterial cell sources, for example the E. coli strains: DH10b cells, XL1Blue cells, XL2Blue cells, Top10 cells, HB101 cells and DH12S cells; yeast host cells from the genera including Saccharomyces, Pichia, and Kluveromyces, animal cell lines, e.g. mammal and even human cell lines, including CHO cells, HEK293 cells, and HeLa. Preferred host cells are those using bacterial RNA-polymerase, or eukaryotic DNA-dependent RNA-polymerase II and/or III; preferably RNA-polymerase II; for expression of DNA sequences. In one particular preferred embodiment the host cell is a bacterial cell, preferably E. coli; even more preferred selected from the group of E. coli strains consisting of DH5a, BL21(DE3), DH10b cells, XL1Blue cells, XL2Blue cells, Top10 cells, HB101 cells and DH12S cells, yet more preferred DH5α, or BL21(DE3). In another preferred embodiment the host cell is a fungus, more preferably a fungus of the genus Saccharomyces, Schizosaccharomyces, Aspergillus, Trichoderma, Kluyveromyces or Pichia and even more preferably of the species Saccharomyces cerevisiae, Schizosaccharomyces pombe, Aspergillus niger, Trichoderma reesei, Kluyveromyces marxianus, Kluyveromyces lactis, Pichia pastoris, Pichia torula or Pichia utilis. In another preferred embodiment the host cell is a eukaryotic host cell selected from the group of yeasts (e.g. Saccharomyces cervisiae), fungal host cells, insect host cells, mammalian host cells such as primate, human or rodent host cells, including CHO or COS cells, HEK293 cells, and HeLa The skilled artisan will acknowledge that the host cell may comprise further modifications, like additional genes, mutations or deletions, e.g. to enhance expression efficiency.

Even though the identified aptamers may play a role in gene expression in nature, one embodiment of the present invention is the provision of a DNA-molecule, a vector, and/or a host cell for regulated expression of a sequence of interest (a sequence to be expressed). Hence, in a preferred embodiment the subject-matter of the present invention relates to recombinant products, like recombinant RNA- or DNA-molecules, vectors and/or host cells. Hence, the invention preferably excludes naturally occurring host cells as found in nature expressing employing the regulatory mechanism of RNA-polymerase binding aptamers for the sequence to be expressed. Instead, the host cell of the present invention and employed in a method or use of the present invention is preferably a non-naturally occurring host cell, whether it has been genetically modified to express (including overexpression and repression/inhibition of expression) a sequence to be expressed in a context (regulatory mechanism through employment of the aptamers according to the present invention) not normally existing in its genome or whether it has been engineered to overexpress an exogenous/heterologous enzyme.

Thus, the DNA-molecules, vectors and host cells employed in connection with the present invention are preferably non-naturally occurring DNA-molecules, vectors and host cells, respectively, i.e. they are DNA-molecules, vectors and host cells which differ significantly from naturally occurring DNA-molecules, vectors and host cells and which do not occur in nature.

Also variants of the host cells are included. Such variants include preferably genetically modified organisms as described herein above which differ from naturally occurring organisms due to a genetic modification. Genetically modified organisms are organisms which do not naturally occur, i.e., which cannot be found in nature, and which differ substantially from naturally occurring organisms due to the introduction of a foreign (recombinant) nucleic acid molecule. Such organisms are also referred to as recombinant.

In a further embodiment the invention relates to the use of the DNA-molecule according to the present invention for regulating expression from a DNA-molecule. It will be understood by those of ordinary skills in the art that in this embodiment the DNA-molecule in addition to the sequence encoding the RNA-polymerase binding aptamer according to the present invention further may comprise the sequence to be expressed.

The invention also relates to a nucleic acid hybridizing to an RNA-molecule or a DNA-molecule according to the present invention under stringent conditions. Hybridization conditions are known to those skilled in the art and can be found in Current Protocols in Molecular Biology, John Wiley & Sons, N.Y., 6.3.1-6.3.6, 1991. Stringent conditions are defined as equivalent to hybridization in 6× sodium chloride/sodium citrate (SSC) at 45° C., followed by a wash in 0.2×SSC, 0.1% SDS at 65° C.

The DNA molecule according to the present invention is preferably comprised in a vector. In case it comprises an expression cassette the vector contains said DNA-molecule is also referred to as an expression vector or a vector for expression.

The term “vector” refers to a polynucleotide or a mixture of polynucleotides and polypeptides/proteins which is capable of being introduced or of introducing the nucleic acid comprised into a cell. It is preferred that the sequences to be expressed and encoded by the introduced polynucleotide are expressed within the cell upon introduction of the vector.

In a preferred embodiment the vector of the present invention comprises plasmids, phagemids, phages, cosmids, artificial mammalian chromosomes, knock-out or knock-in constructs, viruses, in particular adenoviruses, vaccinia viruses, attenuated vaccinia viruses, canary pox viruses, lentivirus (Chang, L. J. and Gay, E. E. (20001) Curr. Gene Therap. 1:237-251), herpes viruses, in particular Herpes simplex virus (HSV-1, Carlezon, W. A. et al. (2000) Crit. Rev. Neurobiol.), baculovirus, retrovirus, adeno-associated-virus (AAV, Carter, P. J. and Samulski, R. J. (2000) J. Mol. Med 6:17-27), rhinovirus, human immune deficiency virus (HIV), filovirus and engineered versions thereof (see, for example, Cobinger G. P. et al (2001) Nat. Biotechnol. 19:225-30), virosomes, “naked” DNA liposomes, and nucleic acid coated particles, in particular gold spheres. Particularly preferred are viral vectors like adenoviral vectors or retroviral vectors (Lindemann et al. (1997) Mol. Med 3:466-76 and Springer et al. (1998) Mol. Cell. 2:549-58). Liposomes are usually small unilamellar or multilamellar vesicles made of cationic, neutral and/or anionic lipids, for example, by ultrasound treatment of liposomal suspensions. The DNA can, for example, be ionically bound to the surface of the liposomes or internally enclosed in the liposome. Suitable lipid mixtures are known in the art and comprise, for example, DOTMA (1,2-Dioleyloxpropyl-3-trimethylammoniumbromid) and DPOE (Dioleoylphosphatidyl-ethanolamin) which both have been used on a variety of cell lines. The vector according to the present invention may serve for different purposes as outlined herein. It may for example be used to introduce a DNA molecule comprising a sequence encoding a RNA-polymerase binding aptamer according to the present invention into an existing gene in order to regulate, e.g. enhance/increase or inhibit/repress/decrease, expression of the gene. Hence, vector according to the present invention at least comprises a DNA-molecule comprising a sequence encoding a RNA-polymerase binding aptamer according to the present invention. In a preferred embodiment the vector comprises a DNA-molecule, said DNA-molecule comprising an expression cassette according to the present invention.

Furthermore, the DNA-molecule of the vector of the present invention may, in addition to the sequence encoding the RNA-polymerase binding aptamer of the invention, comprise further expression control elements, allowing proper expression of the coding regions in suitable hosts. Such control elements are known to the skilled person and may include a promoter, a splice cassette, translation initiation codon, translation and insertion site for introducing an insert into the vector (also referred to as multiple cloning site). Preferably, the sequence encoding the RNA-polymerase binding aptamer of the invention is operatively linked to said further expression control sequences allowing expression of a sequence to be expressed and to be inserted into said multiple cloning site in eukaryotic or prokaryotic cells. Accordingly, the present invention relates to a vector comprising DNA molecule comprising a sequence encoding the RNA-polymerase binding aptamer of the invention, wherein the nucleic acid is operably linked to a promoter that is recognized by a host cell when the eukaryotic and/or prokaryotic (host) cell is transfected with the vector. In a preferred embodiment the vector preferably comprises a ribosomal binding site, wherein said ribosomal binding site is located downstream of said sequence encoding the RNA-polymerase binding aptamer.

Furthermore, the vector of the present invention is preferably an expression vector, i.e. comprising a DNA-molecule, said DNA-molecule comprising an expression cassette according to the present invention. The DNA-molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system of the invention. An expression vector, otherwise known as an expression construct, is usually a plasmid or virus designed for protein expression in cells. The vector is used to introduce a specific gene into a target cell, and can commandeer the cell's mechanism for protein synthesis to produce the protein encoded by the gene. The plasmid is engineered to contain regulatory sequences that act as enhancer and/or promoter regions and lead to efficient transcription of the gene carried on the expression vector. The aim of an expression vector is the production of significant amount of stable messenger RNA, and consequently proteins or polypeptides. An expression vector has features that any vector may have, such as an origin of replication, a selectable marker, and a suitable site for the insertion of a gene such as the multiple cloning site. The cloned gene may be transferred from a specialized cloning vector to an expression vector, although it is possible to clone directly into an expression vector. The cloning process is normally performed in bacteria, e.g. Escherichia coli, and vectors used for protein expression in organisms other than the one used for cloning, e.g. E. coli, may have, in addition to a suitable origin of replication for its propagation in the cloning organism, elements that allow them to be maintained in another organism, and these vectors are called shuttle vectors. An expression vector has elements necessary for protein expression. These may include a promoter, constitutive or inducible, the correct translation initiation sequence such as a ribosomal binding site and/or start codon, a termination codon, and a transcription termination sequence. There are differences in the machinery for protein synthesis between prokaryotes and eukaryotes, therefore the expression vectors must have the elements for expression that is appropriate for the chosen host. For example, prokaryotes expression vectors may have a Shine-Dalgarno sequence at its translation initiation site for the binding of ribosomes, while eukaryotes expression vectors would contain the Kozak consensus sequence. The vector according to the present invention is preferably a vector is for expressing a sequence of interest in bacteria, preferably in Escherichia coli.

The promoter initiates the transcription and is therefore a point of control for the expression of the cloned gene. The promoters used in expression vector are constitutive or inducible. The latter promoters meaning that protein synthesis is only initiated or fully initiated, when required, e.g. by the introduction of an inducer, such as IPTG or arabinose. Protein expression however may also be constitutive (i.e. transcription is constant and the protein is constantly expressed) in some expression vectors. Low level of constitutive protein synthesis may occur even in expression vectors with tightly controlled promoters.

After the expression of the gene product, it is usually necessary to purify the expressed protein; however, separating the protein of interest from the great majority of proteins of the host cell can be a protracted process. To make this purification process easier, a purification tag may be added to the cloned gene or being present in an expression cassette or vector to allow cloning of a sequence of interest such that the gene and the tag are in frame and expressed as a single polypeptide chain. Such tags optionally being present in the vector according to the present invention include histidine (His) tag, marker peptides, and fusion partners, such as glutathione S-transferase or maltose-binding protein or GFP or β-galactosidase. Some of these fusion partners may also help to increase the solubility of some expressed proteins. Other fusion proteins such as green fluorescent protein may act as a reporter gene for the identification of successful cloned genes.

The vector or DNA-molecule comprising an expression cassette may be transformed or transfected (also referred to herein as introduced) into the host cell for protein synthesis. The vectors of the invention may have element for transformation or the insertion of DNA into the host chromosome, for example the vir genes for plant transformation, and integrase sites for chromosomal insertion. The vector of the invention may include targeting sequence that may target the expressed protein to a specific location such as the periplasmic space of bacteria.

The vector may include further control elements ensuring and/or regulating expression. Control elements ensuring expression in eukaryotic and prokaryotic (host) cells are well known to those skilled in the art. As mentioned herein above, they usually comprise regulatory sequences ensuring initiation of transcription and optionally signals ensuring termination of transcription and stabilization of the transcript. Additional regulatory elements may include transcriptional as well as translational enhancers, and/or naturally-associated or heterologous promoter regions.

The promoter according to the invention is preferably constitutive or inducible as outlined herein. Preferred promoters according to the present invention include prokaryotic and eukaryotic promoters, such as bacterial, fungal or mammalian promoters. Preferred mammalian promoters are selected from the group consisting of CMV-HSV thymidine kinase promoter, SV40, RSV-promoter (Rous Sarcoma Virus), human elongation factor 1α-promoter, the glucocorticoid-inducible MMTV-promoter (Moloney Mouse Tumor Virus), neurofilament-promoter, PGDF-promoter, NSE-promoter, PrP-promoter, thy-1-promoter, metallothionein-inducible promoter, and tetracyclin-inducible promoter. Furthermore, enhancers may be present in the vector or DNA-molecule according to the present invention. Preferred enhancers are selected from the group consisting of CMV enhancer, and SV40-enhancer. For expression in neural cells, it is envisaged that neurofilament-, PGDF-, NSE-, PrP-, or thy-1-promoters can be employed. Said promoters are known in the art and, inter alia, described in Charron J. Biol. Chem. 270 (1995), 25739-25745. Preferred promoters are outlined herein above for the DNA-molecule according to the present invention and apply also to the vectors and plasmids.

Besides elements which are responsible for the initiation of transcription such regulatory elements may also comprise transcription termination signals, such as SV40-poly-A site or the tk-poly-A site, downstream of the sequence to be expressed. In this context, suitable expression vectors are known in the art such as Okayama-Berg cDNA expression vector pcDV1 (Pharmacia), pRc/CMV, pcDNA1, pcDNA3 (In-vitrogene), pSPORT1 (GIBCO BRL), pX (Pagano, Science 255 (1992), 1144-1147), yeast two-hybrid vectors, such as pEG202 and dpJG4-5 (Gyuris, Cell 75 (1995), 791-803), or preferred prokaryotic expression vectors, such as pET21a (Novagen), pWM015 (W. G. Miller et al., Appl. Environ. Microbiol. 66, 5426-5436 (2000)), pBAD24 (L. M. Guzman et al., 177 (1995), Lambda gt11 or pGEX (Amersham-Pharmacia). The vector may further comprise nucleic acid sequences encoding for a secretion signal. Such sequences are well known to the person skilled in the art. Furthermore, depending on the expression system used, leader sequences capable of directing the peptides of the invention to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein, or a protein thereof, into the periplasmic space or extracellular medium. Optionally, the heterologous sequence can encode a fusion protein including a C- or N-terminal identification peptide imparting desired characteristics, e.g., stabilization or simplified purification of expressed recombinant products. Once the vector has been incorporated into the appropriate host, the host is maintained under conditions suitable maintaining the vector and optionally expression of the sequence to be expressed, and, as desired, the collection and purification of the so expressed molecules may follow.

The present invention also relates to a host cell transfected or transformed, i.e. comprising with the vector or DNA-molecule of the invention or a non-human host organism carrying the vector of the present invention, i.e. to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

The skilled person will acknowledge that the DNA-molecules according to the present invention are also applicable for gene therapy in a subject. Hence, the present invention also relates to the DNA-molecule according to the present invention for use in gene therapy in a subject. “Subject” in the meaning of the invention is understood to be all persons, animals, plants or microorganisms, irrespective whether or not they exhibit pathological changes, unless stated otherwise. In a preferred embodiment the patient according to the invention is a human. In a further preferred embodiment of the invention the patient is a human suspected to have a disease to be treated by gene therapy.

For medical use the DNA-molecule or vector or plasmid may be presented in as a pharmaceutical composition. The pharmaceutical composition may comprise further pharmaceutical excipients deemed necessary, e.g. for stabilizing the DNA-molecule, for diminishing adverse effects of the subject upon application of the composition etc. The DNA-molecule or vector or plasmid may be present in pure form or as a pharmaceutically acceptable salt thereof. The phrase “pharmaceutically acceptable salt(s)”, as used herein, denotes those salts of compounds of the invention that are nontoxic, safe and effective for medical use in mammals and that possess the desired biological activity, including, but not limited to, acid addition and/or base salts. The acid addition salts are formed from basic invention compounds, whereas the base addition salts are formed from acidic invention compounds. All of these forms are within the scope of the compounds useful in the invention. For a review on pharmaceutically acceptable salts see Berge et al. (1977), J. Pharm. Sci. 66:1-19, incorporated herein by reference.

The present invention furthermore relates to the use of a DNA-molecule comprising sequence encoding a RNA-polymerase binding aptamer according to the present invention for regulating expression of a protein, preferably for regulating expression of a protein in a microorganism, such as prokaryotic or eukaryotic cell lines, including bacteria and mammal cell lines. The protein to be expressed is preferably encoded on the same DNA-molecule as the RNA-polymerase binding aptamer, downstream of the RNA-polymerase binding aptamer sequence and preferably positioned such that it is transcribed into the same RNA molecule. In a preferred embodiment of the present invention said sequence encoding said RNA-polymerase binding aptamer is positioned downstream of the promoter from which the expression of said protein is initiated. This means that the RNA-polymerase binding aptamer is part of the mRNA encoding the protein to be expressed. The use according to the present invention encompasses the use for regulating the expression of any protein of interest. A protein of interest may be any protein that can be expressed in expression systems. Such expression systems usually employs host cells transfected or transformed with a DNA-molecule, e.g. a vector, according to the present invention. Hence, the invention also encompasses the use of a host cell according to the present invention for expression of a sequence of interest, e.g. a protein.

The present invention relates to methods of producing a protein of interest comprising providing a vector according to the present invention, preferably comprising an expression cassette according to the present invention, said vector comprising a sequence encoding the protein of interest operatively linked to the promoter and the sequence encoding the RNA-polymerase binding aptamer, introducing said vector into a host cell, and culturing said host cell in culture medium under conditions inducing transcription from the promoter of the expression vector, and optionally recovering the protein of interest from the host cell or culture medium.

It will be understood by the skilled artisan that the provision of the vector comprising the expression cassette may include the cloning of the sequence to be expressed into an expression cassette according to the present invention. Hence, in one embodiment the step of providing a vector according to the present invention may include cloning of the sequence encoding said protein of interest into said vector such that the sequence is operatively linked to the promoter and the sequence encoding the RNA-polymerase binding aptamer to obtain the final expression vector, preferably cloning into a multiple cloning site of the expression cassette. Hence, the invention also relates to a method methods of producing a protein of interest comprising the steps of providing a vector according to the present invention, preferably comprising an expression cassette according to the present invention, cloning the sequence encoding a protein of interest into said vector, preferably into a multiple cloning site, such that the sequence is operatively linked to the promoter and the sequence encoding the RNA-polymerase binding aptamer to obtain the final expression vector, introducing said expression vector into a host cell, and culturing said host cell in culture medium under conditions inducing transcription from the promoter of the expression vector, and optionally recovering the protein of interest from the host cell or culture medium.

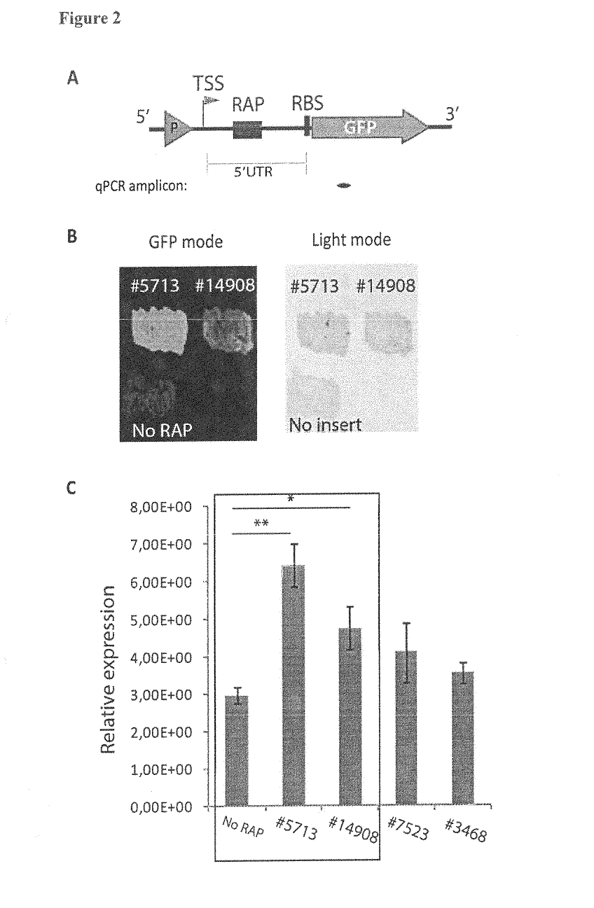

It has been found by the inventors that a subgroup of the RNA-polymerase binding aptamers according to the present invention increase the activity of RNA polymerase when transcribed from a DNA strand encoding said aptamer. The efficiency of transcription of a DNA segment is increased if the DNA segment comprises a sequence encoding a RNA-polymerase binding aptamer according to the invention. Hence, in one preferred embodiment of the present invention the RNA-polymerase binding aptamer increases the activity of the RNA-polymerase. The skilled person is aware of methods, assays and systems to determine activity of RNA-polymerases. Such methods include the expression of reporter genes in a host cell or may apply in vitro transcription assays as both outlined in detail in the enclosed Examples. In host cell based assays basically a DNA construct is used which comprises a promoter to allow start of transcription and a reporter, e.g. a reporter gene. To test for the increase of RNA-polymerase activity the expression of the construct starting from the promoter is measured and compared between a construct comprising a sequence encoding an RNA-polymerase binding aptamer according to the present invention and the construct not comprising said aptamer. Expression can be measured using methods commonly known by the skilled artisan. For example, the activity of a protein encoded by the reporter gene can be tested, e.g. fluorescence or enzymatic activity. Additionally or alternatively, mRNA amounts as the direct transcription product can be determined using common techniques, like Northern Blot or quantitative reverse transcription (qRT) real-time PCR. For example, to investigate the effect of a sequence encoding a RNA-polymerase binding aptamer on RNA-polymerase activity and thereby on transcription a plasmid-based GFP reporter system may be used. Possible systems are used in the presented Examples (see e.g. FIG. 2A). However, as will be understood by the skilled artisan the invention is not restricted to a certain reporter. For example also other fluorescent proteins may be used, like RFP, YFP, etc. Also enzyme based systems are applicable. A further method to investigate impact of a sequence on transcription makes use of β-galactosidase as the encoded product of the reporter gene lacZ, as also outlined in the Examples of the present invention. In the assay to determine the effect of the RNA-polymerase binding aptamer, the DNA sequence encoding said aptamer is preferably located downstream of the promoter, more preferably downstream of the transcription start site. It is further preferred that the DNA sequence encoding said aptamer is located upstream of the reporter.

The inventors further found that some of the RNA-polymerase binding aptamer increasing the activity of the RNA-polymerase are increasing the activity of the polymerase itself (also referred to herein as “activating RNA-polymerase binding aptamers”) and others or the same additionally increase the expression in a terminator-dependent fashion, i.e. the suppress the terminating effect of terminator sequences (also referred to herein as “antiterminating RNA-polymerase binding aptamers”). Both, activating and antiterminating, types of RNA-polymerase binding aptamers, are referred to as “transcription enhancing aptamer”.

Two classes of transcription terminators in prokaryotes, Rho-dependent and Rho-independent, have been identified throughout prokaryotic genomes. These widely distributed sequences are responsible for triggering the end of transcription upon normal completion of gene or operon transcription, mediating early termination of transcripts as a means of regulation such as that observed in transcriptional attenuation, and to ensure the termination of runaway transcriptional complexes that manage to escape earlier terminators by chance, which prevents unnecessary energy expenditure for the cell. Rho-dependent transcription terminators require a protein called Rho factor, which exhibits RNA helicase activity, to disrupt the mRNA-DNA-RNA polymerase transcriptional complex. Rho-dependent terminators are found in bacteria and phages. The Rho-dependent terminator occurs downstream of translational stop codons and consists of an unstructured, cytosine-rich sequence on the mRNA known as a Rho utilization site (rut), and a downstream transcription stop point (tsp). The rut serves as an mRNA loading site and as an activator for Rho; activation enables Rho to efficiently hydrolyze ATP and translocate down the mRNA while it maintains contact with the rut site. Rho is able to catch up with the RNA polymerase, which is stalled at the downstream tsp sites (see e.g. Richardson, J. P. (1996). “Rho-dependent Termination of Transcription Is Governed Primarily by the Upstream Rho Utilization (rut) Sequences of a Terminator”. Journal of Biological Chemistry 271 (35): 21597-21603). Contact between Rho and the RNA polymerase complex stimulates dissociation of the transcriptional complex through a mechanism involving allosteric effects of Rho on RNA polymerase (see e.g. Ciampi, M S. (September 2006). “Rho-dependent terminators and transcription termination”, Microbiology, 152(Pt 9): 2515-2528; and Epshtein, V; Dutta, D; Wade, J; Nudler, E (Jan. 14, 2010). “An allosteric mechanism of Rho-dependent transcription termination”, Nature, 463 (7278): 245-249).

Intrinsic transcription terminators or Factor/Rho-independent terminators require the formation of a self-annealing hairpin structure on the elongating transcript, which results in the disruption of the mRNA-DNA-RNA polymerase ternary complex. The terminator sequence contains a 20 basepair GC-rich region of dyad symmetry followed by a short poly-T tract or “T stretch” which is transcribed to RNA to form the terminating hairpin and a 7-9 nucleotide “U track” respectively. The mechanism of termination is hypothesized to occur through a combination of direct promotion of dissociation through allosteric effects of hairpin binding interactions with the RNA polymerase and “competitive kinetics”. The hairpin formation causes RNA polymerase stalling and destabilization, leading to a greater likelihood that dissociation of the complex will occur at that location due to an increased time spent paused at that site and reduced stability of the complex (see e.g. von Hippel, P. H. (1998). “An Integrated Model of the Transcription Complex in Elongation, Termination, and Editing” Science 281 (5377): 660-665; and Gusarov, Ivan; Nudler, Evgeny (1999). “The Mechanism of Intrinsic Transcription Termination” Molecular Cell 3 (4): 495-504).

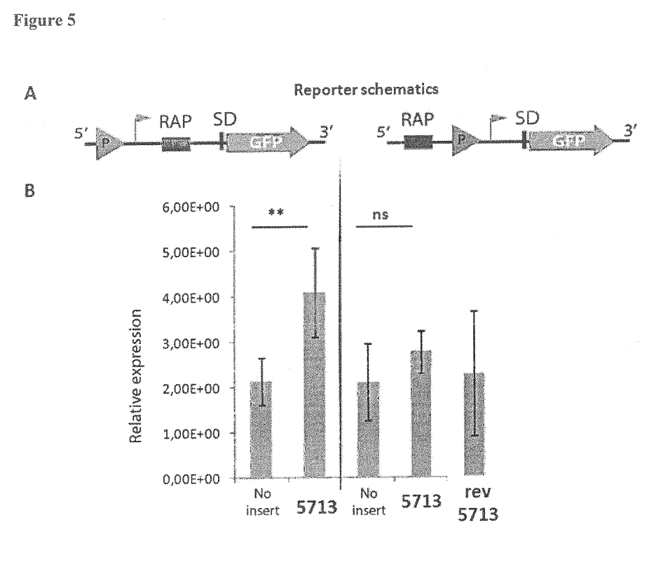

The antiterminating RNA-polymerase binding aptamers suppress termination as shown in the examples. Such suppression may be a suppression of termination through intrinsic terminators (factor independent) and factor-dependent terminators. Two types have been for example described in Santangelo and Artsimovitch (2011), Nat. Rev. Microbiol. 9:319-329, which is incorporated herein by reference. The skilled person when considering the disclosure of the present invention and his common knowledge is in the position to test the ability of a certain aptamer for suppressing termination of transcription. The skilled person is aware of methods, assays and systems to determine activity of RNA-polymerases, supra. Such methods include the expression of reporter genes in a host cell or may apply in vitro transcription assays as outlined in detail in the enclosed Examples. In host cell based assays basically a DNA construct is used which comprises a promoter to allow start of transcription and a reporter, e.g. a reporter gene. To test for the increase of RNA-polymerase activity the expression of the construct starting from the promoter is measured and compared between a construct comprising a sequence encoding an RNA-polymerase binding aptamer according to the present invention and the construct not comprising said aptamer. When testing for the suppression of termination, the construct shall further comprise, in both constructs, the control and the aptamer containing construct, the terminator sequence, e.g. rrnB or T7t terminators (see e.g. Santangelo and Artsimovitch (2011), Nat. Rev. Microbiol. 9:319-329). The terminator is preferably located downstream of the sequence encoding the RNA-polymerase binding aptamer. Expression can be measured using methods commonly known by the skilled artisan, supra. For example, the activity of a protein encoded by the reporter gene can be tested, e.g. fluorescence or enzymatic activity. Additionally or alternatively, mRNA amounts as the direct transcription product can be determined using common techniques, like Northern Blot or quantitative reverse transcription (qRT) real-time PCR. For example, to investigate the effect of a sequence encoding a RNA-polymerase binding aptamer on RNA-polymerase activity and thereby on transcription a plasmid-based GFP reporter system may be used. Possible systems are used in the presented Examples (see e.g. FIG. 5A). However, as will be understood by the skilled artisan the invention is not restricted to a certain reporter. For example also other fluorescent proteins may be used, like RFP, YFP, etc. Also enzyme based systems are applicable. A further method to investigate impact of a sequence on transcription makes use of β-galactosidase as the encoded product of the reporter gene lacZ, as also outlined in the Examples of the present invention. In the assay to determine the effect of the RNA-polymerase binding aptamer, the DNA sequence encoding said aptamer is preferably located downstream of the promoter, more preferably downstream of the transcription start site, and preferably upstream of the terminator. It is further preferred that the DNA sequence encoding said aptamer is located upstream of the reporter.

Even though activating and antiterminating mechanisms could be identified, several identified aptamers have both activities, i.e. enhancing RNA-polymerase transcription activity in a terminator dependent as well as a terminator independent way. Examples of such enhancing aptamers acting through both mechanisms are the RNA-polymerase binding aptamers encoded by SEQ ID NOs:2 to 24, which are particularly preferred herein.

However, in one embodiment of the present invention the enhancing RNA-polymerase binding aptamer according to the invention is an anti-terminating RNA-polymerase binding aptamers and enhances the activity of the RNA-polymerase in a terminator-dependent fashion. In a further embodiment the enhancing RNA-polymerase binding aptamer according to the invention is an activating RNA-polymerase binding aptamers and enhances the activity of the RNA-polymerase in a terminator-independent fashion. In yet a particular preferred embodiment, the enhancing RNA-polymerase binding aptamer according to the invention is an activating and antiterminating RNA-polymerase binding aptamers and enhances the activity of the RNA-polymerase in a terminator-dependent and terminator-independent fashion.

Transcription terminators are well known by the skilled person and include factor-dependent as well as intrinsic terminator sequences. Examples of prokaryotic terminators are for example disclosed in Santangelo and Artsimovitch (2011), Nat. Rev. Microbiol. 9:319-329. In a preferred embodiment of the present invention the term “terminator” refers to a terminator selected from rut (Rho dependent terminator), rrnB, 77t and tR2 terminators (intrinsic terminator sequences). Preferred terminator sequences are shown in Table 3 and have a sequence selected from the group consisting of SEQ ID NO:42, SEQ ID NO:43, and SEQ ID NO:44.

In a preferred embodiment of the present invention the RNA-polymerase binding aptamer increases expression of a sequence to be expressed (also referred to as “transcription enhancing aptamer” or “transcription enhancing RNA-polymerase binding aptamer”), wherein the increase of expression is at least 10% as compared to the expression of said sequence to be expressed when not comprising said RNA-polymerase binding aptamer, preferably by at least 20%, more preferably at least 100%. Preferably the RNA-polymerase binding aptamer increases expression of a sequence to be expressed in a reporter assay as outlined herein.

Antiterminating activity of a RNA-polymerase binding aptamer can be tested by the skilled person when considering the disclosure of the present application. In a preferred embodiment of the present invention the antiterminating RNA-polymerase binding aptamer increases expression of a sequence to be expressed comprising a terminator sequence, wherein the increase of expression is at least 10% as compared to the expression of said sequence to be expressed when not comprising said antiterminating RNA-polymerase binding aptamer, preferably by at least 20%, more preferably at least 100%. Preferably the antiterminating RNA-polymerase binding aptamer increases expression of a sequence to be expressed comprising a terminator sequence in a reporter assay as outlined herein.

The inventors identified a plurality of RNA-polymerase binding aptamers (SEQ ID NOs:2 to 24) that possess the feature of increasing the expression of a sequence to be expressed, these are preferably of bacterial origin. Detailed analysis of the sequences revealed a common motif comprised in these sequences. This RNA sequence is encoded by the DNA sequence of G[A/G][C/A/T][A/T][A/G/T][C/G]AT[G/T/C][A/C]G[G/A][G/T][C/A][A/G][G/A/C][T/G][T/A/G][C/T/G]AGCA and outlined herein as SEQ ID NO:1 using the expanded one letter code (see Table 2). Hence, in one embodiment of the RNA-polymerase binding aptamer, the RNA-polymerase binding aptamer is an transcription enhancing aptamer and encoded by a sequence of SEQ ID NO:1. In a further preferred embodiment the RNA-polymerase binding aptamer is an activating RNA-polymerase binding aptamer and comprises a sequence encoded by a sequence of SEQ ID NO:1. In yet a further embodiment the RNA-polymerase binding aptamer is an antiterminating RNA-polymerase binding aptamer and comprises a sequence encoded by a sequence of SEQ ID NO:1. It has been found that this consensus sequence is present in DNAs encoding transcription enhancing RNA-polymerase binding aptamers derived of bacterial origin, e.g. E. coli. A further consensus sequence has been identified in DNA encoding for transcription enhancing RNA-polymerase binding aptamers derived from eukaryotic origin, i.e. SEQ ID NO:138. The thereby encoded transcription enhancing RNA-polymerase binding aptamers have also been shown to haven transcription enhancing properties in bacterial expression systems, such as E. coli; see Examples. Further, it has been found using GLAM2 that the enhancing sequences, i.e. the sequences sharing motif of SEQ ID NO:1 or SEQ ID NO:138, all share the common motif of SEQ ID NO:139. This motif furthermore fits the identified essential nt as tested for the aptamer encoded by SEQ ID NO:2; see Examples.

Hence, in a preferred embodiment of the present invention the transcription enhancing RNA-polymerase binding aptamer is encoded by a sequence according to SEQ ID NO:139 (CAN₀₋₃[AC][CA]N[GC][AT]N[CA][CA][CAT]). A sequence according to SEQ ID NO:139 is preferably selected from the group consisting of SEQ ID NO:1 and SEQ ID NO:138. Particular preferred sequences of transcription enhancing RNA-polymerase binding aptamers are encoded by a sequence according to SEQ ID NO:1. Preferred sequences falling within the consensus of SEQ ID NO:1 are selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75, or a sequence at least 80% identical to any of these sequences, more preferably SEQ ID NO:2 or a sequence at least 80% identical thereto. Preferred sequences falling within the consensus of SEQ ID NO:138 are selected from the group consisting of SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137 or a sequence at least 80% identical to any of these sequences.

The inventors found a plurality of RNA-polymerase binding aptamers which enhance the activity of the RNA-polymerase they are binding to. Furthermore, a common motif has been identified as SEQ ID NO:1. Furthermore, the inventors demonstrated that variations within the sequence of one example aptamer are possible at certain positions without interfering with its properties. Hence the skilled person will instantly acknowledge that the aptamers of certain sequences as disclosed herein retain their properties. Hence, the transcription enhancing RNA-polymerase binding aptamer according to the present invention is in one embodiment encoded by a sequence having at least 80% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75 SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75. In a further embodiment the transcription enhancing RNA-polymerase binding aptamer according to the present invention is encoded by a sequence having at least 90% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75. In yet a further embodiment the transcription enhancing RNA-polymerase binding aptamer according to the present invention is encoded by a sequence having at least 95% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75 SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75. In a preferred embodiment the transcription enhancing RNA-polymerase binding aptamer according to the present invention is encoded by a sequence having at least 99% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75. In a particular embodiment the transcription enhancing RNA-polymerase binding aptamer according to the present invention is encoded by a sequence having a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137; preferably selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75. A particular preferred transcription enhancing RNA-polymerase binding aptamer according to the present invention is encoded by the sequence of SEQ ID NO:2 or a sequence having at least 80% identity thereto, preferably at least 90% identity, more preferably at least 95% identity, yet more preferred at least 97% identity, even more preferred at least 99% identity. The invention also relates to a nucleic acid hybridizing to any of these sequences under stringent conditions.

In a preferred embodiment of the present invention the transcription enhancing RNA-polymerase binding aptamer according to the present invention is encoded by a sequence having at least 80% identity to SEQ ID NO:2, preferably by a sequence having at least 90% identity, more preferably by a sequence having at least 95% identity, even more preferred by a sequence having at least 99% identity to SEQ ID NO:2. In a particular embodiment the RNA-polymerase binding aptamer is encoded by the sequence of SEQ ID NO:2. In a preferred embodiment of the transcription enhancing RNA-polymerase binding aptamer being encoded by a sequence having a certain identity to SEQ ID NO:2, the sequence encoding the aptamer is not altered at the nucleotides at positions 1, 7, 8, 11, and 20 to 23 of SEQ ID NO:2. The inventors have shown that the sequences encoding the aptamer may be mutated without loosing its activity. In particular, it has been shown that variants of RAP ID #5713 as encoded by SEQ ID NO:2, i.e. SEQ ID NOs: 72 to 75 retain their property as transcription enhancing RNA-polymerase binding aptamers. Hence, in one embodiment the (transcription enhancing) RNA-polymerase binding aptamer according to the present invention is encoded by a sequence selected from the group consisting of SEQ ID NO:2, SEQ ID NO:72, SEQ ID NO:73, SEQ ID NO:74, and SEQ ID NO:75. The sequence of SEQ ID NO:75 is the sequence of SEQ ID NO:2 shortened at its 3′-end by 6 nt and thereby only comprising the part of SEQ ID NO:2 corresponding to the minimal consensus sequence of SEQ ID NO:1. The thereby encoded aptamer retains its function. Hence, in a particular preferred embodiment the (transcription enhancing) RNA-polymerase binding aptamer according to the present invention is encoded by a sequence comprising a sequence having at least 80% identity to SEQ ID NO:75, preferably comprising a sequence having at least 90% identity, more preferably comprising a sequence having at least 95% identity, even more preferred comprising a sequence having at least 99% identity to SEQ ID NO:75. In a particular embodiment the transcription enhancing RNA-polymerase binding aptamer is encoded by a sequence comprising the sequence of SEQ ID NO:75. In yet a further particular preferred embodiment the transcription enhancing RNA-polymerase binding aptamer is encoded by a sequence having the sequence of SEQ ID NO:75. In a preferred embodiment of the transcription enhancing RNA-polymerase binding aptamer being encoded comprising or consisting of a sequence having a certain identity to SEQ ID NO:75, the sequence encoding the aptamer is not altered at the nucleotides at positions 1, 7, 8, 11, and 20 to 23 of SEQ ID NO:75.

In a preferred embodiment of the present invention the transcription enhancing RNA-polymerase binding aptamer according to the present invention is encoded by a sequence having at least 80% identity to SEQ ID NO:3, preferably by a sequence having at least 90% identity, more preferably by a sequence having at least 95% identity, even more preferred by a sequence having at least 99% identity to SEQ ID NO:3. In a particular embodiment the RNA-polymerase binding aptamer is encoded by the sequence of SEQ ID NO:3.

In a preferred embodiment of the present invention the transcription enhancing RNA-polymerase binding aptamer according to the present invention is encoded by a sequence having at least 80% identity to SEQ ID NO:4, preferably by a sequence having at least 90% identity, more preferably by a sequence having at least 95% identity, even more preferred by a sequence having at least 99% identity to SEQ ID NO:4. In a particular embodiment the RNA-polymerase binding aptamer is encoded by the sequence of SEQ ID NO:4.

In a preferred embodiment of the present invention the transcription enhancing RNA-polymerase binding aptamer according to the present invention is encoded by a sequence having at least 80% identity to SEQ ID NO:5, preferably by a sequence having at least 90% identity, more preferably by a sequence having at least 95% identity, even more preferred by a sequence having at least 99% identity to SEQ ID NO:5. In a particular embodiment the RNA-polymerase binding aptamer is encoded by the sequence of SEQ ID NO:5.

In a preferred embodiment of the present invention the transcription enhancing RNA-polymerase binding aptamer according to the present invention is encoded by a sequence having at least 80% identity to SEQ ID NO:6, preferably by a sequence having at least 90% identity, more preferably by a sequence having at least 95% identity, even more preferred by a sequence having at least 99% identity to SEQ ID NO:6. In a particular embodiment the RNA-polymerase binding aptamer is encoded by the sequence of SEQ ID NO:6.

In a preferred embodiment of the present invention the transcription enhancing RNA-polymerase binding aptamer according to the present invention is encoded by a sequence having at least 80% identity to SEQ ID NO:7, preferably by a sequence having at least 90% identity, more preferably by a sequence having at least 95% identity, even more preferred by a sequence having at least 99% identity to SEQ ID NO:7. In a particular embodiment the RNA-polymerase binding aptamer is encoded by the sequence of SEQ ID NO:7.

In a preferred embodiment of the present invention the transcription enhancing RNA-polymerase binding aptamer according to the present invention is encoded by a sequence having at least 80% identity to SEQ ID NO:8, preferably by a sequence having at least 90% identity, more preferably by a sequence having at least 95% identity, even more preferred by a sequence having at least 99% identity to SEQ ID NO:8. In a particular embodiment the RNA-polymerase binding aptamer is encoded by the sequence of SEQ ID NO:8.

In a preferred embodiment of the present invention the transcription enhancing RNA-polymerase binding aptamer according to the present invention is encoded by a sequence having at least 80% identity to SEQ ID NO:9, preferably by a sequence having at least 90% identity, more preferably by a sequence having at least 95% identity, even more preferred by a sequence having at least 99% identity to SEQ ID NO:9. In a particular embodiment the RNA-polymerase binding aptamer is encoded by the sequence of SEQ ID NO:9.

In a preferred embodiment of the present invention the transcription enhancing RNA-polymerase binding aptamer according to the present invention is encoded by a sequence having at least 80% identity to SEQ ID NO:10, preferably by a sequence having at least 90% identity, more preferably by a sequence having at least 95% identity, even more preferred by a sequence having at least 99% identity to SEQ ID NO:10. In a particular embodiment the RNA-polymerase binding aptamer is encoded by the sequence of SEQ ID NO:10.

In a preferred embodiment of the present invention the transcription enhancing RNA-polymerase binding aptamer according to the present invention is encoded by a sequence having at least 80% identity to SEQ ID NO:11, preferably by a sequence having at least 90% identity, more preferably by a sequence having at least 95% identity, even more preferred by a sequence having at least 99% identity to SEQ ID NO:11. In a particular embodiment the RNA-polymerase binding aptamer is encoded by the sequence of SEQ ID NO:11.

In a preferred embodiment of the present invention the transcription enhancing RNA-polymerase binding aptamer according to the present invention is encoded by a sequence having at least 80% identity to SEQ ID NO:12, preferably by a sequence having at least 90% identity, more preferably by a sequence having at least 95% identity, even more preferred by a sequence having at least 99% identity to SEQ ID NO:12. In a particular embodiment the RNA-polymerase binding aptamer is encoded by the sequence of SEQ ID NO:12.

In a preferred embodiment of the present invention the transcription enhancing RNA-polymerase binding aptamer according to the present invention is encoded by a sequence having at least 80% identity to SEQ ID NO:13, preferably by a sequence having at least 90% identity, more preferably by a sequence having at least 95% identity, even more preferred by a sequence having at least 99% identity to SEQ ID NO:13. In a particular embodiment the RNA-polymerase binding aptamer is encoded by the sequence of SEQ ID NO:13.

In a preferred embodiment of the present invention the transcription enhancing RNA-polymerase binding aptamer according to the present invention is encoded by a sequence having at least 80% identity to SEQ ID NO:14, preferably by a sequence having at least 90% identity, more preferably by a sequence having at least 95% identity, even more preferred by a sequence having at least 99% identity to SEQ ID NO:14. In a particular embodiment the RNA-polymerase binding aptamer is encoded by the sequence of SEQ ID NO:14.

In a preferred embodiment of the present invention the transcription enhancing RNA-polymerase binding aptamer according to the present invention is encoded by a sequence having at least 80% identity to SEQ ID NO:15, preferably by a sequence having at least 90% identity, more preferably by a sequence having at least 95% identity, even more preferred by a sequence having at least 99% identity to SEQ ID NO:15. In a particular embodiment the RNA-polymerase binding aptamer is encoded by the sequence of SEQ ID NO:15.

In a preferred embodiment of the present invention the transcription enhancing RNA-polymerase binding aptamer according to the present invention is encoded by a sequence having at least 80% identity to SEQ ID NO:16, preferably by a sequence having at least 90% identity, more preferably by a sequence having at least 95% identity, even more preferred by a sequence having at least 99% identity to SEQ ID NO:16. In a particular embodiment the RNA-polymerase binding aptamer is encoded by the sequence of SEQ ID NO:16.

In a preferred embodiment of the present invention the transcription enhancing RNA-polymerase binding aptamer according to the present invention is encoded by a sequence having at least 80% identity to SEQ ID NO:17, preferably by a sequence having at least 90% identity, more preferably by a sequence having at least 95% identity, even more preferred by a sequence having at least 99% identity to SEQ ID NO:17. In a particular embodiment the RNA-polymerase binding aptamer is encoded by the sequence of SEQ ID NO:17.

In a preferred embodiment of the present invention the transcription enhancing RNA-polymerase binding aptamer according to the present invention is encoded by a sequence having at least 80% identity to SEQ ID NO:18, preferably by a sequence having at least 90% identity, more preferably by a sequence having at least 95% identity, even more preferred by a sequence having at least 99% identity to SEQ ID NO:18. In a particular embodiment the RNA-polymerase binding aptamer is encoded by the sequence of SEQ ID NO:18.

In a preferred embodiment of the present invention the transcription enhancing RNA-polymerase binding aptamer according to the present invention is encoded by a sequence having at least 80% identity to SEQ ID NO:19, preferably by a sequence having at least 90% identity, more preferably by a sequence having at least 95% identity, even more preferred by a sequence having at least 99% identity to SEQ ID NO:19. In a particular embodiment the RNA-polymerase binding aptamer is encoded by the sequence of SEQ ID NO:19.

In a preferred embodiment of the present invention the transcription enhancing RNA-polymerase binding aptamer according to the present invention is encoded by a sequence having at least 80% identity to SEQ ID NO:20, preferably by a sequence having at least 90% identity, more preferably by a sequence having at least 95% identity, even more preferred by a sequence having at least 99% identity to SEQ ID NO:20. In a particular embodiment the RNA-polymerase binding aptamer is encoded by the sequence of SEQ ID NO:20.

In a preferred embodiment of the present invention the transcription enhancing RNA-polymerase binding aptamer according to the present invention is encoded by a sequence having at least 80% identity to SEQ ID NO:21, preferably by a sequence having at least 90% identity, more preferably by a sequence having at least 95% identity, even more preferred by a sequence having at least 99% identity to SEQ ID NO:21. In a particular embodiment the RNA-polymerase binding aptamer is encoded by the sequence of SEQ ID NO:21.

In a preferred embodiment of the present invention the transcription enhancing RNA-polymerase binding aptamer according to the present invention is encoded by a sequence having at least 80% identity to SEQ ID NO:22, preferably by a sequence having at least 90% identity, more preferably by a sequence having at least 95% identity, even more preferred by a sequence having at least 99% identity to SEQ ID NO:22. In a particular embodiment the RNA-polymerase binding aptamer is encoded by the sequence of SEQ ID NO:22.

In a preferred embodiment of the present invention the transcription enhancing RNA-polymerase binding aptamer according to the present invention is encoded by a sequence having at least 80% identity to SEQ ID NO:23, preferably by a sequence having at least 90% identity, more preferably by a sequence having at least 95% identity, even more preferred by a sequence having at least 99% identity to SEQ ID NO:23. In a particular embodiment the RNA-polymerase binding aptamer is encoded by the sequence of SEQ ID NO:23.

In a preferred embodiment of the present invention the transcription enhancing RNA-polymerase binding aptamer according to the present invention is encoded by a sequence having at least 80% identity to SEQ ID NO:24, preferably by a sequence having at least 90% identity, more preferably by a sequence having at least 95% identity, even more preferred by a sequence having at least 99% identity to SEQ ID NO:24. In a particular embodiment the RNA-polymerase binding aptamer is encoded by the sequence of SEQ ID NO:24.

For the aptamer encoded by the sequences of SEQ ID NOs: 2 to 14, and 72 to 75 antiterminating activity has been observed. Hence, in a preferred embodiment of the present invention the antiterminating RNA-polymerase binding aptamer is encoded by a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:72, SEQ ID NO:73, SEQ ID NO:74, and SEQ ID NO:75, or a sequence having at least 80% identity thereto, preferably at least 90%, more preferably at least 95%, even more preferred at least 99% identity.

The determination of percent identity between two sequences is accomplished using the mathematical algorithm of Karlin and Altschul (1993) Proc. Natl. Acad. Sci. USA 90: 5873-5877. Such an algorithm is incorporated into the BLASTN and BLASTP programs of Altschul et al. (1990) J. Mol. Biol. 215: 403-410. BLAST nucleotide searches are performed with the BLASTN program, score=100, word length=12, to obtain nucleotide sequences homologous to the respective nucleotide sequence. BLAST protein searches are performed with the BLASTP program, score=50, word length=3, to obtain amino acid sequences homologous to the respective amino acid sequence sequence, respectively. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used.

The above outlined embodiments for the DNA-molecule, the expression cassettes and vectors apply to the invention as a whole, i.e. also specifically to the transcription enhancing RNA-polymerase binding aptamers. However, further embodiments in particular apply to the subject-matter relating to said transcription enhancing RNA-polymerase binding aptamers.

As outlined in great detail, the sequence encoding the RNA-polymerase binding aptamer is preferably operatively linked to a promoter and/or a sequence to be expressed. In particularly preferred embodiment of the present invention, the sequence encoding the transcription enhancing RNA-polymerase binding aptamer is operatively linked to a promoter, and optionally to a sequence to be expressed. It has been shown herein, that the transcription enhancing RNA-polymerase binding aptamers are able to increase transcription/expression of a transcript comprising the aptamer. The sequence encoding the transcription enhancing RNA-polymerase binding aptamer according to the present invention is preferably operatively linked to the promoter sequence and the transcription starting site. It is preferably positioned downstream of the promoter and the transcription starting site on the DNA-molecule according to the present invention. The distance of the transcription enhancing RNA-polymerase binding aptamer encoding sequence to the transcription starting site is not crucial. However with certain distances superior effects in terms of regulating the transcription may be observed. Hence, in one embodiment the sequence encoding the transcription enhancing RNA-polymerase binding aptamer according to the present invention is preferably positioned within a distance of 10 to 1000 nt downstream of the transcription starting site, preferably within a distance of 50 to 500 nt downstream of the transcription starting site, more preferred within a distance of 60 to 250 nt downstream of the transcription starting site, particularly preferred within a distance of 80 to 103 nt downstream of the transcription starting site. The sequence encoding the transcription enhancing RNA-polymerase binding aptamer according to the present invention is preferably also operatively linked to the sequence to be expressed, e.g. an open reading frame. The distance of the transcription enhancing RNA-polymerase binding aptamer encoding sequence to the sequence to be expressed is not crucial. However, with certain distances superior effects in terms of regulating the transcription and expression may be observed. Hence, in one embodiment the sequence encoding the transcription enhancing RNA-polymerase binding aptamer according to the present invention is preferably positioned within a distance of 10 to 1000 nt upstream of the sequence to be expressed, or the multiple cloning site, preferably within a distance of 50 to 500 nt upstream of the sequence to be expressed, or the multiple cloning site, more preferred within a distance of 60 to 250 nt upstream of the sequence to be expressed, or the multiple cloning site, particularly preferred within a distance of 70 to 108 nt upstream of the sequence to be expressed, or the multiple cloning site. Any combination of the above mentioned preferred distances may be used, e.g. a distance of 10 to 1000 nt downstream of the transcription starting site and a distance of 10 to 1000 nt upstream of the sequence to be expressed, or the multiple cloning site, or distance of 50 to 500 nt downstream of the transcription starting site and a distance of 50 to 500 nt upstream of the sequence to be expressed, or the multiple cloning site, or a distance of 60 to 250 nt downstream of the transcription starting site and a distance of 60 to 250 nt upstream of the sequence to be expressed, or the multiple cloning site, of a distance of 80 to 103 nt downstream of the transcription starting site and a distance of 70 to 108 nt upstream of the sequence to be expressed, or the multiple cloning site, or any other possible combination. It has to be noted again that the term “transcription enhancing RNA-polymerase binding aptamer” refers to both, “activating RNA-polymerase binding aptamers” and “antiterminating RNA-polymerase binding aptamers”. Preferred transcription enhancing aptamers according to the present invention possess both features, i.e. are activating and antiterminating RNA-polymerase binding aptamers; particularly preferred are at least antiterminating RNA-polymerase binding aptamers.

It may further be advantageous to avoid hybridizing sequences in the region of the DNA sequence encoding the RNA-polymerase binding aptamer or the RNA-polymerase binding aptamer after transcription, such as reverse complement sequences. Without being bound by theory it is thought that such sequences under certain circumstances might hybridize to the DNA sequence or transcribed RNA-polymerase binding aptamer and thereby hinder it activity. Hence, in a preferred embodiment the DNA molecule or vector comprising the RNA-polymerase binding aptamer according to the present invention does not comprise a sequence that has 100% identity over a stretch of 20 nt or more. In a preferred embodiment the DNA molecule or vector does not include such hybridizing sequence on the same strand as the strand encoding the RNA-polymerase binding aptamer, preferably at least not on the strand section transcribed with the sequence encoding the RNA-polymerase binding aptamer. In a further preferred embodiment the DNA molecule or vector does not code for an RNA sequence hybridizing RNA-polymerase binding aptamer on the same RNA transcript, preferably at least not within a range of 30 nt surrounding the sequence encoding the RNA-polymerase binding aptamer is coded on.

Furthermore, the transcription enhancing RNA-polymerase binding aptamers exhibit anti-terminating activity (antiterminating RNA-polymerase binding aptamers), preferably when positioned upstream of a terminator sequence. In a preferred embodiment of the present invention the antiterminating RNA-polymerase binding aptamer is positioned upstream of a terminator sequence within the expression cassette. The terminator sequence may be positioned upstream, within or downstream of the sequence to be expressed. It may for example be part of the sequence encoding a protein of interest In such case it may be particularly desirable to include an antiterminating RNA-polymerase binding aptamer according to the present invention to allow sufficient expression of the protein without the need of interfering with the coding sequence comprising the terminator. Hence, the expression cassette according to the present invention preferably comprises a sequence encoding an antiterminating RNA-polymerase binding aptamer according to the present invention, preferably comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:72, SEQ ID NO:73, SEQ ID NO:74, and SEQ ID NO:75, more preferred selected from the group consisting of SEQ ID NO:2, SEQ ID NO:72, SEQ ID NO:73, SEQ ID NO:74, and SEQ ID NO:75. Embodiments of the sequences as outlined herein above apply likewise, e.g. with respect to identity.

The sequence encoding the antiterminating RNA-polymerase binding aptamer is preferably operatively linked to the terminator sequence, more preferably is positioned upstream of the terminator sequence. The distance of the antiterminating RNA-polymerase binding aptamer encoding sequence to the terminator sequence is not crucial. However, with certain distances superior effects in terms of regulating the transcription and expression may be observed. Hence, in one embodiment the sequence encoding the antiterminating RNA-polymerase binding aptamer according to the present invention is preferably positioned within a distance of 10 to 1000 nt upstream of the terminator sequence, preferably within a distance of 15 to 500 nt upstream of the terminator sequence, more preferred within a distance of 15 to 250 nt upstream of the terminator sequence, particularly preferred within a distance of 20 to 111 nt upstream of the terminator sequence. Any combination of the above mentioned preferred distances may be used. In one particular preferred embodiment the antiterminating RNA-polymerase binding aptamer coding sequence is positioned 80 to 103 nt downstream of the transcription starting site and 20 to 111 nt upstream of a terminator sequence. Preferred terminator sequences are selected from the group consisting of intrinsic terminator sequences, and factor dependent terminator sequences as further outlined herein.

In a particular preferred embodiment the expression cassette according to the present invention comprises a sequence encoding a transcription enhancing RNA-polymerase binding aptamer according to the present invention, a promoter and a multiple cloning site, wherein the sequence encoding said transcription enhancing RNA-polymerase binding aptamer is located downstream of the transcription starting site of said promoter and upstream of said multiple cloning site. The expression cassette may be comprised in a DNA-molecule, preferably comprised in a vector. Particularly preferred the transcription enhancing RNA-polymerase binding aptamer is an antiterminating RNA-polymerase binding aptamer according to the present invention.

Furthermore, the invention also relates to the use of a transcription enhancing RNA-polymerase binding aptamer according to the present invention or a DNA-molecule encoding the same for enhancing the expression of an endogenous sequence within a host cell. The host cell preferably being a non-human host cell more preferably bacteria, particularly preferred E. coli. The DNA-molecule encoding said transcription enhancing RNA-polymerase binding aptamer according to the present invention is preferably inserted downstream of the promoter from which said endogenous sequence is transcribed. The invention also relates to a method for enhancing the expression of an endogenous sequence in a host cell comprising the step of inserting a DNA-molecule comprising a sequence encoding an transcription enhancing RNA-polymerase binding aptamer according to the present invention into the genome of said host cell such that the sequence encoding said transcription enhancing RNA-polymerase binding aptamer according to the present invention is operatively linked to the promoter of said endogenous sequence, preferably inserted downstream of said promoter. Further preferred said aptamer is inserted downstream of said promoter and upstream of said endogenous sequence.

Furthermore, the invention relates to the use of antiterminating RNA-polymerase binding aptamer or a DNA-molecule comprising a sequence encoding the same for suppressing for the presence of a terminator in an endogenous sequence of a host cell.

The skilled person may choose the desired transcription enhancing RNA-polymerase binding aptamer, in particular regarding the strength of enhancement. He may consider certain aspects therefore, like strength of the promoter, i.e. the degree of transcription taking place without the aptamer. Furthermore, the skilled person will consider the amount of expression desired, e.g. upon induction of transcription. To this end, he may choose from the transcription enhancing RNA-polymerase binding aptamers provided herewith, as they have different degrees of enhancement. Furthermore, a plurality of sequences encoding transcription enhancing RNA-polymerase binding aptamers may be used. Hence, in one embodiment of the present invention the expression cassette comprises a sequence encoding two or more transcription enhancing RNA-polymerase binding aptamer, preferably three or more sequences encoding an transcription enhancing RNA-polymerase binding aptamer. The sequence comprises said two or more, or said three or more RNA-polymerase binding aptamer encoding sequences preferably as consecutive sequences, e.g. tandem repeats, optionally separated by spacer sequences. The aptamers encoded by said sequence may all be the same transcription enhancing RNA-polymerase binding aptamer or they may encode different transcription enhancing RNA-polymerase binding aptamers.

The transcription enhancing RNA-polymerase binding aptamers according to the present invention are applicable to a variety of uses and methods. The present invention, hence, relates to the use of transcription enhancing RNA-polymerase binding aptamers according to the present invention for regulating expression of nucleic acids, preferably to the use of a DNA sequence encoding a transcription enhancing RNA-polymerase binding aptamers according to the present invention for regulating expression of a sequence to be expressed. Furthermore, the present invention relates to the use of a DNA-molecule according to the present invention for enhancing expression of a sequence to be expressed. Furthermore, the invention relates to the use of an expression cassette according to the present invention for the expression of sequence of interest, preferably a protein. Likewise the invention relates to the use of a vector according to the present invention for the expression of a sequence of interest, preferably a protein. The invention likewise relates to the use of an expression cassette according to the present invention, i.e. comprising a sequence encoding the transcription enhancing RNA-polymerase binding aptamers according to the present invention, for expression of a sequence of interest, e.g. a sequence encoding a protein of interest. The expression may be performed in a host cell or in in vitro expression systems. The DNA-molecule, expression cassette and/or vector according to the present invention may used in either a host cell or an in vitro expression system.

In vitro expression systems are known by those of ordinary skills in the art and are widely used in the laboratory practice and the assays (see e.g. Spirin, A.; Baranov, V.; Ryabova, L.; Ovodov, S.; Alakhov, Y. (1988). “A continuous cell-free translation system capable of producing polypeptides in high yield”. Science 242 (4882): 1162; Jackson, A M et al. (2004). “Cell-free protein synthesis for proteomics”. Briefings in Functional Genomics and Proteomics 2 (4): 308-319; Carlson, E D et al. (2011). “Cell-free protein synthesis: Applications come of age”. Biotechnol Adv 30 (5): 1185; and Bundy, Bradley C.; Franciszkowicz, Marc J.; Swartz, James R. (2008). “Escherichia coli-based cell-free synthesis of virus-like particles”. Biotechnology and Bioengineering 100 (1): 28-37). One example of an in vitro expression system is PURExpress® In Vitro Protein Synthesis Kit (NEB; https://www.neb.com/products/e6800-purexpress-invitro-protein-synthesis-kit), which may be employed in connection with the present application. In vitro expression systems employ a DNA-dependent RNA polymerase for transcription of a DNA molecule into RNA and optionally may employ ribosomes for translation of the RNA into a polypeptide if desired. The systems are designed predominantly for the phage polymerases (T7, SP6, etc.) but may also be further optimised for the bacterial RNAP, preferred RNAP from E. coli. It will be apparent that the RNA-binding aptamer according to the invention preferably binds to the polymerase of the in vitro expression system, preferably with the affinities as outlined herein, preferably a non-phage polymerase, more preferably a bacterial RNA polymerase of the in vitro expression or transcription system. Nowadays, most of the in vitro systems use phage polymerases due to high yield of transcription. Nevertheless, they are prone to affect secondary structures in the transcribed RNAs and RNA misfolding. Currently in vitro systems with bacterial RNA polymerases are available but not commonly used, as the RNA yield is usually low. It is apparent to the skilled person that this is improved by the present invention. The invention hence in a preferred embodiment relates to the use of DNA-molecule according to the present invention, preferably comprising a sequence encoding an transcription enhancing RNA-polymerase binding aptamer according to the invention, for use in in vitro transcription and/or expression. Preferably the transcription enhancing RNA-polymerase binding aptamer binds to the bacterial RNA polymerase used for in vitro transcription and/or expression. The invention, furthermore, relates to a method for in vitro transcription of a RNA of interest comprising providing a DNA-molecule according to the present invention, the DNA-molecule comprising a sequence encoding RNA-polymerase binding aptamer according to the present invention, preferably the DNA-molecule comprising an expression cassette according to the present invention, said DNA-molecule comprising a sequence encoding the RNA of interest operatively linked to a promoter and the sequence encoding the RNA-polymerase binding aptamer, incubating said DNA-molecule with a RNA-polymerase according to the present invention under conditions allowing transcription from said promoter, and optionally recovering the RNA of interest. The RNA-polymerase binding aptamer may be a transcription enhancing RNA-polymerase binding aptamer or an inhibiting RNA-polymerase binding aptamer according to the present invention, and combinations thereof. The nature of the aptamer may be chosen according to the needs, e.g. whether the transcription is to be enhanced or inhibited. In a particular preferred embodiment the RNA-polymerase binding aptamer is a transcription enhancing RNA-polymerase binding aptamer according to the invention.

The skilled person knows conditions suited for in vitro transcription. They include the presence of nucleoside triphosphates (NTP) and suited buffers. Nucleoside triphosphade include natural nucleoside triphosphates, like adenosine triphosphate (ATP), guanosine triphosphate (GTP), cytidine triphosphate (CTP), 5-methyluridine triphosphate (m5UTP), and uridine triphosphate (UTP), but are not limited thereto. Furthermore, cofactors such as magnesium may be added. In one embodiment incubation is performed using a cell extract. Furthermore, transcription inhibitors may be added, e.g. Rho inhibitor of E. coli. This allows finer controlling of the expression.

Furthermore, the invention relates to a method for in vitro expression of a protein of interest comprising providing a DNA-molecule according to the present invention, the DNA-molecule comprising a sequence encoding a transcription enhancing RNA-polymerase binding aptamer according to the present invention, preferably the DNA-molecule comprising an expression cassette according to the present invention, said DNA-molecule comprising a sequence encoding the protein of interest operatively linked to a promoter and the sequence encoding a transcription enhancing RNA-polymerase binding aptamer, incubating said DNA-molecule with components and under conditions allowing transcription from said promoter and translation of the transcript, and optionally recovering the protein of interest. In vitro expression is also known as cell free expression. Common components of a cell free reaction include a cell extract, an energy source, a supply of amino acids, cofactors of the translation and/or translation machinery of the cell extract, and the DNA-molecule according to the invention. Such cofactors may be divalent cations, such as magnesium. A cell extract may be obtained by lysing the cell of interest and centrifuging out the cell walls, DNA genome, and other debris. The remains are the necessary cell machinery including ribosomes, aminoacyl-tRNA synthetases, translation initiation and elongation factors, nucleases, etc. Preferred cell extracts are extracts from cells outlined above as suited host cells according to the present invention, preferably eukaryotic or prokaryotic cell extracts, preferably bacterial, more preferably cell extracts of E. coli.

The invention also relates to the use of a DNA-molecule comprising a sequence encoding a transcription enhancing RNA-polymerase binding aptamer according to the present invention for increasing the yield of an in vitro transcription or an in vitro expression assay. This may for example be performed by the method as outlined above or by including said DNA-molecule or at least the sequence into an available assay.

Two types of DNA-molecules can be used in in vitro expression or transcription, i.e. plasmids and linear DNA-molecules (linear expression templates). Plasmids are circular, and are normally made using cells. Linear DNA-molecules can be made much more effectively via PCR (see polymerase chain reaction), which replicates DNA much faster than raising cells containing the plasmid. While Linear DNA-molecules are easier and faster to make, plasmid yields are usually much higher in cell free expression. Because of this, much research today is focused on optimizing cell free expression yields to approach the yields of plasmids when using the linear DNA-molecules. The present invention provides a solution.

An “energy source” refers to a needed energy source for transcription and/or translation, is added to the reaction, e.g. in addition to the cell extract. Preferred energy sources are phosphoenol pyruvate, acetyl phosphate, and creatine phosphate.

“Amino acids” as used herein include natural and non natural amino acids, preferably one or more of following proteinogenic amino acids are are added: Alanine, Cysteine, Aspartic acid, Glutamic acid, Phenylalanine, Glycine, Histidine, Isoleucine, Lysine, Leucine, Methionine, Asparagine, Pyrrolysine, Proline, Glutamine, Arginine, Serine, Threonine, Selenocysteine, Valine, Tryptophan, and Tyrosine. In one embodiment all of the listed amino acids are added.

The invention, hence, also pertains kit comprising a DNA-molecule or vector according to the present invention. The kit may comprise further components usable for the respective assay they are intended for, e.g. components of an in vitro transcription or expression system, or reagents for transfection or transformation of a host cell, buffer or further reagents, or host cells as further described herein.

By using the transcription enhancing RNA-polymerase binding aptamers according to the present invention expression of sequences can be enhanced, e.g. sequences encoding a protein of interest. Hence, the present invention in a particular preferred embodiment relates to a method of producing a protein of interest comprising providing a vector according to the present invention, the vector comprising a sequence encoding a transcription enhancing RNA-polymerase binding aptamer according to the present invention, preferably the vector comprising an expression cassette according to the present invention, cloning the sequence encoding said protein of interest into said vector such that the sequence is operatively linked to the promoter and the sequence encoding the transcription enhancing RNA-polymerase binding aptamer to obtain the final expression vector, introducing said expression vector into a host cell, and culturing said host cell in culture medium under conditions inducing transcription from the promoter of the expression vector, and optionally recovering the protein of interest from the host cell or culture medium. It will be understood by the skilled artisan that the cloning step of the method of producing a protein of interest may be omitted if the initial vector comprises the sequence encoding said protein of interest. In this embodiment the method according to the present invention comprises the steps of providing a vector according to the present invention, preferably comprising an expression cassette according to the present invention, said vector comprising a sequence encoding the protein of interest operatively linked to the promoter and the sequence encoding a transcription enhancing RNA-polymerase binding aptamer, introducing said vector into a host cell, and culturing said host cell in culture medium under conditions inducing transcription from the promoter of the expression vector, and optionally recovering the protein of interest from the host cell or culture medium. The transcription enhancing RNA-polymerase binding aptamer may be an activating or an antiterminating RNA-polymerase binding aptamer, preferably an antiterminating according to the present invention and embodiments outlined above.

It has been found by the inventors that a subgroup of the RNA-polymerase binding aptamers according to the present invention decrease the activity of RNA polymerase when transcribed from a DNA strand encoding said aptamer. The efficiency of transcription of a DNA segment is decreased if the DNA segment comprises a sequence encoding a RNA-polymerase binding aptamer according to the invention. Hence, in one preferred embodiment of the present invention the RNA-polymerase binding aptamer decreases (reduces or inhibits) the activity of a RNA-polymerase. The RNA-polymerase binding aptamers according to this embodiment are also referred to interchangeably as “inhibiting” or “transcription inhibiting” RNA-polymerase binding aptamers. The skilled person is aware of methods, assays and systems to determine activity of RNA-polymerases and effects of sequences thereon. Such methods include the expression of reporter genes in a host cell or may apply in vitro transcription assays as outlined in detail herein above and in the enclosed Examples.

In one preferred embodiment the RNA-polymerase binding aptamer according the present invention inhibits the activity of the RNA-polymerase, preferably in a reporter assay as disclosed herein. The inhibition may be Rho-dependent or Rho-independent, preferably Rho-dependent.

In a preferred embodiment of the present invention the inhibiting RNA-polymerase binding aptamer decreases or reduces expression of a sequence to be expressed, wherein the reduction of expression is at least 30% as compared to the expression of said sequence to be expressed when not comprising said inhibiting RNA-polymerase binding aptamer, preferably by at least 20%, even more preferably by at least 50%.

The inhibiting RNA-polymerase binding aptamer according to the present invention preferably comprises a transcription pausing motif, preferably with the consensus sequence of G₋₁₀Y₋₁G₊₁.

Furthermore, it has been found by the inventors that the inhibiting RNA-polymerase binding aptamers have an increased C-content median value of 33.67%, whereas G-content median value is 15.69% (FIG. 19). Hence, in one embodiment the inhibitory RNA-polymerase binding aptamer has a C-content of more than 27%, preferably more than 30%, more preferably more than 31%, and wherein the RNA-polymerase binding aptamer has a G-content of less than 23%, preferably less than 20%, more preferably less than 17%.

Consensus sequences have been identified which are common to different inhibiting RNA-polymerase binding aptamers. Hence, in a preferred embodiment of the inhibiting RNA-polymerase binding aptamer according to the invention, the aptamer comprises a sequence as encoded by a sequence selected from the group consisting of SEQ ID NO:84, SEQ ID NO:85, SEQ ID NO:86, and SEQ ID NO:87.

Different sequences fall within these consensus sequences; see Example 2. Hence, in a preferred embodiment of the inhibiting RNA-polymerase binding aptamer according to the invention, the aptamer comprises a sequence as encoded by a sequence of SEQ ID NO:84, and comprises as sequence encoded by a sequence having at least 80% identity to a sequence selected from the group consisting of SEQ ID NO:29, SEQ ID NO:35, SEQ ID NO:41, SEQ ID NO:27, and SEQ ID NO:39, preferably encoded by a sequence having at least 90% identity to a sequence selected from the group consisting of SEQ ID NO:29, SEQ ID NO:35, SEQ ID NO:41, SEQ ID NO:27, and SEQ ID NO:39; more preferably encoded by a sequence having at least 95% identity to a sequence selected from the group consisting of SEQ ID NO:29, SEQ ID NO:35, SEQ ID NO:41, SEQ ID NO:27, and SEQ ID NO:39; yet further preferred encoded by a sequence having at least 99% identity to a sequence selected from the group consisting of SEQ ID NO:29, SEQ ID NO:35, SEQ ID NO:41, SEQ ID NO:27, and SEQ ID NO:39. In a certain embodiment the inhibiting RNA-polymerase binding aptamer according to the invention comprises a sequence as encoded by a sequence of SEQ ID NO:84, and comprises a sequence encoded by a sequence selected from the group consisting of SEQ ID NO:29, SEQ ID NO:35, SEQ ID NO:41, SEQ ID NO:27, and SEQ ID NO:39.

In a further preferred embodiment of the inhibiting RNA-polymerase binding aptamer according to the invention, the aptamer comprises a sequence as encoded by a sequence of SEQ ID NO:85, and comprises as sequence encoded by a sequence having at least 80% identity to a sequence selected from the group consisting of SEQ ID NO:31, SEQ ID NO:91, SEQ ID NO:88, SEQ ID NO:32, SEQ ID NO:26, and SEQ ID NO:36, preferably encoded by a sequence having at least 90% identity to a sequence selected from the group consisting of SEQ ID NO:31, SEQ ID NO:91, SEQ ID NO:88, SEQ ID NO:32, SEQ ID NO:26, and SEQ ID NO:36; more preferably encoded by a sequence having at least 95% identity to a sequence selected from the group consisting of SEQ ID NO:31, SEQ ID NO:91, SEQ ID NO:88, SEQ ID NO:32, SEQ ID NO:26, and SEQ ID NO:36; yet further preferred encoded by a sequence having at least 99% identity to a sequence selected from the group consisting of SEQ ID NO:31, SEQ ID NO:91, SEQ ID NO:88, SEQ ID NO:32, SEQ ID NO:26, and SEQ ID NO:36. In a certain embodiment the inhibiting RNA-polymerase binding aptamer according to the invention comprises a sequence as encoded by a sequence of SEQ ID NO:85, and comprises a sequence encoded by a sequence selected from the group consisting of SEQ ID NO:31, SEQ ID NO:91, SEQ ID NO:88, SEQ ID NO:32, SEQ ID NO:26, and SEQ ID NO:36.

In a further preferred embodiment of the inhibiting RNA-polymerase binding aptamer according to the invention, the aptamer comprises a sequence as encoded by a sequence of SEQ ID NO:86, and comprises as sequence encoded by a sequence having at least 80% identity to a sequence selected from the group consisting of SEQ ID NO:89, and SEQ ID NO:34, preferably encoded by a sequence having at least 90% identity to a sequence selected from the group consisting of SEQ ID NO:89, and SEQ ID NO:34; more preferably encoded by a sequence having at least 95% identity to a sequence selected from the group consisting of SEQ ID NO:89, and SEQ ID NO:34; yet further preferred encoded by a sequence having at least 99% identity to a sequence selected from the group consisting of SEQ ID NO:89, and SEQ ID NO:34. In a certain embodiment the inhibiting RNA-polymerase binding aptamer according to the invention comprises a sequence as encoded by a sequence of SEQ ID NO:86, and comprises a sequence encoded by a sequence selected from the group consisting of SEQ ID NO:89, and SEQ ID NO:34.

In a further preferred embodiment of the inhibiting RNA-polymerase binding aptamer according to the invention, the aptamer comprises a sequence as encoded by a sequence of SEQ ID NO:87, and comprises as sequence encoded by a sequence having at least 80% identity to a sequence selected from the group consisting of SEQ ID NO:37, SEQ ID NO:40, SEQ ID NO:33, SEQ ID NO:25; SEQ ID NO:92, SEQ ID NO:30, SEQ ID NO:90, and SEQ ID NO:38, preferably encoded by a sequence having at least 90% identity to a sequence selected from the group consisting of SEQ ID NO:37, SEQ ID NO:40, SEQ ID NO:33, SEQ ID NO:25; SEQ ID NO:92, SEQ ID NO:30, SEQ ID NO:90, and SEQ ID NO:38; more preferably encoded by a sequence having at least 95% identity to a sequence selected from the group consisting of SEQ ID NO:37, SEQ ID NO:40, SEQ ID NO:33, SEQ ID NO:25; SEQ ID NO:92, SEQ ID NO:30, SEQ ID NO:90, and SEQ ID NO:38; yet further preferred encoded by a sequence having at least 99% identity to a sequence selected from the group consisting of SEQ ID NO:37, SEQ ID NO:40, SEQ ID NO:33, SEQ ID NO:25; SEQ ID NO:92, SEQ ID NO:30, SEQ ID NO:90, and SEQ ID NO:38. In a certain embodiment the inhibiting RNA-polymerase binding aptamer according to the invention comprises a sequence as encoded by a sequence of SEQ ID NO:87, and comprises a sequence encoded by a sequence selected from the group consisting of SEQ ID NO:37, SEQ ID NO:40, SEQ ID NO:33, SEQ ID NO:25; SEQ ID NO:92, SEQ ID NO:30, SEQ ID NO:90, and SEQ ID NO:38.

The inventors found a plurality of inhibiting RNA-polymerase binding aptamers which decrease the activity of the RNA-polymerase they are binding to. Hence, the inhibiting RNA-polymerase binding aptamer according to the present invention is in one embodiment encoded by a sequence having at least 80% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a further embodiment the inhibiting RNA-polymerase binding aptamer according to the present invention is encoded by a sequence having at least 90% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In yet a further embodiment the inhibiting RNA-polymerase binding aptamer according to the present invention is encoded by a sequence having at least 95% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a preferred embodiment the inhibiting RNA-polymerase binding aptamer according to the present invention is encoded by a sequence having at least 99% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. In a particular embodiment the inhibiting RNA-polymerase binding aptamer according to the present invention is encoded by a sequence having a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92. The invention also relates to a nucleic acid hybridizing to any of the above sequences under stringent conditions.

In a preferred embodiment of the present invention the inhibiting RNA-polymerase binding aptamer according to the present invention is encoded by a sequence having at least 80% identity to SEQ ID NO:25, preferably by a sequence having at least 90% identity, more preferably by a sequence having at least 95% identity, even more preferred by a sequence having at least 99% identity to SEQ ID NO:25. In a particular embodiment the inhibiting RNA-polymerase binding aptamer is encoded by the sequence of SEQ ID NO:25. In a preferred embodiment of the inhibiting RNA-polymerase binding aptamer being encoded by a sequence having a certain identity to SEQ ID NO:25, the sequence encoding the aptamer is not altered at the nucleotides at positions corresponding to 10, 11, 18, 20, 21, and 24 to 27 of SEQ ID NO:25.

In a preferred embodiment of the present invention the inhibiting RNA-polymerase binding aptamer according to the present invention is encoded by a sequence having at least 80% identity to SEQ ID NO:26, preferably by a sequence having at least 90% identity, more preferably by a sequence having at least 95% identity, even more preferred by a sequence having at least 99% identity to SEQ ID NO:26. In a particular embodiment the inhibiting RNA-polymerase binding aptamer is encoded by the sequence of SEQ ID NO:26.

In a preferred embodiment of the present invention the inhibiting RNA-polymerase binding aptamer according to the present invention is encoded by a sequence having at least 80% identity to SEQ ID NO:27, preferably by a sequence having at least 90% identity, more preferably by a sequence having at least 95% identity, even more preferred by a sequence having at least 99% identity to SEQ ID NO:27. In a particular embodiment the inhibiting RNA-polymerase binding aptamer is encoded by the sequence of SEQ ID NO:27.

In a preferred embodiment of the present invention the inhibiting RNA-polymerase binding aptamer according to the present invention is encoded by a sequence having at least 80% identity to SEQ ID NO:28, preferably by a sequence having at least 90% identity, more preferably by a sequence having at least 95% identity, even more preferred by a sequence having at least 99% identity to SEQ ID NO:28. In a particular embodiment the inhibiting RNA-polymerase binding aptamer is encoded by the sequence of SEQ ID NO:28.

In a preferred embodiment of the present invention the inhibiting RNA-polymerase binding aptamer according to the present invention is encoded by a sequence having at least 80% identity to SEQ ID NO:29, preferably by a sequence having at least 90% identity, more preferably by a sequence having at least 95% identity, even more preferred by a sequence having at least 99% identity to SEQ ID NO:29. In a particular embodiment the inhibiting RNA-polymerase binding aptamer is encoded by the sequence of SEQ ID NO:29.

In a preferred embodiment of the present invention the inhibiting RNA-polymerase binding aptamer according to the present invention is encoded by a sequence having at least 80% identity to SEQ ID NO:30, preferably by a sequence having at least 90% identity, more preferably by a sequence having at least 95% identity, even more preferred by a sequence having at least 99% identity to SEQ ID NO:30. In a particular embodiment the inhibiting RNA-polymerase binding aptamer is encoded by the sequence of SEQ ID NO:30.

In a preferred embodiment of the present invention the inhibiting RNA-polymerase binding aptamer according to the present invention is encoded by a sequence having at least 80% identity to SEQ ID NO:31, preferably by a sequence having at least 90% identity, more preferably by a sequence having at least 95% identity, even more preferred by a sequence having at least 99% identity to SEQ ID NO:31. In a particular embodiment the inhibiting RNA-polymerase binding aptamer is encoded by the sequence of SEQ ID NO:31.

In a preferred embodiment of the present invention the inhibiting RNA-polymerase binding aptamer according to the present invention is encoded by a sequence having at least 80% identity to SEQ ID NO:32, preferably by a sequence having at least 90% identity, more preferably by a sequence having at least 95% identity, even more preferred by a sequence having at least 99% identity to SEQ ID NO:32. In a particular embodiment the inhibiting RNA-polymerase binding aptamer is encoded by the sequence of SEQ ID NO:32.

In a preferred embodiment of the present invention the inhibiting RNA-polymerase binding aptamer according to the present invention is encoded by a sequence having at least 80% identity to SEQ ID NO:33, preferably by a sequence having at least 90% identity, more preferably by a sequence having at least 95% identity, even more preferred by a sequence having at least 99% identity to SEQ ID NO:33. In a particular embodiment the inhibiting RNA-polymerase binding aptamer is encoded by the sequence of SEQ ID NO:33.

In a preferred embodiment of the present invention the inhibiting RNA-polymerase binding aptamer according to the present invention is encoded by a sequence having at least 80% identity to SEQ ID NO:34, preferably by a sequence having at least 90% identity, more preferably by a sequence having at least 95% identity, even more preferred by a sequence having at least 99% identity to SEQ ID NO:34. In a particular embodiment the inhibiting RNA-polymerase binding aptamer is encoded by the sequence of SEQ ID NO:34.

In a preferred embodiment of the present invention the inhibiting RNA-polymerase binding aptamer according to the present invention is encoded by a sequence having at least 80% identity to SEQ ID NO:35, preferably by a sequence having at least 90% identity, more preferably by a sequence having at least 95% identity, even more preferred by a sequence having at least 99% identity to SEQ ID NO:35. In a particular embodiment the inhibiting RNA-polymerase binding aptamer is encoded by the sequence of SEQ ID NO:35.

In a preferred embodiment of the present invention the inhibiting RNA-polymerase binding aptamer according to the present invention is encoded by a sequence having at least 80% identity to SEQ ID NO:36, preferably by a sequence having at least 90% identity, more preferably by a sequence having at least 95% identity, even more preferred by a sequence having at least 99% identity to SEQ ID NO:36. In a particular embodiment the inhibiting RNA-polymerase binding aptamer is encoded by the sequence of SEQ ID NO:36.

In a preferred embodiment of the present invention the inhibiting RNA-polymerase binding aptamer according to the present invention is encoded by a sequence having at least 80% identity to SEQ ID NO:37, preferably by a sequence having at least 90% identity, more preferably by a sequence having at least 95% identity, even more preferred by a sequence having at least 99% identity to SEQ ID NO:37. In a particular embodiment the inhibiting RNA-polymerase binding aptamer is encoded by the sequence of SEQ ID NO:37.

In a preferred embodiment of the present invention the inhibiting RNA-polymerase binding aptamer according to the present invention is encoded by a sequence having at least 80% identity to SEQ ID NO:38, preferably by a sequence having at least 90% identity, more preferably by a sequence having at least 95% identity, even more preferred by a sequence having at least 99% identity to SEQ ID NO:38. In a particular embodiment the inhibiting RNA-polymerase binding aptamer is encoded by the sequence of SEQ ID NO:38.

In a preferred embodiment of the present invention the inhibiting RNA-polymerase binding aptamer according to the present invention is encoded by a sequence having at least 80% identity to SEQ ID NO:39, preferably by a sequence having at least 90% identity, more preferably by a sequence having at least 95% identity, even more preferred by a sequence having at least 99% identity to SEQ ID NO:39. In a particular embodiment the inhibiting RNA-polymerase binding aptamer is encoded by the sequence of SEQ ID NO:39.

In a preferred embodiment of the present invention the inhibiting RNA-polymerase binding aptamer according to the present invention is encoded by a sequence having at least 80% identity to SEQ ID NO:40, preferably by a sequence having at least 90% identity, more preferably by a sequence having at least 95% identity, even more preferred by a sequence having at least 99% identity to SEQ ID NO:40. In a particular embodiment the inhibiting RNA-polymerase binding aptamer is encoded by the sequence of SEQ ID NO:40.

In a preferred embodiment of the present invention the inhibiting RNA-polymerase binding aptamer according to the present invention is encoded by a sequence having at least 80% identity to SEQ ID NO:41, preferably by a sequence having at least 90% identity, more preferably by a sequence having at least 95% identity, even more preferred by a sequence having at least 99% identity to SEQ ID NO:41. In a particular embodiment the inhibiting RNA-polymerase binding aptamer is encoded by the sequence of SEQ ID NO:41.

All embodiments as outlined above in general for the DNA-molecule, the expression cassette, the vector and the host cell apply likewise to the preferred embodiments of the inhibiting RNA-polymerase binding aptamers.

It will also be acknowledged by the skilled person that further regulation of expression of endogenous or exogenous sequence using the RNA-polymerase binding aptamers according to the present invention may be conducted by employing the dependency of the found mechanism on the Rho inhibition factor. When for example employing an inhibiting RNA-polymerase binding aptamer according to the present invention its inhibiting effect on the expression may controlled by the incubation of the expression system (in vitro or in vivo) with an inhibitor of Rho inhibitor, e.g. bicyclomycin. This allows a fine control of expression. Hence, in one embodiment of the uses and methods according to the present invention, an inhibitor of Rho transcription inhibitor is added to further regulate expression. The inhibitor or Rho transcription inhibitor according to the present invention is preferably bicyclomycin.

This is particularly useful in connection with the inhibiting RNA-polymerase binding aptamers according to the present invention. Hence, in one embodiment the invention relates to a method for controlled expression of a sequence of interest in a host cell comprising the steps of: providing a host cell comprising a DNA-molecule, said DNA molecule comprising a sequence encoding for an inhibiting RNA-polymerase binding aptamer according to the present invention operatively linked to a promoter and the sequence of interest, culturing said host cell in culture medium under conditions allowing expression of the sequence of interest at the desired level, wherein said culture medium comprises an inhibitor of Rho transcription inhibitor at a concentration allowing the expression at the desired level. The sequence of interest may be an exogenous sequence or an endogenous sequence of the host cell. Hence, the method may for example be used for controlled down-regulation (inhibition) of endogenous sequence of a host cell, e.g. for functional analysis. Any inhibiting RNA-polymerase according to the invention may be used, preferably those as defined above, as all tested inhibiting RNA-polymerase binding aptamers according to the invention have shown significant dependency on Rho inhibitor and bicyclomycin (BCM). Hence, in a preferred embodiment in this regard, the inhibiting RNA-polymerase binding aptamer is encoded by a sequence as defined herein above. However, preferred inhibiting RNA-polymerase binding aptamer is encoded by a sequence having at least 80% identity to a sequence selected from the group consisting of SEQ ID NO:25, and SEQ ID NO:92, preferably having at least 90% identity to a sequence selected from the group consisting of SEQ ID NO:25, and SEQ ID NO:92, more preferably having at least 95% identity to a sequence selected from the group consisting of SEQ ID NO:25, and SEQ ID NO:92, more the inhibiting RNA-polymerase binding aptamer is encoded by a sequence selected from the group consisting of SEQ ID NO:25, and SEQ ID NO:92.

The inhibitor of Rho transcription inhibitor according to the present invention is preferably bicyclomycin, preferably at a level of 1 μg/mL to 100 μg/mL; more preferably 1 μg/mL to 50 μg/mL, most preferably 5 μg/mL to 25 μg/mL, including 8 μg/mL, 10 μg/mL, 15 p/mL, 20 μg/ml, 24 μg/mL.

As outline herein above, the invention also relates to a DNA-molecule comprising a sequence encoding a RNA-polymerase binding aptamer according to the present invention. In a preferred embodiment the RNA-polymerase binding aptamer is an inhibiting RNA-polymerase binding aptamer, preferably selected from the group of inhibiting RNA-polymerase aptamers as listed herein above. As will be understood by the person skilled in the art, a DNA-molecule comprising a sequence encoding an inhibiting RNA-polymerase binding aptamer may preferably be used for repressing or inhibiting expression from a promoter to which it is operatively linked. Hence, in a preferred embodiment of the invention the DNA-molecule according to the present invention comprises a sequence encoding an inhibiting RNA-polymerase binding aptamer and a promoter, wherein the sequence encoding said aptamer is operatively linked to said promoter, preferably operatively linked to repress or reduce transcription from said promoter. Several approaches make use of promoters which from which transcription is only active or increased under certain conditions, like the presence of a certain compound or other environmental conditions, such as temperature. However, these approaches often suffer from the fact that the inducible promoters are not completely “tight” under non-induction conditions, i.e. basal transcription and expression occurring from the promoter even in the absence of the stimulus for inducing transcription from the promoter. This, however, may be deleterious to the host cell, particularly in the case of proteins of interest having deleterious properties. Under these circumstances it is desirable to provide for an expression system or cassette which is “tight” under non-induction conditions. The inventors now provide inhibiting RNA-polymerase binding aptamers which provide unexpected effects in this regard. By including a sequence encoding such inhibiting RNA-polymerase binding aptamer operatively linked to the promoter, it is possible to tighten the promoter. Hence, in a preferred embodiment the present invention relates to DNA-molecule comprising an expression cassette, said expression cassette comprising an inducible promoter according to the invention, and a sequence encoding an inhibiting RNA-polymerase binding aptamer according to the invention, wherein the sequence encoding the aptamer is operatively linked to said inducible promoter, preferably positioned downstream of said inducible promoter. The skilled person may choose the desired inhibiting RNA-polymerase binding aptamer, in particular regarding the strength of inhibition. He may consider certain aspects therefore, like leakiness of the promoter in the non-induced state, i.e. the degree of transcription taking place without a stimulus. Furthermore, the skilled person will consider the amount of expression desired upon induction of transcription. To this end, he may choose from the inhibiting RNA-polymerase binding aptamers provided herewith, as they have different degrees of inhibition. Furthermore, a plurality of sequences encoding inhibiting RNA-polymerase binding aptamers may be used. Hence, in one embodiment of the present invention the expression cassette comprises a sequence encoding two or more inhibiting RNA-polymerase binding aptamers, preferably three or more inhibiting RNA-polymerase binding aptamers. These aptamers may all be the same inhibiting RNA-polymerase binding aptamer or they may be different inhibiting RNA-polymerase binding aptamers according to the invention.

The present invention in one embodiment relates to the use of a sequence encoding an inhibiting RNA-polymerase binding aptamer according to the present invention for inhibiting expression of a protein, preferably for inhibiting expression of a protein in a microorganism, such as prokaryotic or eukaryotic cell lines, including bacteria and mammal cell lines. In a preferred embodiment of the present invention said sequence encoding said RNA-polymerase binding aptamer is positioned downstream of the promoter from which the expression of said protein is initiated. This means that the RNA-polymerase binding aptamer is part of the mRNA encoding the protein to be expressed. The use according to the present invention encompasses the use for regulating the expression of any protein of interest.

The invention also relates to the use, or a method of use of an inhibiting RNA-polymerase binding aptamer according to the present invention or a DNA-molecule encoding the same for inhibiting the expression of an endogenous sequence within a host cell. The host cell preferably being a non-human host cell. The DNA-molecule encoding said inhibiting RNA-polymerase binding aptamer according to the present invention is preferably inserted downstream of the promoter from which said endogenous sequence is transcribed. The invention also relates to a method for inhibiting the expression of an endogenous sequence in a host cell comprising the step of inserting a DNA-molecule comprising a sequence encoding an inhibiting RNA-polymerase binding aptamer according to the present invention into the genome of said host cell such that the sequence encoding said inhibiting RNA-polymerase binding aptamer according to the present invention is operatively linked to the promoter of said endogenous sequence, preferably inserted downstream of said promoter. Further preferred said aptamer is inserted downstream of said promoter and upstream of said endogenous sequence.

The sequence encoding the inhibiting RNA-polymerase binding aptamer is preferably operatively linked to a promoter and/or a sequence to be expressed, or a multiple cloning site. In particularly preferred embodiment of the present invention, the sequence encoding the inhibiting RNA-polymerase binding aptamer is operatively linked to a promoter, and optionally to a sequence to be expressed, or a multiple cloning site. It has been shown herein, that the inhibiting RNA-polymerase binding aptamers are able to decrease transcription/expression of a transcript comprising the aptamer. The sequence encoding the inhibiting RNA-polymerase binding aptamer according to the present invention is preferably operatively linked to the promoter sequence and the transcription starting site. It is preferably positioned downstream of the promoter and the transcription starting site on the DNA-molecule according to the present invention. The distance of the inhibiting RNA-polymerase binding aptamer encoding sequence to the transcription starting site is not crucial. However, with certain distances superior effects in terms of regulating and inhibiting the transcription may be observed. Hence, in one embodiment the sequence encoding the inhibiting RNA-polymerase binding aptamer according to the present invention is preferably positioned within a distance of 10 to 1000 nt downstream of the transcription starting site, preferably within a distance of 15 to 500 nt downstream of the transcription starting site, more preferred within a distance of 25 to 250 nt downstream of the transcription starting site. The sequence encoding the inhibiting RNA-polymerase binding aptamer according to the present invention is preferably also operatively linked to the sequence to be expressed, e.g. an open reading frame, or a multiple cloning site. The distance of the inhibiting RNA-polymerase binding aptamer encoding sequence to the sequence to be expressed is not crucial. However, with certain distances superior effects in terms of regulating and inhibiting the transcription and expression may be observed. Hence, in one embodiment the sequence encoding the inhibiting RNA-polymerase binding aptamer according to the present invention is preferably positioned at least 20 nt upstream of the sequence to be expressed, or the multiple cloning site, e.g. within a distance of 20 to 1000 nt upstream of the sequence to be expressed, or the multiple cloning site, preferably within a distance of 30 to 500 nt upstream of the sequence to be expressed, or the multiple cloning site, more preferred within a distance of 40 to 250 nt upstream of the sequence to be expressed, or the multiple cloning site. Any combination of the above mentioned preferred distances may be used, e.g. a distance of 10 to 1000 nt downstream of the transcription starting site and a distance of 20 to 1000 nt upstream of the sequence to be expressed, or distance of 15 to 500 nt downstream of the transcription starting site and a distance of 15 to 500 nt upstream of the sequence to be expressed, or a distance of 25 to 250 nt downstream of the transcription starting site and a distance of 40 to 250 nt upstream of the sequence to be expressed, or any other possible combination.

The inventors have found that one mechanism of function is temporarily uncoupling transcription from translation. Upon binding to the surface of RNA polymerase, the inhibiting RNA-binding aptamer create a physical barrier to ribosome progression (FIG. 23E); see e.g. FIG. 23E. Hence, in one embodiment the inhibiting RNA-polymerase binding aptamer according to the invention is encoded within the sequence to be expressed, preferably within the ORF of a protein to be expressed. In one embodiment the inhibiting RNA-polymerase binding aptamer is encoded by the sequence of SEQ ID NO:25 or a sequence having at least 80% identity to the sequence of SEQ ID NO:25, preferably at least 90% identity, more preferably at least 95% identity, yet more preferred at least 99% identity.

The invention also relates to a method of producing a protein of interest comprising the steps of: providing a vector comprising an expression cassette according to the present invention, the expression cassette comprising a sequence encoding an inhibiting RNA-polymerase binding aptamer according to the present invention, introducing said vector into a host cell, and culturing said host cell in culture medium under conditions inducing transcription from the promoter of the expression cassette, and optionally recovering the protein of interest from the host cell or the the culture medium. The promoter preferably is selected from constitutive promoters or inducible promoters according to the invention, preferably being an inducible promoter according to the invention. Further, the protein to be expressed preferably having deleterious effects on said host cell. The method may comprise the step of culturing said host cell under conditions not inducing transcription from the inducible promoter of the expression cassette before the step of culturing said host cell in culture medium under conditions inducing transcription from the inducible promoter of the expression cassette.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

### General Materials and Methods and Identification of RNA-Polymerase Interacting Aptamers (RAP)

### Deep Sequencing

The RNA library from SELEX cycle 7 was first reverse transcribed to cDNA and amplified by 16 cycles of PCR with Phusion polymerase (New England Biolabs) using primers with fixed part and added barcode for multiplexing according to P. Parameswaran et al., Nucleic Acids Res. 35, e130 (2007) and N. Windbichler, F. von Pelchrzim, O. Mayer, E. Csaszar, R. Schroeder, RNA Biol. 5, 30-40 (2008) (fixFW: TATAGGGGAATTCGGAGCGGG (SEQ ID NO:45, fixRV_multi: TAGCCCGGGATCCTCGGGGCTG (Sigma; SEQ ID NO:46)). Library preparation (adapter ligation step) and deep sequencing were performed at the CSF NGS unit http://csf.ac.at/. The SELEX library was multiplexed and sequenced with the Solexa technology on a GAIIx with paired-end 76 base-pair reads. Reads were trimmed using trimmomatic (A. M. Bolger, M. Lohse, B. Usadel, Bioinformatics. 30, 2114-20 (2014)) and mapped against the reference genomes of the E. coli strain K12 (substrain MG1655, GenBank ID: U00096.3) using NextGenMap (F. J. Sedlazeck, P. Rescheneder, A. von Haeseler, Bioinformatics. 29, 2790-1 (2013)). Read mapping was conducted with permissive settings that allowed up to 20% sequence divergence in order to account for the considerable amounts of sequence alterations introduced by the SELEX procedure (B. Zimmermann, I. Bilusic, C. Lorenz, R. Schroeder, Methods. 52, 125-32 (2010); and B. Zimmermann, T. Gesell, D. Chen, C. Lorenz, R. Schroeder, PLoS One, 5, e9169 (2010)). The mapped reads were then filtered for a minimum mapping quality of 20, which resulted in 915,594 paired-end reads. Corresponding ORF annotations were downloaded from EcoGene 3.0 (J. Zhou, K. E. Rudd, Nucleic Acids Res. 41, D613-24 (2013)) and 5′ and 3′ UTR annotations—from RegulonDB (J. Zhou, K. E. Rudd, Nucleic Acids Res, 41, D613-24 (2013)).

### Peak Finding

Strand-specific coverage signals from K12 alignments were extracted and a custom peak-finding method was applied to these signals in order to identify the originating genomic regions of mRNA-fragments that were found to be enriched in the respective SELEX cycle. Briefly, our peak-calling method consists of the following steps: (1) signal smoothing using a moving Gaussian kernel, (2) approximation of the first derivative at each signal position by cubic Hermite interpolation, (3) calling peaks based on derivative sign-changes and defined minimum/maximum peak dimensions (minimum width: 10 bp, maximum width: 500 bp, minimum (smoothed) peak height: T. L. Bailey et al., Nucleic Acids Res. 37, W202-8 (2009)).

### Interval Annotation and Categorization

Peak intervals were classified according to whether they overlap with annotated ORFs or UTRs. Intervals were annotated as being “antisense” when they mapped to the strand opposite an existing ORF or UTR annotation. Based on the genomic location they were classified into several categories: 5′UTR (corresponding sequence aligns within any annotated 5′UTR), 3′UTR (corresponding sequence aligns within any annotated 3′UTR), intragenic (sense) (corresponding sequence aligns within the annotated gene, excluding cases of 5′- and 3′UTR), antisense (corresponding sequence aligns on the strand opposite to the annotated gene) and intergenic (sense) (corresponding sequence in between the annotated genes).

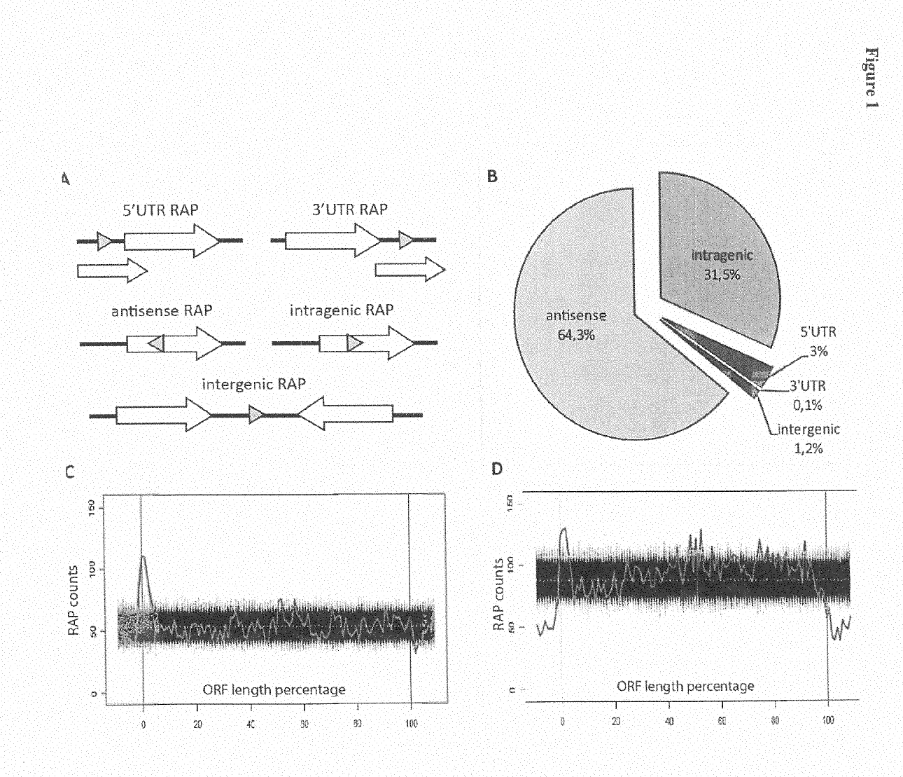

In order to characterize the location of RAPs within a transcript, their relative positioning was calculated within annotated transcripts (FIG. 1C-1D). The data indicate enrichment of RAPs around the translation start site and a depletion of RAPs in the 5′/3′ UTR regions. Additionally, a slight enrichment of RAPs around the midpoint of the transcripts and in the second ORF half for antisense RAPs was observed.

### Motif Search in RAPs

Motifs in the RAP sequences were identified using MEME algorithm (T. L. Bailey et al., Nucleic Acids Res. 37, W202-8 (2009)). Using default settings a (CAN)n motif was identified with a length of 29 bp in 1010 of the total identified 15,724 RAPs (6.4%). The vast majority (89%) of the RAPs containing this motif are anti-sense to an annotated ORF. Homer (S. Heinz et al., Mol. Cell. 38, 576-89 (2010)), another motif discovery tool, confirmed the (CAN)n motif when run with a motif length of 30 bp in 1909 of the 15,724 RAPs (12.14%).

Additionally, the RAPs were analyzed for the consensus RNA polymerase pausing sequence (see M. H. Larson et al., Science, 344, 1042-1047 (2014); and I. O. Vvedenskaya et al., Science, 344, 1285-1289 (2014)). The G₋₁₀Y₋₁G₊₁ motif was identified in 4656 (out of 5439) sense and in 8324 (out of 9956) anti-sense RAPs. To assess whether the motif is enriched, a Monte Carlo simulation was performed where we randomly shuffled the positions of the detected RAPs on the genome (keeping number, length and orientation of the RAPs constant) and counted the number of occurrences of the G₋₁₀Y₋₁G₊₁ motif in this random set for each trail. 10,000 trails were performed separately for sense and anti-sense RAP to get the distribution of the expected number of occurrences assuming that the RAPs are randomly distributed on the genome. The 95% quantile of these distributions is 4483 for sense and 8532 for anti-sense RAPs. By comparing these values to the actual number of RAPs containing the motif, it can be shown that there is an over representation of the G₋₁₀Y₋₁G₊₁ motif in the sense inhibitory RAPs.

### Bacterial Strains and Growth Conditions

Two E. coli strains were used: DH5α [F-φ80lacZΔM15 Δ(lacZYA-argF) U169 recA1 endA1 hsdR17 (rₖ−, mₖ+) phoA supE44 λ− thi-1 gyrA96 relA1] and BL21(DE3)pLysS [F−, ompT, hsdSB (rB−, mB−), dcm, gal, λ(DE3), pLysS, Cmr]. Cells were grown in LB media to logarithmic phase (OD₆₀₀=0.5-0.6) or stationary (OD₆₀₀=1.6-1.8) phase with aeration (shaking at 200 rpm) at 37° C. When required, e.g. for plasmid selection, cultures were supplemented with ampicillin (100 μg/ml) or kanamycin (30 μg/ml). Cells with pBAD reporters were induced with 0.2% L-arabinose (Sigma). BL21 cells with pET reporters (transcription by phage T7 RNA-polymerase) were induced with 1 mM IPTG. In experiments with bicyclomycin (BCM) treatment in liquid culture, cultures were first grown to early stationary phase (OD₆₀₀≈0.4). BCM was added to a final concentration of 25 μg/mL or 75 μg/mL (like indicated) and cultivated for 20 minutes before harvesting. In experiments with bicyclomycin (BCM) treatment on plates, cells were grown on LB-agar plates supplemented with BCM added to a final concentration of 8 μg/mL (low concentration allowing cell growth). In growth experiments with stress induction, bacterial cells were cultivated for 20 minutes before harvesting (if not mentioned otherwise).

### Protein Reporter Assays

To monitor expression of RAP-lacZ fusions, E. coli cultures were grown overnight at 37° C. in LB medium with shacking, and afterwards new cultures were started from 1:100 dilution in fresh medium. Cultures were grown to logarithmic phase (OD₆₀₀=0.5-0.6). To determine β-galactosidase activity, corresponding β-galactosidase assays were performed in triplicates as described in (H. Miller, Experiments in molecular genetics (1972); http://books.google.at/books/about/Experiments_in_molecular_genetics.html?id=PtVpAAAAMAAJ&pgis=1).

To monitor GFP fluorescence in transformed E. coli with RAP-GFP fusions, cells were plated by streaking on LB-agar plates supplemented with kanamycin (30 g/ml) and incubated for 16-20 hours at 37° C. Plates were photographed at GFP mode with excitation wavelength 460 nm (Fusion Fx7 Imager, PEQlab, Germany). To compare cell density, plates were imaged at visible light mode with excitation wavelength 510 nm (Fusion Fx7 Imager, PEQlab, Germany).

### RNA Isolation

Total RNA isolation was performed using the hot phenol method. Bacterial cells grown to the particular OD600 (logarithmic phase) were collected and mixed with Stop Solution (95% ethanol, 5% phenol) in 8:1 ratio, followed by centrifugation at 3,000×g at 4° C. for 5 min. The obtained bacterial pellets were frozen in liquid N₂. Pellets were resuspended in a lysis buffer (10 mM Tris-HCl, pH 8.0, 1 mM EDTA, 0.5 mg/mL lysozyme) with the addition of SDS to a final concentration of 0.1%. The obtained lysate was incubated at 64° C. for 2 min, followed by the addition of NaOAc (pH 5.2) to a final conc. of 0.1 M. Then an equal volume of phenol (water-saturated, pH ca 4.0, AppliChem GmbH) was added, gently mixed and incubated at 64° C. for 6 min with inverting 6-8 times. Afterwards, samples were cooled down on ice and centrifuged at 16,100×g (4° C., 10 min). The aqueous phase was mixed with the equal volume of phenol/chloroform/isoamyl alcohol (25:24:1, pH ca 4.0, AppliChem GmbH) and centrifuged for 5 min at 16,100×g (4° C.) in the Phase Lock Gel Heavy tube (5Prime). Then the aqueous phase was mixed with chloroform/isoamyl alcohol (24:1) followed by 5 min centrifugation at 16,100×g (4° C.). RNA was ethanol-precipitated from the aqueous phase (3 volumes ethanol, 1/10 volume of 3 M sodium acetate, 1/100 volume 0.5 M EDTA). To remove traces of DNA, total RNA was treated with Turbo DNase I (Roche) according to the manufacturer's protocol. RNA integrity was checked on agarose gel.

qRT-PCR

To measure the levels of transcripts, 1-2 μg total DNA-free RNA was reverse transcribed using random oligo 9-mers (Sigma) and SuperScript™ II Reverse Transcriptase (Life Technologies) according to the manufacturer's protocol. Depending on the target sequence, cDNA was amplified with the primers described below (see Table 4). In each case the real-time PCR amplification efficiency was estimated using the standard curve method in one color detection system as described before (L. Jolla, 87-112 (2004)). qPCR was performed using Eppendorf Mastercycler® RealPlex2 and 5× HOT FIREPol® EvaGreen® qPCR Mix Plus (no ROX) from Medibena as suggested by the manufacturers. gapA gene levels were used for internal normalization.

### Northern Blot Analysis

For Northern blotting (NB) 10 μg of total RNA was separated by gel electrophoresis using denaturing 8% polyacrylamide-TBE-Urea (8M) gels in 1×TBE. RNA was loaded onto the gel after denaturing at 70° C. (for 10 min) in 2×RNA load dye (Fermentas) followed by transferring on ice. Gel-separated RNA was transferred to HybondXL membranes (Ambion) by wet electro-blotting at 12 V for 1 hour in 0.5×TBE. The membranes were cross-linked by UV (150 mJ/cm²) and probed with 5′-end γ³²P-labeled DNA oligonucleotide probes (see List of sequences) in ULTRAhyb®-Oligo Hybridization Buffer (Ambion) according to the manufacturer's instructions. The DNA labeling reaction was performed with T4 PNK (New England Biolabs) according to the manufacturer's instructions.

### 3′RACE

To precisely determine the 3′end of transcripts, 550 pmol of 5′-phosphorylated RNA adapter (adapter sequence-5′P-AAUGGACUCGUAUCACACCCGACAA-idT (SEQ ID NO:61)) was ligated to 6 μg of total RNA using Ligase 1 for ssRNA (New England Biolabs) according to the manufacturer's protocol overnight at 16° C. The reaction was mixed with the equal volume of phenol/chloroform/isoamyl alcohol (25:24:1, pH ca 4.0, AppliChem GmbH) and centrifuged for 5 min at 16,100×g (4° C.) in the Phase Lock Gel Heavy tube (5Prime). Then the aqueous phase was mixed with chloroform/isoamyl alcohol (24:1) followed by 5 min centrifugation at 16,100×g (4° C.). Nucleic acid was ethanol-precipitated from the aqueous phase (3 volumes ethanol, 1/10 volume of 3 M sodium acetate, 1/100 volume 0.5 M EDTA) and subjected to RT using SuperScript™ II Reverse Transcriptase (Life Technologies) followed by RNaseH treatment (Promega) according to the manufacturer's protocols. cDNA was amplified with Phusion High-Fidelity DNA Polymerase (NEB) using the forward primer to the reporter construct upstream RAP (5′-TTGGGCTAGAATTCGTGTTTA-3′ (SEQ ID NO:62)) and the reverse primer to the ligated adapter (5′-TTGTCGGGTGTGATACGAGTCCATT-3′ (SEQ ID NO:63)). The obtained PCR-amplified library was separated and checked by gel electrophoresis at 6% PAA/1×TBE gel. The obtained bands were gel-purified, TA-cloned using pGEM-T Easy Vector System (Promega, according to the manufacturer's instructions) and subjected to Sanger sequencing. ClustalW2 was used to create the alignments.

### In Vitro Transcription

DNA templates with fused strong T7A1 promoter (SEQ ID NO:58) for in vitro transcription experiments were synthesized by PCR with Phusion Polymerase (New England Biolabs) using reporter plasmids with different RAPs and appropriate synthetic primers (Sigma Aldrich). Transcription reactions were performed in solution. Wild-type E. coli His6 RNAP (Evgeny Nudler Lab) and wild-type E. coli RNAP Holoenzyme (Affymetrix) were used in the assays. To assemble the transcription initiation complexes, 2-4 pmol of His6 RNAP were mixed with 1-2 pmol of DNA template in 20 μl of Transcription Buffer 100 (10 mM MgCl2, 40 mM Tris-HCl, pH 7.9, 100 mM NaCl) (in case of RNAP from Affymetrix, complexes were assembled with same amount of enzyme in 20 mM Tris.HCl pH 8.0 containing 20 mM NaCl, 20 mM MgCl2, 14 mM 2-mercaptoethanol, and 0.1 mM EDTA, as suggested by the company) and incubated for 5 min at 37° C. Then AUC RNA primer (Dharmacon) was added up to 10 μM together with GTP (25 μM) and ATP (25 μM) followed by incubation at 37° C. for 5 minutes. Next, [α-32P]-CTP (800 Ci/mmol, 2 μl, Hartmann Analytic) was added for another 2 min. To minimize RNA degradation 10 U of RNasin® Ribonuclease Inhibitor (Promega) were added to the reaction. For the reactions with Rho and NusG, the purified transcription factors were added in a concentration of up to 0.5 μM to the transcriptional mixtures where indicated, followed by additional incubation at 37° C. for 5 minutes. Chasing reaction was performed with all four NTPs added to a final concentration of 10-50 μM. To prevent re-initiation, rifampicin (Sigma) was added concurrently at this step to a final concentration of 10 μM. At the indicated time points, an aliquot of chase reaction was quenched with 3× Stop Solution, containing 100 mM EDTA, 8M UREA, 0.025% xylenthianol and 0.025% bromophenol blue. The products were separated by 6% denaturing TBE-PAGE (8M urea). RAP sequence mapping within the transcript was performed by transcribing RAP-containing template and 25 μM of NTPs mixed with one of the four 3′ dNTPs (3′dGTP, 3′dATP, 3′dUTP or 3′dCTP) at 3:1 ratio in four different transcription reactions for 10 min.

Construction of Reporter Construct pWM3110

The assays performed herein use a reporter system based on the plasmid pWM3110 as constructed by the present inventors. This construct was obtained by adapting a well-characterized GFP-containing plasmid pWM015 (W. G. Miller et al., Appl. Environ. Microbiol. 66, 5426-36 (2000), and C. H. Eggers et al., Mol. Microbiol. 43, 281-95 (2002), both incorporated herein by reference). pWM015 contains a Kanamycin resistance gene (Kan^{R}) and the gene encoding for the green fluorescence protein (GFP) under control of a bacterial constitutive promoter (SEQ ID NO:66). pWM1015 was adapted through extending 5′UTR of the GFP gene with additional 108 nt in order to introduce unique XhoI and SacI restriction enzyme recognition sites for successful cloning in the 5′UTR. For this purpose, the insert of SEQ ID NO:64 was cloned between EcoRI site in the original pWM015 plasmid. pWM015 was digested (linearized) using EcoRI (Thermo Scientific) according to manufacturer's protocol. The synthetically produced double stranded oligonucleotide of SEQ ID NO:64 (Sigma Aldrich) was digested using EcoRI and ApoI (Thermo Scientific) according to the manufacturer's protocol, resulting in overhangs compatible with EcoRI overhangs to allow proper orientation of the construct. The digestion products were ligated with T4 DNA Ligase (New England Biolabs) according to the manufacturer's protocol. Correct and single insertion was confirmed by bacterial colony PCR and subsequent Sanger sequencing. The sequence of the resulting pWM3110 is given as SEQ ID NO:65.

The different sequences encoding RNA-Polymerase binding aptamers were cloned into pWM3310, supra, using the unique XhoI and SacI restriction sites. The synthetically produced (Sigma Aldrich) double stranded SEQ ID NO:68 (containing rut Rho termination insert), SEQ ID NO:69 (containing rrnB intrinsic terminator insert), SEQ ID NO:70 (containing T7t intrinsic terminator insert), or SEQ ID NO:71 (containing the combination of three T7t-tR2-rrnB intrinsic terminators) were digested with SacI (site at the 5′ end of each construct) and BsiHKA1(ALw211) (a SacI compatible restriction site on the 3′end allowing preservation of SacI as a unique restriction site in the final construct) according the manufacturer's protocol. Thereafter, the digested and purified constructs were cloned into SacI-linearized pWM3310 by ligation with T4 DNA Ligase (New England Biolabs) according to the manufacturer's protocol. Correct and single insertion was confirmed by bacterial colony PCR and subsequent Sanger sequencing. The constructs were named according to the terminator sequence they contained as pWM3110_rut, pWM3110_rrnB, pWM3110_T7t, and pWM3110_triple terminator, respectively.

For assays employing lacZ as the reporter gene a derivative of pBAD24 was constructed. pBAD24 contains an bacterial arabinose-inducible promoter (SEQ ID NO:67) and an ampicillin resistance gene (Amp^{R}) (L. M. Guzman et al., 177 (1995), incorporated herein by reference). The original ribosomal binding site of pBAD24 was removed from the multiple cloning site by restriction with NheI/EcoRI, followed by DNA Polymerase I Large (Klenow) Fragment treatment and blunt ligation (recirculation) to obtain pBAD13. A new +70nt RBS (SEQ ID NO:80) was cloned between the XbaI/PstI restriction sites and the lacZ gene (SEQ ID NO:79) between the PstI/HindIII restriction sites of pBAD13 to obtain pBAD13_lacZ.

The different RNA-polymerase binding aptamers were cloned into pBAD13_lacZ between the EcoRI/NcoI restriction sites to obtain pB13_RAP_lacZ. To ensure that the observed effects are not affected by the initial RAP 3′end-flanking sequence a further +99nt insert (cobC-derived, no RAP contained SEQ ID NO:80) was inserted through NcoI/Xbal restriction sites. The resulting plasmid was pB13_RAP_+99_lacZ. To ensure that the effects observed were also not affected by initial RAP 5′-flanking sequences the respective RAP sequence was exchanged by a construct containing additional 49 nt (SEQ ID NO:81) at the 5′-end of the RAP and the RAP-insert using the EcoRI/NcoI restriction sites, resulting in pB13_+49RAP_+99_lacZ.

A further construct was created to test effects on expression from T7 promoter. To this end, the orginal RBS was removed from the MCS of pET21a (Novagen) by Xba/NdeI restriction, followed by DNA Polymerase I Large (Klenow) Fragment treatment and blunt ligation (recirculation). The resulting construct was named pET21_minRBS. The RAPs were cloned into pET21_minRBS by amplification of the full length RAP_+99_lacZ including additional 5 nt from the transcription starting site from pB13_RAP_+99_lacZ as the template and pET21_Insert_FW_NheI (SEQ ID NO:82) and pET21_Insert_RV_HindII: (SEQ ID NO:83) as primers. The amplification product was cloned into pET21_minRBS using the NheI/HindIII restriction sites.

### Example 1: Transcription Enhancing RNA-Polymerase Binding Aptamers

Identification of RAPs that Promote Activation of Transcription

To monitor the potential effect of identified RAPs on transcription, they were tested using a plasmid-based GFP reporter pWM3110 (see FIG. 2A; and supra). Several representative RAPs were cloned into the 5′UTR of the reporter construct (distance downstream of TSS—72 nt, distance upstream of GFP's RBS—56 nt). The obtained clones were tested in the E. coli GFP plate assay to estimate the amount of fluorescent protein synthesized in each case. By this approach a number of RAPs could be identified as listed in Table 1—e.g., RAP ID #5713 (SEQ ID NO:2) and #14908 (SEQ ID NO:4)—with a novel mode of activity: they significantly increased the amount of GFP produced (FIG. 2B). The inventors also performed qRT-PCR to directly measure the amount of full-length GFP transcript, confirming the results of the protein assay: for example, RAPs #5713 and #14908 increase transcriptional levels more than 2 and 1.5 times, respectively (FIG. 2C). We categorized RAPs that increased GFP transcription/expression for at least 20% (in comparison to no RAP control) as activating ones. Corresponding sequences are presented in Table 1.

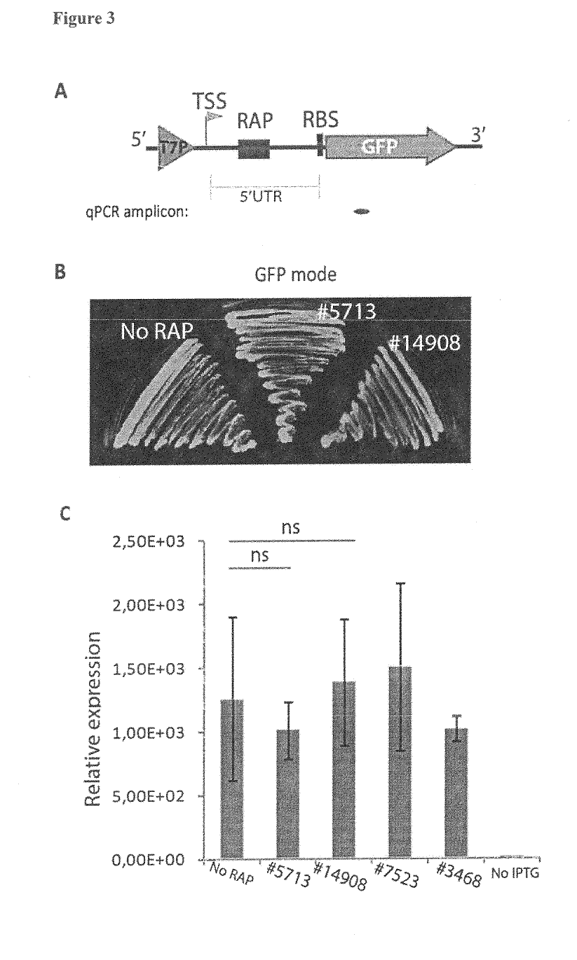

To test for specificity, the same RAP-containing reporter constructs were expressed from the phage T7 promoter using phage T7 RNA Polymerase (FIG. 3A). In this case no up-regulation could be detected for both at protein (FIG. 3B) nor at RNA (FIG. 3C) levels. As there is no difference in expression levels compared to no RAP control, we conclude that activating activity of RAPs is specific for bacterial RNA polymerase and not for the distant RNAP from phages.

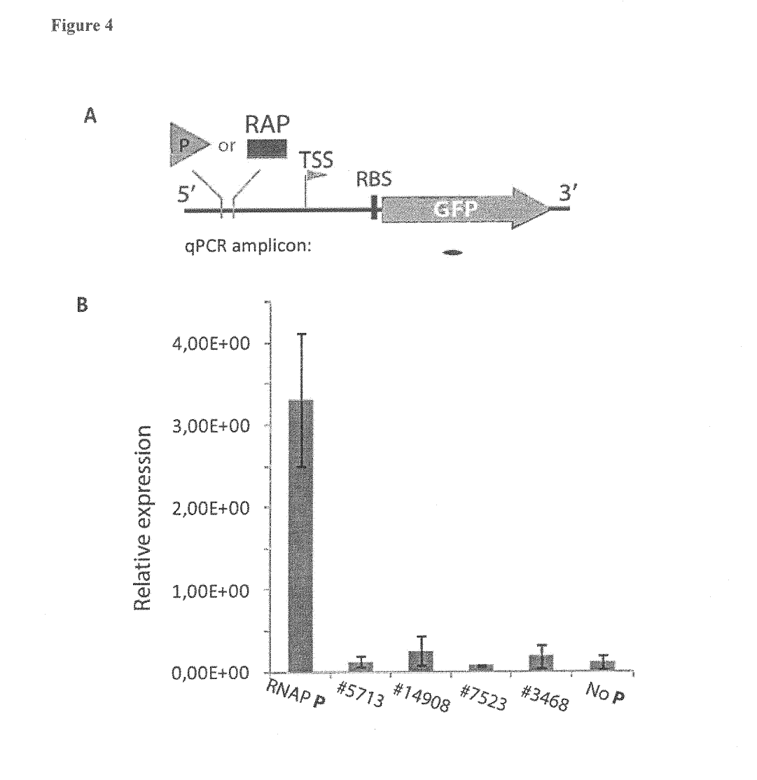

Control experiments were performed to check if the observed transcriptional activation could be explained by enhanced promoter-like RNAP recruitment to the RAP-encoding DNA sequence. To rule it out, activating RAPs were checked in a promoter activity assay. RAPs were cloned individually to the reporter construct replacing the constitutive promoter (FIG. 4A). The efficiency of transcription from these constructs was estimated by qRT-PCR. The results of these measurements (FIG. 4B) suggested that activating RAPs do not exhibit promoter activity.

Further checked was whether activating RAP#5713 would activate transcription when being located upstream of the promoter, in a non-transcribed region (FIG. 5A). No difference in transcription levels was observed by qRT-PCR (FIG. 5B). Taken together, the data show that RNA-polymerase binding aptamers according to the present invention can upregulate transcription when being transcribed to RNA from the upstream promoter.

### Identification of RAPs Promoting Antitermination of Transcription

For further characterization activating RAPs capacity to suppress termination was investigated. Two types of termination signals have been described in bacteria: factor-independent (intrinsic) and factor-dependent (Rho-dependent) ones (Santangelo and Artsimovitch, 2011).

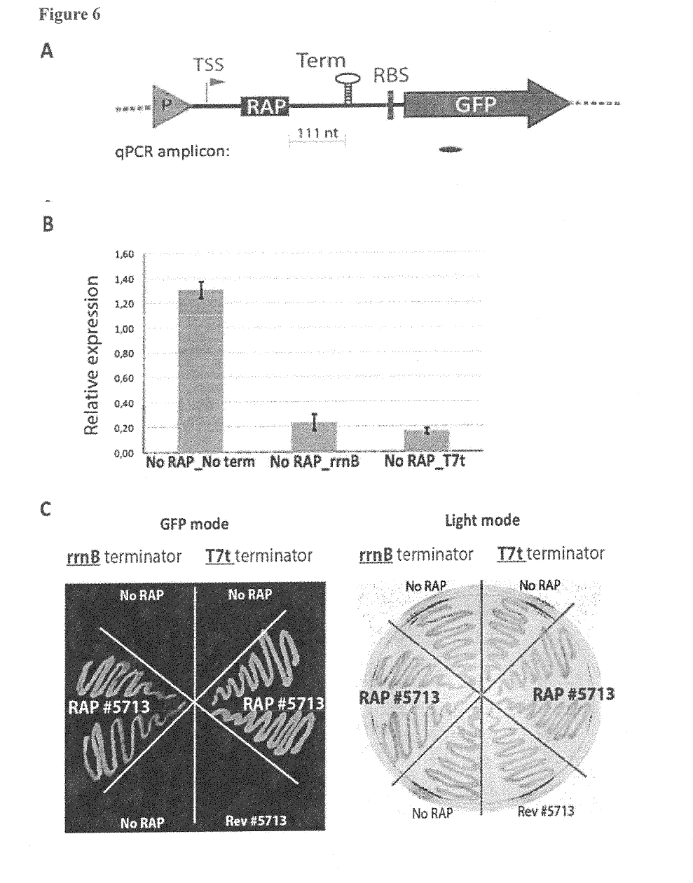

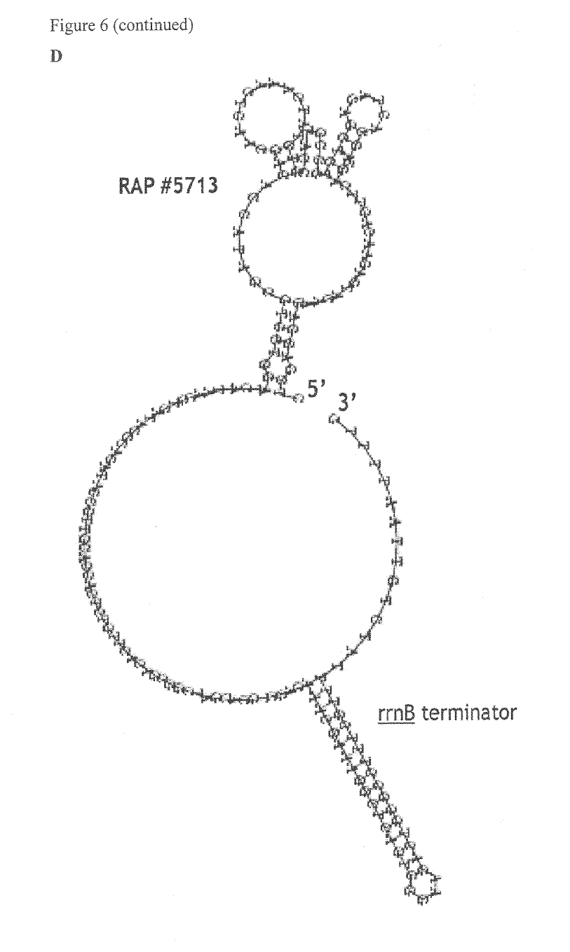

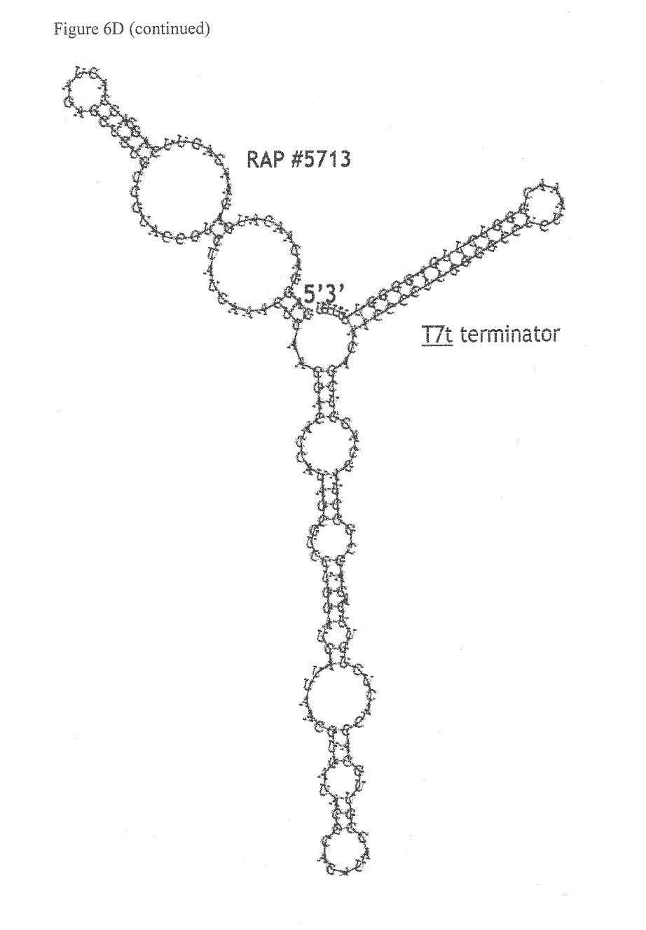

First, the effect of activating RAPs on the efficiency of read-through the intrinsic termination signal was tested. Two different heterologous RNAP intrinsic terminators were cloned in-between a RAP signal and the GFP reporter (FIG. 6A). In the absence of RAP, the selected well-characterized intrinsic terminators rrnB and T7t reduced the levels of GFP transcripts to 18% and 13%, respectively, when compared to no intrinsic terminator (FIG. 6B). Surprisingly, tested activating RAPs strongly increased read-through the terminators. As seen in FIG. 6C, in the absence of RAP #5713, GFP expression is low due the presence of the terminators (rrnB or T7t terminators), but as soon as a RAP #5713 is present upstream of the terminator hairpin, the RNA polymerase no longer efficiently terminates. The reverse complement of RAP #5713 (marked as Rev #5713) had no activation/antitermination effect on transcription, it was used as control sequence. Precise quantification by qRT-PCR confirmed that RAP #5713 act as an antiterminator, increasing the efficiency of read-through the terminator by a factor of 8-14 when compared to no RAP control (FIG. 6E-6F). Importantly, RAP #5713 sequence does not interfere with folding of the terminator hairpins (FIG. 6D).

RAPs that Suppress Rho-Dependent Termination

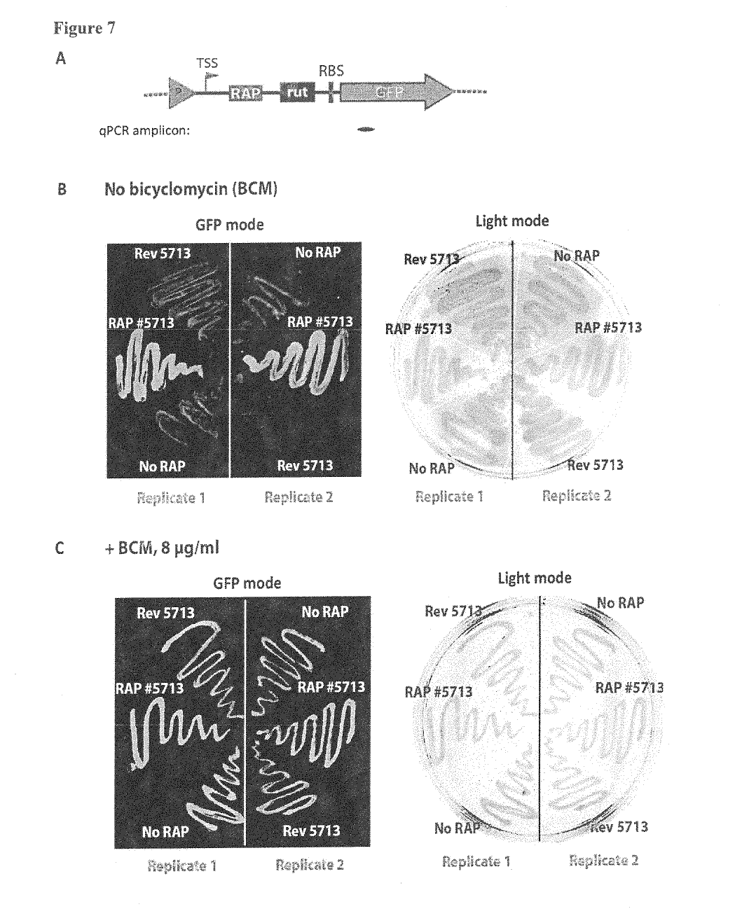

The activating RAPs where tested as signals able to activate read-through the Rho-dependent termination signal. Rho-dependent termination requires Rho binding to specific nascent RNA regions, named Rho utilization (rut) sites. One of the well-characterized rut sites (Krebs et al., “Lewin's GENES X”, 2009) was inserted in between RAP #5713 and the reporter gene GFP (FIG. 7A). In the absence of RAP #5713, reporter GFP is expressed at low levels. However, insertion of RAP #5713 upstream the rut site results in increased GFP expression (FIG. 7B). These data were further confirmed by measuring transcripts levels by qRT-PCR (data not shown). Additionally, to control for Rho-dependence, bicyclomycin (BCM) was used—a highly specific antibiotic that inhibits Rho (Zwiefka et al., 1993). In the presence of BCM, GFP is efficiently expressed from the reporter without RAP; in the presence of BCM RAP#5713 can no longer activate transcription and expression levels are unchanged (FIG. 7C). This clearly demonstrates that RAP#5713 interferes with Rho-dependent termination.

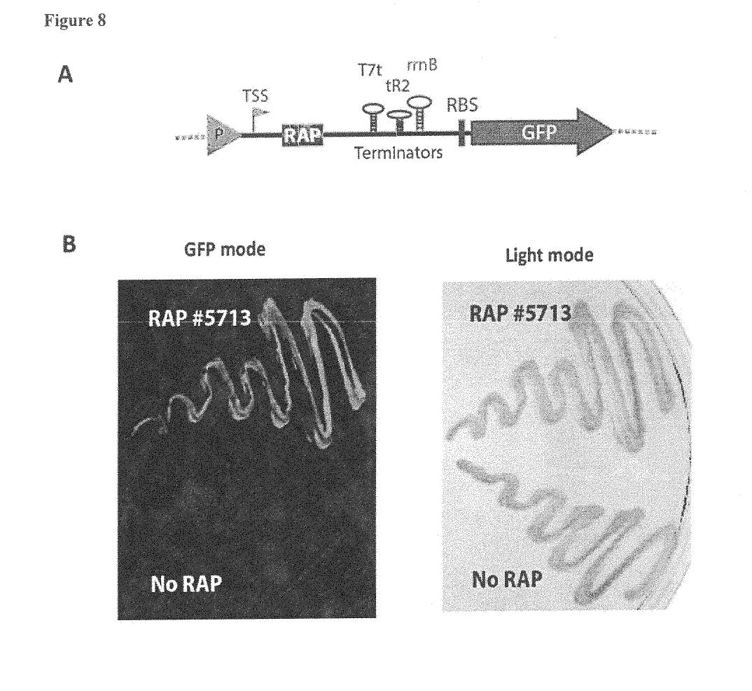

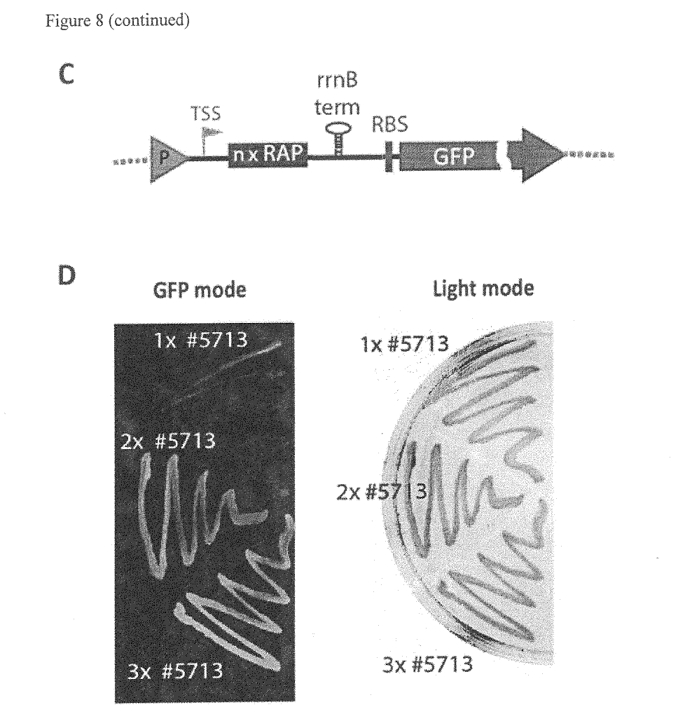

The antitermination potential of RAP #5713 (SEQ ID NO:2) was further analyses by inserting 3 heterologous intrinsic terminators in a row upstream of GFP (FIG. 8A; see SEQ ID NO:45). As in the previous experiments, supra, presence of RAP #5713 resulted in high expression levels of GFP when compared to no RAP control (FIG. 8B). Insertion of several RAP #5713 sequences in tandem (FIG. 8C) results in the liner increase of antiterminatory activity, resulting in higher GFP expression (FIG. 8D). It makes use of the activating/antiterminating RAPs especially appealing for the biotechnology sector. This clearly shows that the mode of action of the RAPs is independent on the specific terminator.

### Demonstration of RAP Antitermination Activity Through In Vitro Transcription Assays

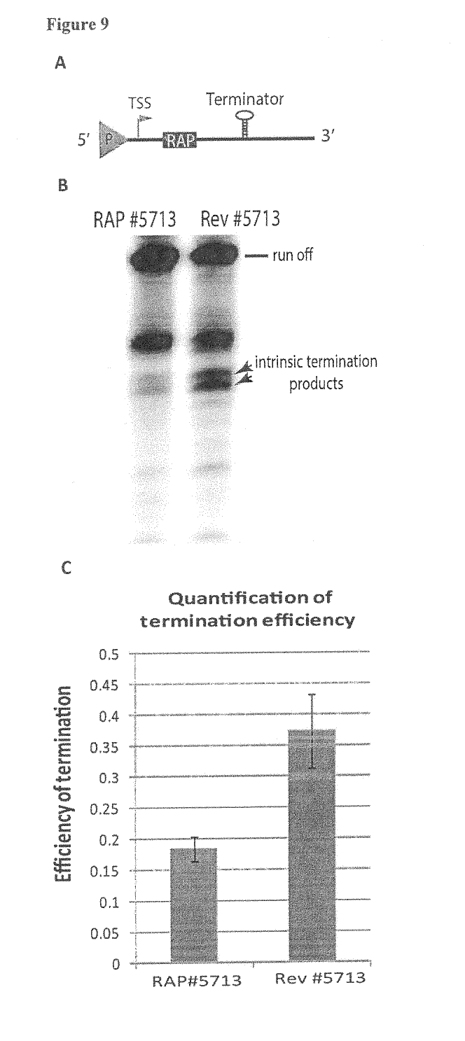

To gain a first insight into the mechanism of RAP-mediated transcription antitermination, in vitro transcription studies were performed to test if direct RAP#5713-RNAP interaction would be sufficient for efficient antitermination and transcription enhancing, respectively. Template with intrinsic terminator rrnB was used for these studies. RAP#5713 or control Rev#5713 (sequence, reverse complement to RAP#5713) was placed upstream of the terminator (FIG. 9A). Transcription was performed with purified E. coli RNAP only, without any additional transcriptional factors. Single round transcription from RAP#5713-containing template resulted in lower amount of intrinsic termination products and higher levels of run-off products when compared to the control construct (FIG. 9B). Precise quantification revealed that presence of RAP#5713 reduced termination activity of RNAP more than 2 times, resulting in higher accumulation of full-length run off product (FIG. 9C). These data demonstrate that antitermination activity of RAP#5713 is promoted by direct interaction of RAP and RNAP.

### Large Scale Screen for Activating RAPs

To gain insight into the frequency of activating RAPs in the 7^{th} cycle SELEX pool, they were cloned into the reporter construct shown in FIG. 2A. From this in-depth screening of total RAP pool the transcription enhancing RAPs as summarized in Table 1 were identified as enhancing transcription when cloned into the reporter construct. Taken together, the presented data clearly show the high transcription enhancing potential of RAPs, resulting in highly efficient read through all types of bacterial RNAP termination signals and elevated protein expression.

### Structural and Mutational Analysis of the Identified Transcription Enhancing RAPs

To identify further common features of the transcription enhancing RAPs sequence and secondary structure analysis were performed.

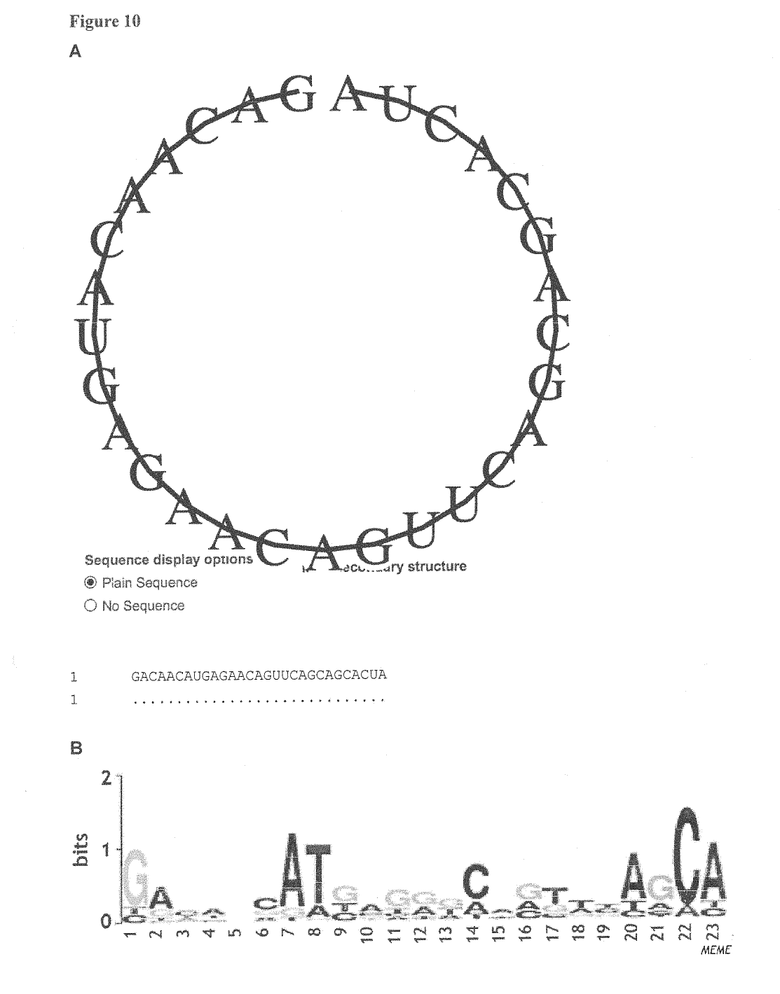

In contrast to the known antiterminatory phage sequences, the transcription enhancing RNA-polymerase binding aptamers according to the present invention are (a) much shorter, (b) have no common particular requirements for complex secondary structure according to predictions with specialized software (like Vienna RNA Package, RNAfold—http://rna.tbi.univie.ac.at/cgi-bin/RNAfold.cgi; or mfold http://mfold.rna.albany.edu/?q=mfold). An analysis of the secondary structure of RAP ID #5713 (SEQ ID NO:2) is shown in FIG. 10A.

Sequence analysis has been performed with the sequences of SEQ ID NOs:2 to 14 as well as the sequences of mutated variants of RAP ID #5713 (SEQ ID NO:72-75) to identify a common motif. All sequences were combined with the RAP ID #5713 (SEQ ID NO:2). Prediction was performed with The MEME Suite 4.10.0: Motif-based sequence analysis tools (T. Bailey, M. Bodén, F. Buske, M. Frith, C. Charles E. Grant, L. Clementi, J. Ren, W. Li, W. Noble, “MEME SUITE: tools for motif discovery and searching”, Nucleic Acids Research, 37:W202-W208, 2009). Reported activating RAP sequences were submitted to MEME program with the following parameters: “site distribution”—one per sequence; “search given strand only”. The analysis revealed a consensus sequence of SEQ ID NO:1 for enhancing RAPs (see FIG. 10B; and Table 7). Table 7 visualizes the position of the consensus sequence within the different identified enhancing RAPs and mutant variants thereof as well as the p-value.

### Absence of RAP#5713 Activity in Trans

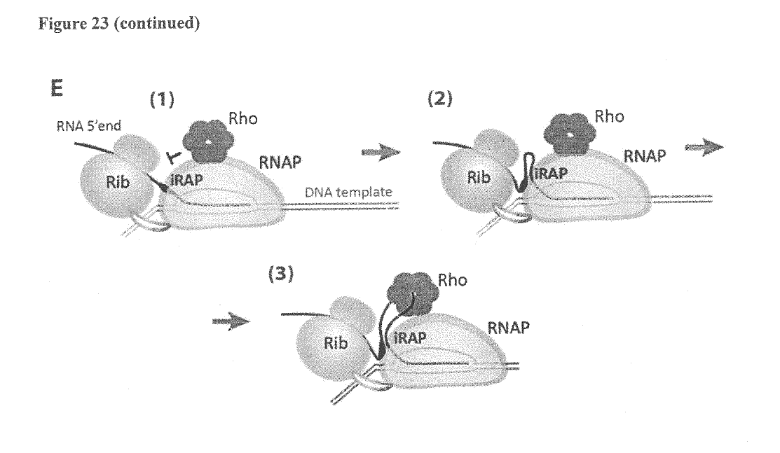

To test for potential activity of the named activating RAPs in trans in vivo, a reporter system was designed consisting of two compatible plasmids suitable for the co-transformation in E. coli (FIG. 21A). The first plasmid contained GFP reporter with a well-characterized transcription terminator (intrinsic (rut) or Rho-factor-dependent (rrnB) between transcription starting site and ribosomal binding site upstream of the GFP-ORF. The second plasmid was designed for arabinose-induced overexpression of RAP#5713 as a representative example in form of a stable sRNA. Three different RAP_sRNAs (RAP_sRNA1-RAP_sRNA3) were constructed by using a DNA encoding the RNA scaffold of DsrA, RprA and their hybrid sRNA, respectively and replacing the sequence coding for base-pairing region with the corresponding DNA sequence coding for RAP#5713 such that the overall folding was not affected. The overexpression and accumulation of stable RAP_sRNAs were confirmed by Northern blot analysis on the total RNA from L-arabinose-induced transformed host cells. pBAD30 plasmid without sRNA was used as a control (FIG. 21B). Interestingly, although RAP#5713 was placed right upstream of several terminators in this case, it still allowed some read-through with RNAP (see FIG. 21B. smear upstream of the major band). The potential antitermination effect of three RAP_sRNAs overexpressed in trans was tested in the system with both, intrinsic (FIG. 21C) and Rho-dependent (FIG. 21D) terminators, upstream of the reporter GFP. No difference in GFP intensities was observed between the transformed strains, showing that RAP#5713 is not active in trans. The growth of strains with overexpressed RAP_sRNAs was also not affected—neither on the plate (FIG. 21C-D), nor in liquid media in all phases (data not shown), also supporting the hypothesis that RAP#5713 does not affect transcription when acting in trans.

The presented results provide an essential advantage for the targeted use of activating RAPs as, while activating the expression of the desired sequence in cis, they do not affect the expression of the other host genes in trans.

RAPs with Transcription Enhancing Properties Derived from Eukaryotic Organisms

By the definition, RAPs are natural RNA aptamers binding RNAP with high affinity. The above RAPs originate from the bacterial (E. coli) genome. Further investigations were preformed using genomic SELEX with the RNAP II for yeast and Homo sapiens. These yeast/human RAP sequences with high affinity to the eukaryotic RNAP have been named yh1 to yh45 (SEQ ID NO:93 to 137). Taking into account the conservation of RNAP machineries between all the cellular organisms, it follows that the obtained yeast/human RAPs can activate and antiterminate transcription by prokaryotic RNAP in cis, possessing the corresponding activities as previously above bacteria derived RAPs. To prove this, a number of eukaryotic RAPs was tested for the effect on transcription activation in bacteria. The pWM3110_rrnB construct with GFP reporter (schematically presented in FIG. 6A) was used for fast and efficient test of human/yeast RAPs activity on bacterial transcription. As a result, the non-bacterial sequences (non-bacterial RAPs) have been shown to increase transcription output in E. coli, presumably via the RAP RNA—bacterial RNAP interactions (similar to the mechanism utilized by the bacterial RAPs).

The presented sequences can be described with a Regular Expression for Motif: [AC][AC][AG]CAN[AC][AT][CTG]A[CT][AT]CCNA[AC]N[AT]C[AC] (SEQ ID NO:138; prediction with GLAM2, see FIG. 3).

Further, a motif common for all enhancing aptamers, pro- and eukaryotic has been identified using GLAM2 prediction which is reflected in SEQ DI NO:139. As will be acknowledged by the skilled person, this motif also very well reflects the essential nucleotides of the core motif as identified for the RAP encoded by SEQ ID NO:2.

### Example 2: Transcription Inhibiting RNA-Polymerase Binding Aptamers

Identification of RAPs that Inhibit Transcription

The enriched sequences obtained under the most stringent selection conditions (7 cycles of genomic SELEX) were deep-sequenced and ˜1.0 million reads were mapped to the E. coli K12 MG1655 genome followed by peak calling using a custom algorithm. Overall, we identified over 15,000 RAPs—RNA aptamers with high affinity to RNAP—in the E. coli genome (Table S1). The Kd of the total selected pool was shown to be below 10 nM (see N. Windbichler, F. von Pelchrzim, O. Mayer, E. Csaszar, R. Schroeder, RNA Biol. 5, 30-40). The majority of RAPs (64.3%) maps antisense to genes and approximately ⅓ of them (31.5%) are intragenic (FIG. 1, A-B). The positive control, 6S RNA (see K. M. Wassarman, G. Storz, Cell. 101, 613-23 (2000); and A. T. Cavanagh, K. M. Wassarman, Annu. Rev. Microbiol. (2014), doi:10.1146/annurev-micro-092611-150135), was detected within the identified RAPs (RAP #10012, Table S1), validating the strategy for the genome-wide search of RNAP-binding RNAs. Interestingly, RAPs are overrepresented in the proximity of translation start sites and underrepresented in intergenic regions. Antisense RAPs enrichment opposite to the 3′-proximal part of ORFs was observed (FIG. 1, C-D). Different tools (MEME and Homer) were used to search for common sequence motifs within RAPs and could only identify a CA-rich motif in 12% of the peaks (FIG. 14A). From these results we conclude that RNAP can bind a large variety of RNA molecules.

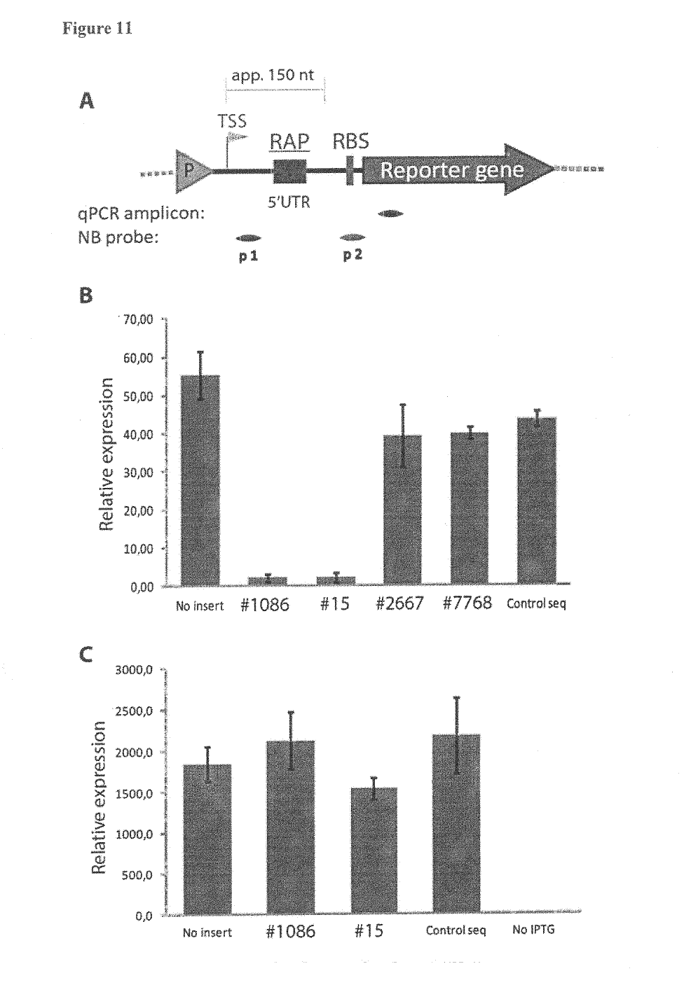

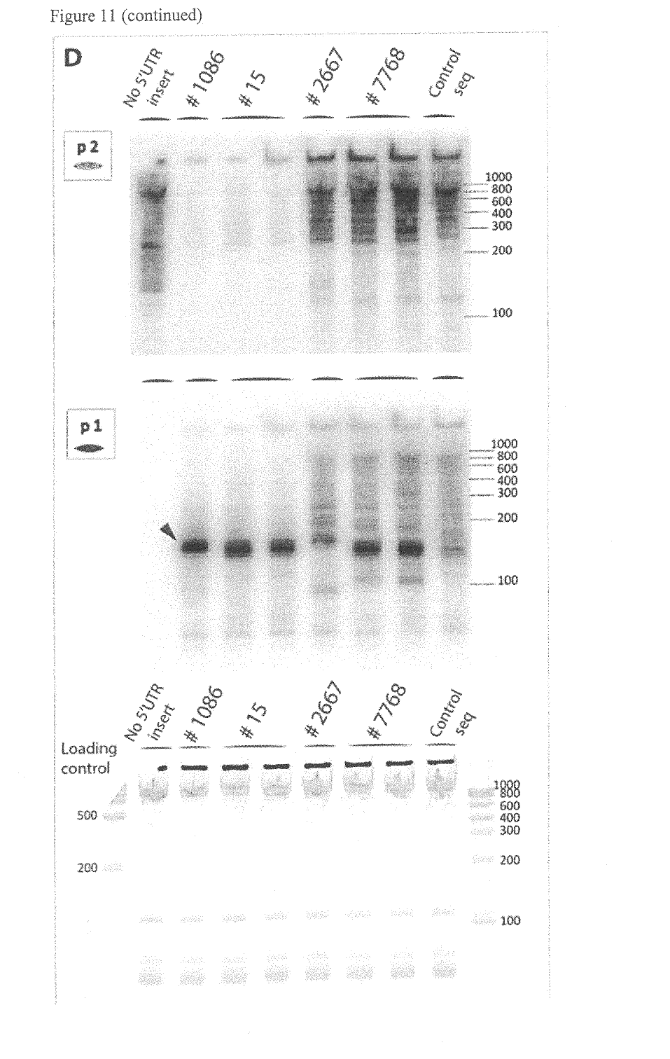

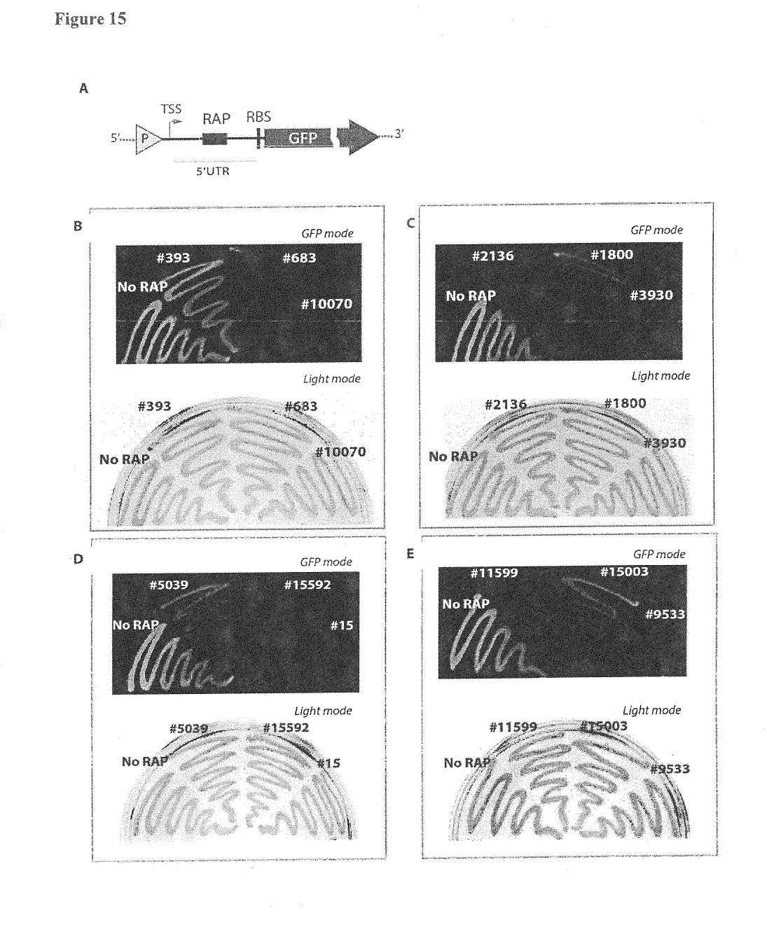

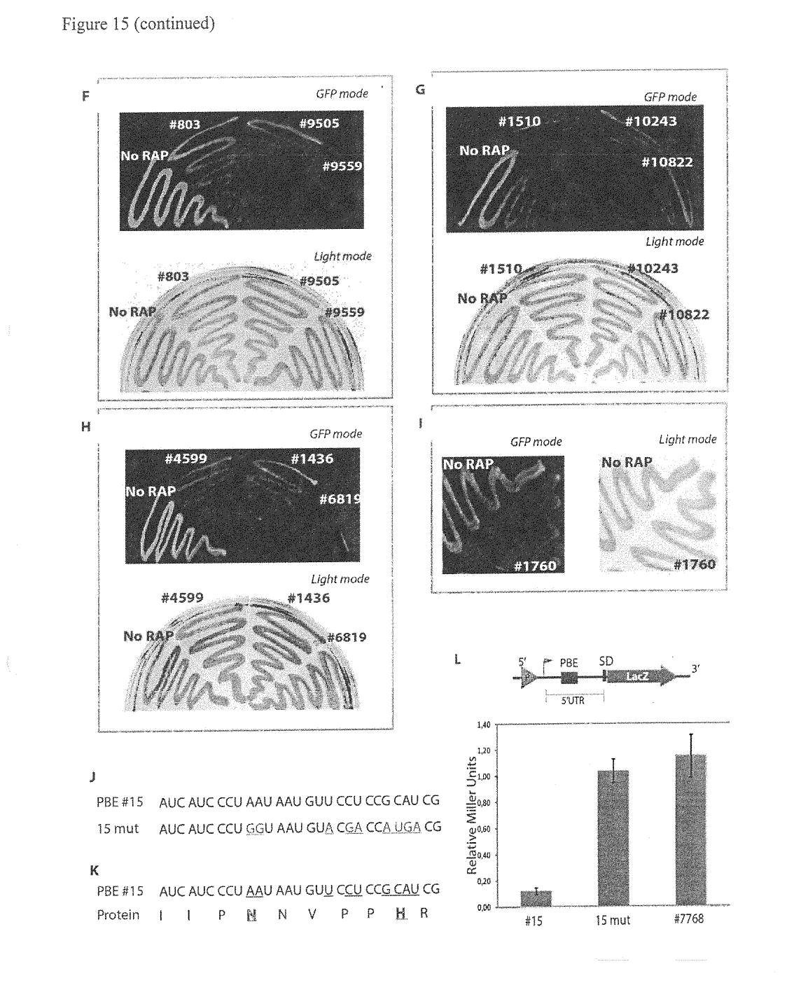

To elucidate a possible effect of RAPs on transcription, the activity of representative sense and antisense RAPs was tested using a plasmid-based LacZ reporter (FIG. 11A). The influence of RAPs on gene expression was evaluated by monitoring changes in LacZ activity and RNA levels by quantitative RT-PCR (qRT-PCR). Several RAPs could be identified that strongly reduce the transcriptional output; for example, the antisense RAP #1086 and intragenic RAP #15 caused major reduction in transcript levels (FIG. 11B). To ensure that the observed inhibitory effect was not promoter- or sequence context-dependent, these RAPs were tested in an additional GFP reporter system derived from a different plasmid (FIG. 15, A-I). To estimate the overall fraction of RAPs with transcription inhibitory properties we examined 76 RAPs and found 20 (˜26%) to exhibit this activity. SEQ ID NOs:25 to 41 of Table 1; infra; give an overview of these inhibitory RAPs indicating their genetic location, category, sequence and the number of mapped bases from deep sequencing. More than half of these inhibiting RAPs are intragenic, representing RNA domains within protein-coding genes. Upon extrapolation, obtained numbers suggest that one-fourth of all identified RAPs might have transcription suppressing activity.

To test the specificity of the observed RAP effect for bacterial RNAP, the “inhibiting” RAPs were cloned in a similar reporter system with the phage T7 promoter. IPTG-inducible E. coli BL21 strain was used to ensure transcription by phage T7 RNAP. In this case, we did not observe any significant reduction of transcript levels in response to RAPs (FIG. 2C). Therefore, the inhibitory effect of RAPs on gene expression is specific for bacterial RNAP and not due to reduced RNA stability.

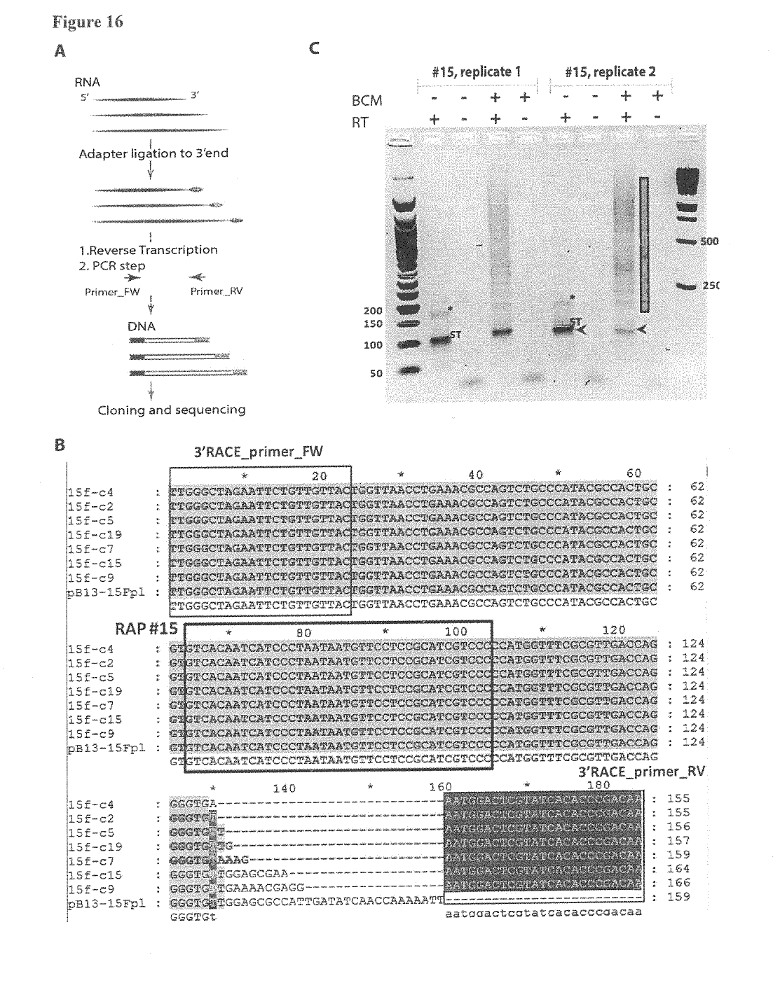

To gain further insight into the mechanism leading to transcription inhibition by RAPs, Northern blot analysis of RAP-containing transcripts were performed. Total RNA from logarithmic growing cells was probed with oligonucleotides annealing before and after the RAPs (probe location is indicated in FIG. 11A). Hybridization with the downstream probe P2 (FIG. 2D) confirmed our RT-qPCR data, i.e. virtually no RNA was detected in the case of the inhibitory RAPs (#15 and #1086). However, hybridization with the upstream probe P1 revealed the accumulation of stable truncated RNA products (FIG. 11D). As judged by size, these transcripts were terminated approximately 30 nt downstream of the inserted RAPs. The fuzzy shape of the corresponding bands also suggested the absence of a precise termination point. These data were complemented with 3′RACE experiments mapping the exact termination sites (FIG. 16, A-B): the prevailing transcripts contained the complete RAP sequence and had the 3′ ends mapped 27 to 38 nucleotides downstream of the RAP. To confirm that the inhibitory effect was due to the RAP sequence, several point mutations were introduced into RAP #15 (FIG. 15, D-E), which abolished its inhibitory effect on transcription (FIG. 15, F).

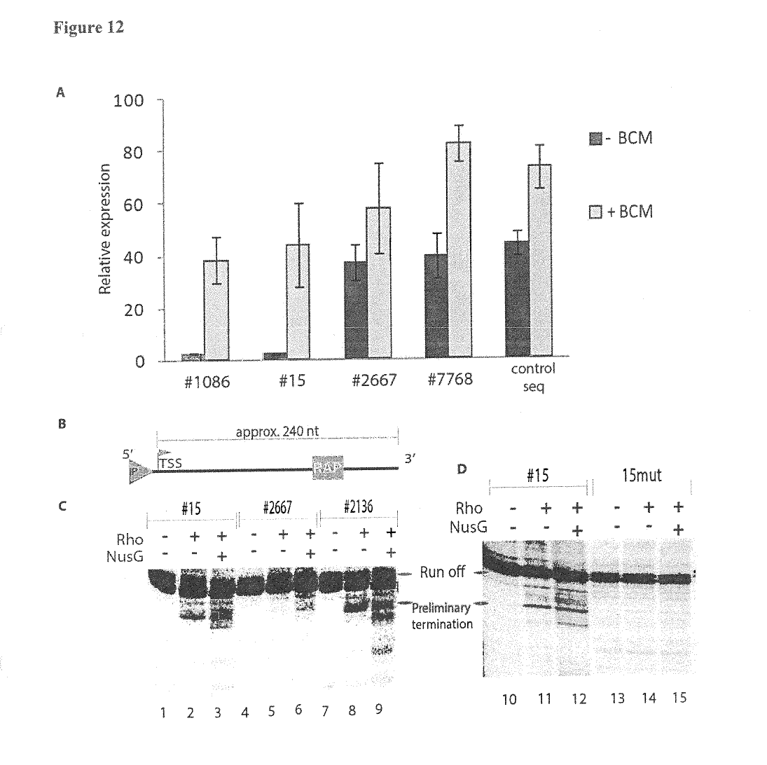

To elucidate the mechanism of RAP-triggered transcription termination, it was searched for possible terminator hairpins formed between the RAP and downstream sequences in the reporter construct. No potential intrinsic terminators could be identified. Hence, RAP-mediated termination may be Rho-dependent. To test this, bicyclomycin (BCM)—a highly specific antibiotic that inhibits Rho (14)—was utilized. Upon addition of BCM (25 μg/ml) to the cells transformed with the RAP constructs described above, transcription inhibition by RAPs #1086 and #15 was abolished (FIG. 12A). Transcription through RAPs #2667 and #7768, which had no inhibitory effect, was much less affected by BCM. This indicates that RAP-mediated premature termination of transcription is Rho-dependent. These data were also complemented by 3′RACE experiments in the presence of BCM showing the accumulation of full-length RAP-containing transcripts (FIG. 16, C).

To directly confirm Rho dependence of the RAP effects, single round in vitro transcription assays using synthetic DNA templates with selected RAPs and the strong T7A1 promoter were performed. Without Rho, the in vitro transcription with E. coli RNAP yielded full-length runoff transcripts (FIG. 12B, lanes 1, 4, 7). The addition of Rho resulted in the formation of shorter RAP-containing transcripts in the case of inhibitory RAPs (e.g., #15 and #2136—FIG. 12B, lanes 2, 8 vs. lane 5). The termination products were enhanced in the presence of NusG, a cofactor known to increase the efficiency of Rho-dependent termination (FIG. 12B, lane 3, 9). In vitro transcription experiments showed that the addition of Rho factor to the reaction with the mutated RAP #15 (15 mut) template did not lead to Rho-dependent termination (FIG. 12C). These data demonstrate that the inhibitory effect of RAP #15 is due to Rho-dependent termination.

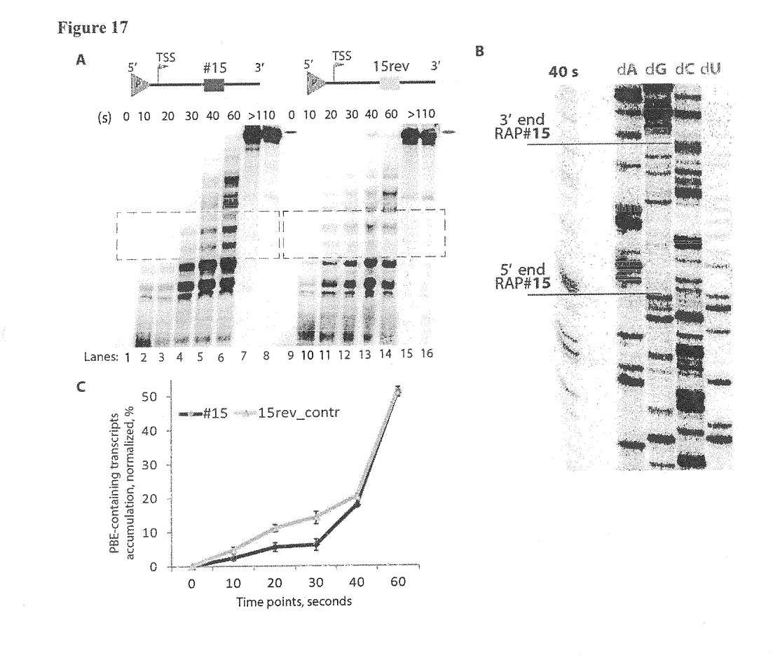

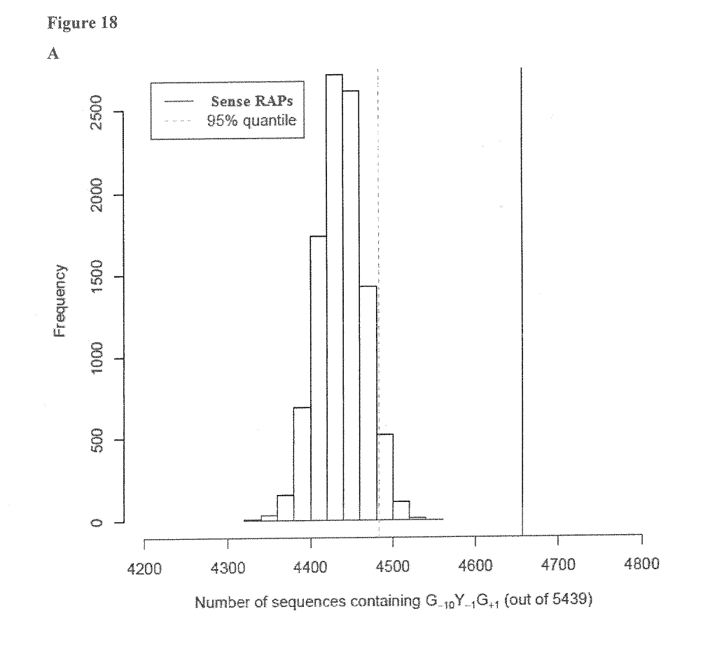

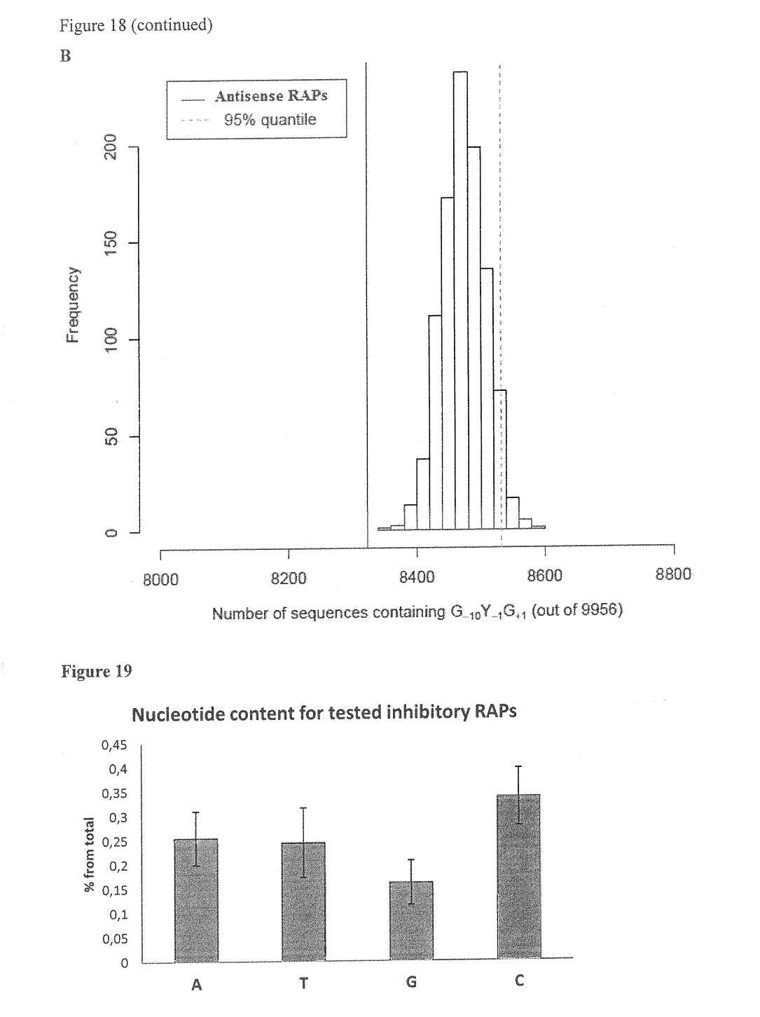

To investigate whether RAPs affected elongation by E. coli RNAP, single-round time-course transcriptional assays using DNA templates with RAP #15 and its reverse complement sequence at the same position as a control were performed. The position of RAP was precisely mapped (FIG. 17B). We detected strong RNAP pausing within the RAP#15 sequence and several nucleotides downstream (FIG. 17A). We quantified the observed effect as the ratio of full RAP-containing transcript amount to the transcripts that had not incorporated the RAP sequences for each time point. The accumulation of transcripts containing the whole RAP sequence was at least two fold less efficient in case of RAP#15 in comparison to the control sequence (FIG. 17C). This experiment shows that RAP #15 induces transcription pausing. Interestingly, the G₋₁₀Y₋₁G₊₁ motif, recently identified with NET-seq approach and reported to be a consensus for pausing (M. H. Larson et al., Science. 344, 1042-7 (2014); and I. O. Vvedenskaya et al., Science. 344, 1285-9 (2014)), is overrepresented in the subgroup of sense RAPs (FIG. 18, A-B).

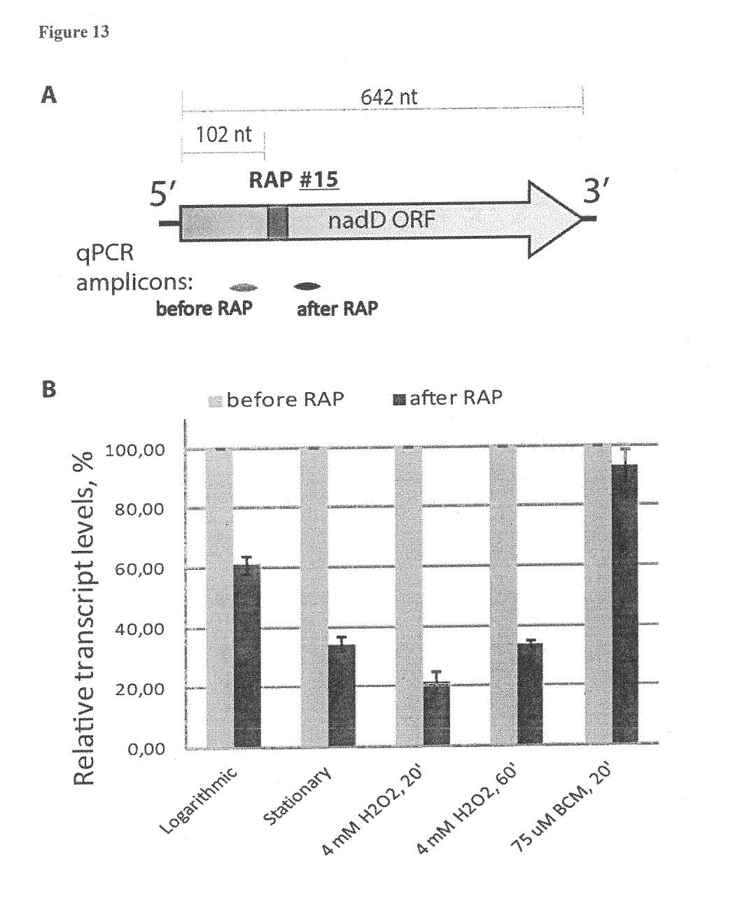

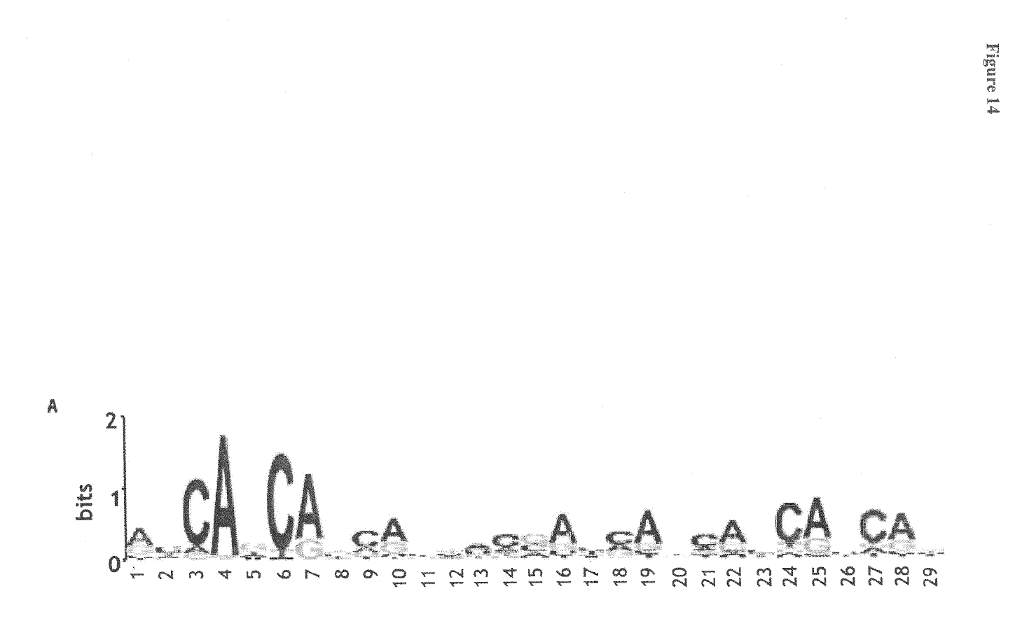

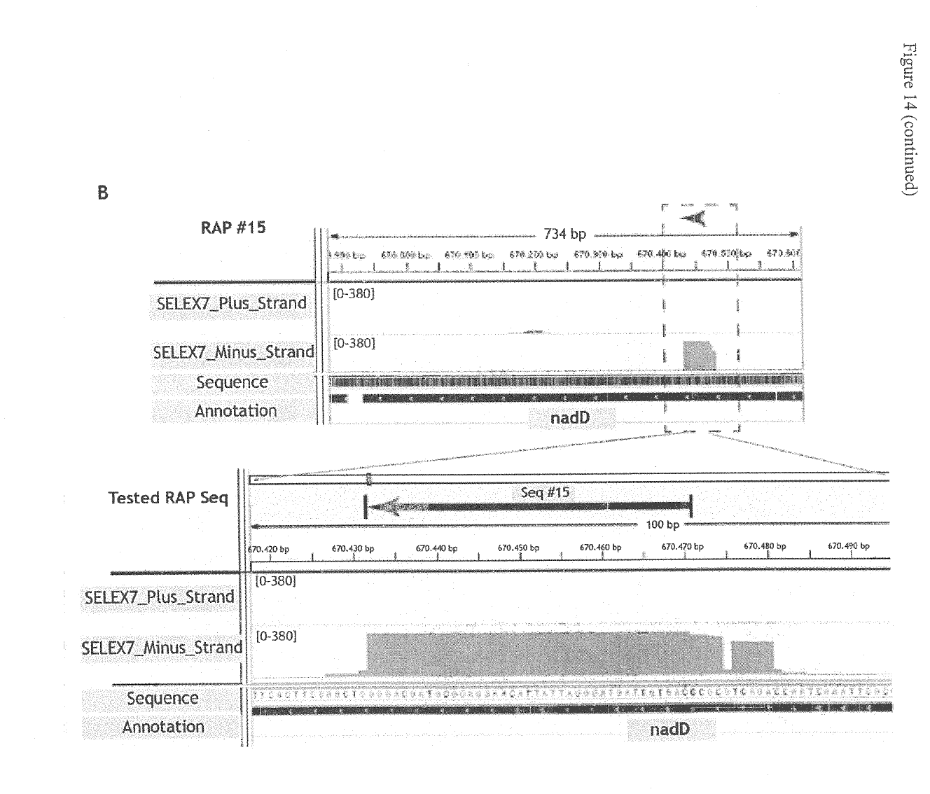

To address the physiological role of this phenomenon the activity of RAP #15 in its natural genomic context was studied. In E. coli RAP #15 is encoded within the ORF of the nicotinate-mononucleotide adenylyltransferase (nadD) gene. NadD is an essential enzyme required for de novo biosynthesis and salvage pathways of redox cofactors NAD+ and NADP+ (R. A. Mehl, C. Kinsland, T. P. Begley, J. Bacteriol. 182, 4372-4 (2000); and H. Zhang et al., Structure. 10, 69-79 (2002)). This led to the hypothesis that RAP #15 would trigger transcription termination within its nadD gene. In this case, the steady state level of the nadD mRNA upstream of RAP #15 sequence should be higher than just downstream of RAP #15. We designed RAP flanking primers to amplify the respective regions of the nadD mRNA and compared the amount of mRNA up- and downstream of RAP#15 (FIG. 13A). Total RNA was isolated from E. coli grown under different conditions and subjected to qRT-PCR. As shown in FIG. 13B, during logarithmic phase, the RNA amount just downstream of RAP#15 was reduced to 60% comparing to the amount upstream. The downstream RNA levels further decreased when cells reached the stationary phase (34% vs 100%). Challenging cells with 4 mM H₂O₂ for 20′ also resulted in major reduction of the downstream RNA (to 21%). Notably, after a brief exposure of logarithmically growing cells to BCM (75 μg/ml), the premature termination was abolished, resulting in almost identical mRNA levels upstream and downstream of RAP#15. These experiments may suggest that within its natural genomic context, RAP #15 inhibits the expression of its host nadD gene in response to stress via Rho-dependent transcription termination.

Strikingly, RAP15-mediated Rho termination occurs within the coding region of nadD. Because transcription and translation are directly coupled in bacteria (S. Proshkin, A. R. Rahmouni, A. Mironov, E. Nudler, Science. 328, 504-8 (2010)), the leading ribosome normally follows RNAP closely to prevent Rho-dependent termination. To verify that efficient translation does indeed take place during RAP15-mediated termination, constructed nad(RAP15)-GFP translational fusion was constructed, placing RAP15 in its natural genomic context in frame with the reporter gene (FIG. 23A). A similar construct, nad(rev15)-GFP, with a reverse complement sequence of RAP15 was used as a control. The results of GFP plate assays confirmed that RAP15 promotes transcription termination in spite of concomitant translation: fluorescence intensity of the RAP15 construct was significantly reduced in comparison to control (FIG. 23B). BCM restored the GFP signal, eliminating the difference between the two constructs (FIG. 23C). The fluorescence intensity was compared between the two constructs during different stages of growth in liquid culture. While no difference in growth rates was observed (FIG. 23D, upper panel), substantial reduction in GFP intensity was detected in cells carrying the RAP15 construct, starting from early exponential and becoming progressively more pronounced upon transition to the stationary phase (FIG. 23D, lower panel). Thus, RAP15 enables Rho to act within the coding region regardless of active translation. A potential mechanism by which RAP15 enables Rho-mediated termination within the coding region is pausing of the ribosome, thereby temporarily uncoupling transcription from translation. Indeed, upon binding to the surface of RNAP, RAP15 may create a physical barrier to ribosome progression (FIG. 23E).

From the data it concluded that natural RNA polymerase aptamers are specific signals impacting on transcription in a controllable manner.

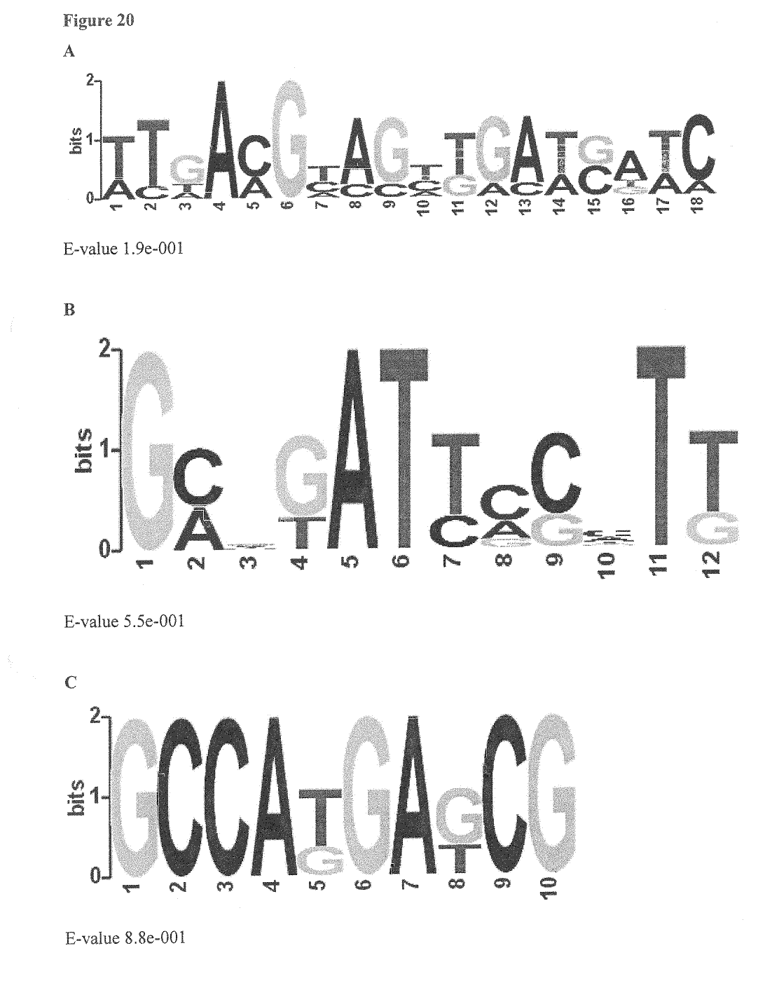

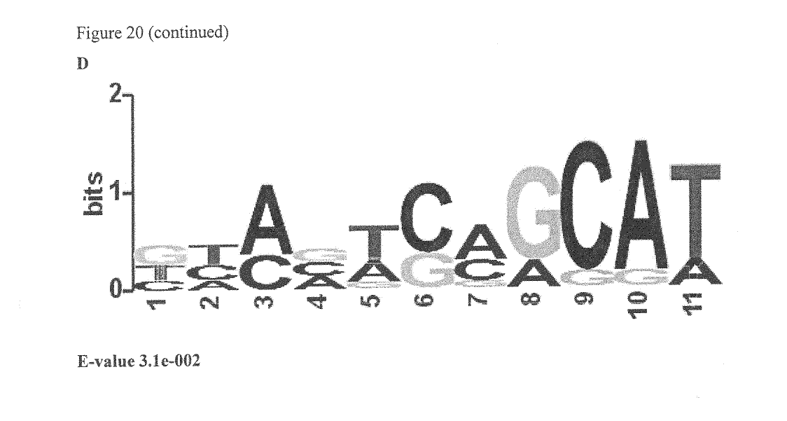

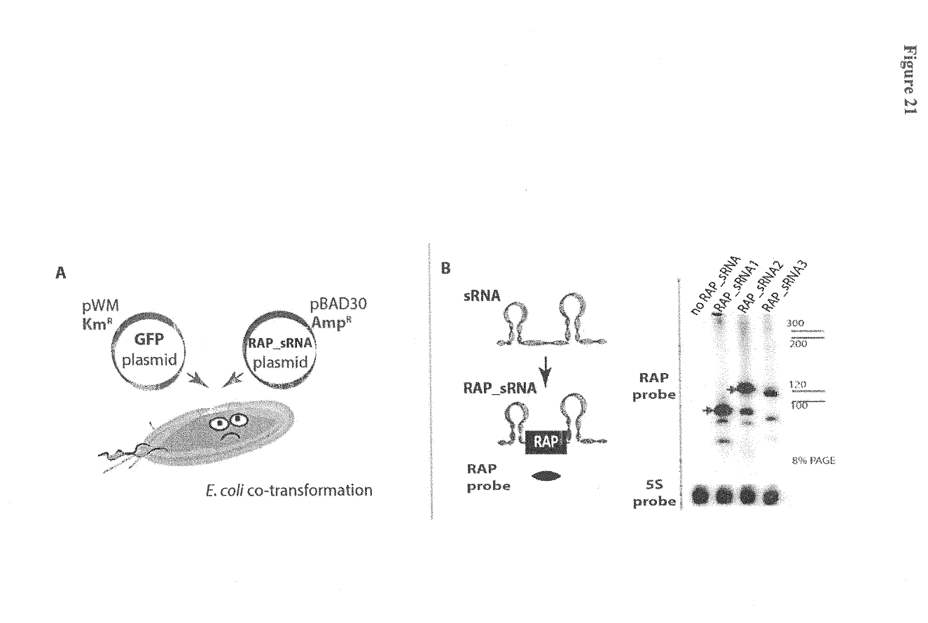

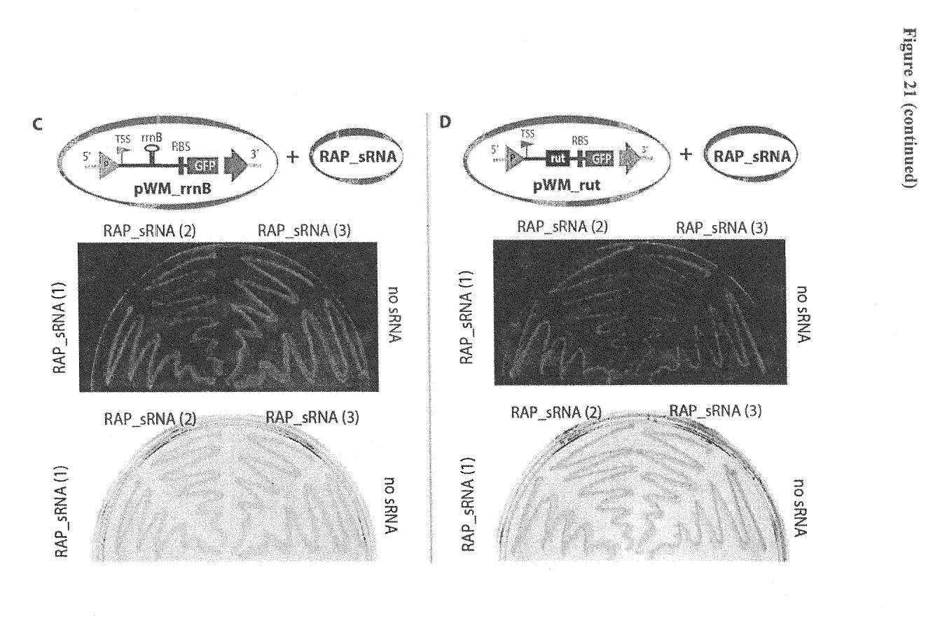

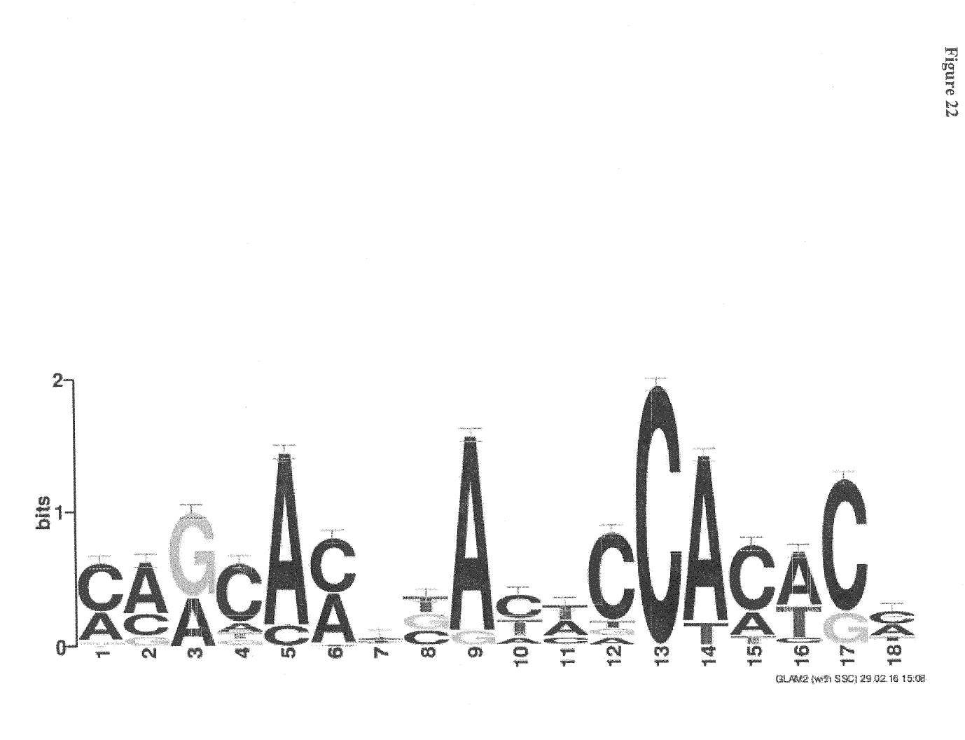

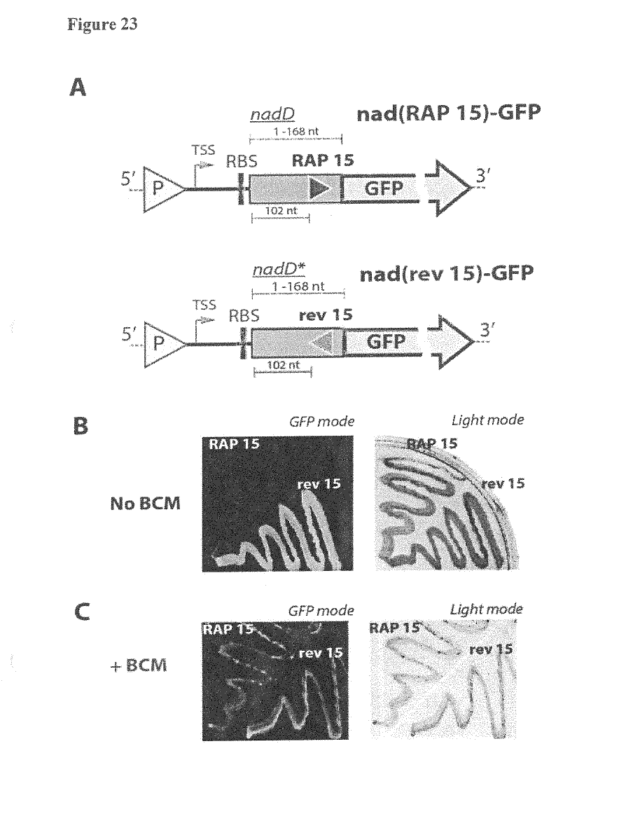

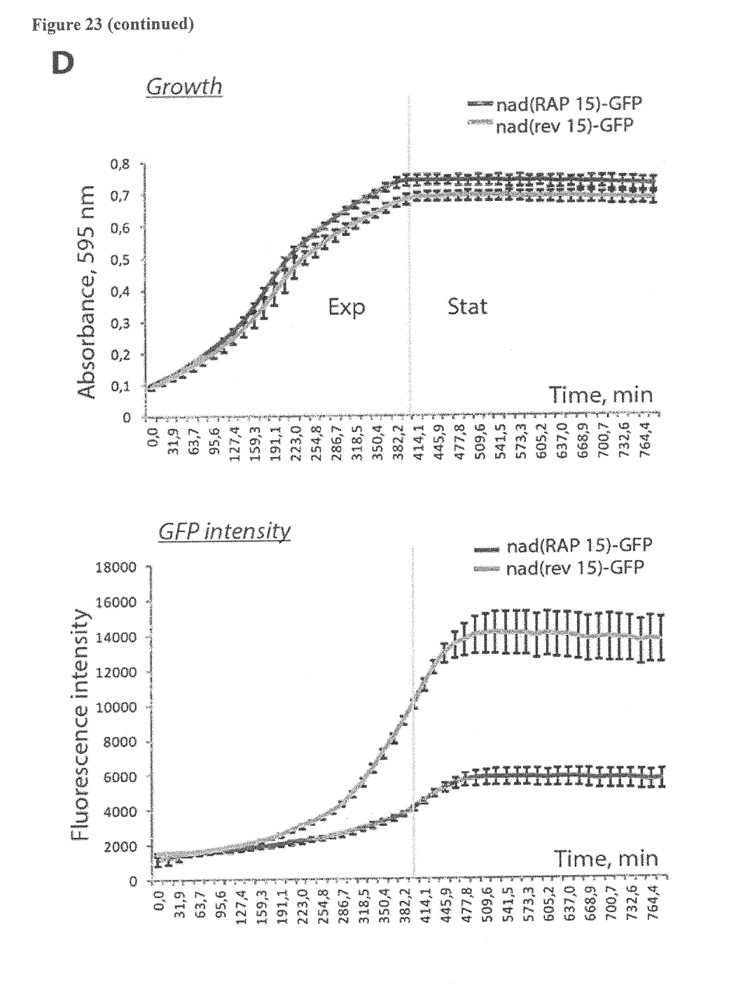

Subgroups of the identified inhibiting were analyzed for presence of consensus sequences. Using default parameters of ClustalW2 for nucleic acid sequences clusters of related sequences and the respective consensus sequences of SEQ ID NO:84 ([A/T][T/C][G/T/A]A[A/C][G[A/C/T][A/C][G/C][A/C/T][G/T][G/A][A/C][A/T][G/C][G/T/A][A/T][A/C]), SEQ ID NO:85 (G[A/C][G/T/A/C][G/T]AT[T/C][G/C/A][G/C][G/T/A/C]T[G/T], SEQ ID NO:86 (GCCA[G/T]GA[G/T]CG), and SEQ ID NO:87 ([G/T/C][A/C/T][A/C][G/C/A][G/T/A][G/C][G/C/A][G/A][G/C][G/A][A/T] could be identified. The consensus sequence of SEQ ID NO:84 is present in at least RAP ID NOs: 5039, 9533, 10822, 10070, and 1510; SEQ ID NO:85 is present in at least RAP ID NOs:1800, 1760, 4599, 3930, 683, 803, and 4599, SEQ ID NO:86 is present in at least RAP ID NOs: 1436, and 15003; and SEQ ID NO:87 is present in RAP ID NOs: 9505, 10243, 11599, 15, 1086, 2136, 6819, and 9559. Results are also shown in FIG. 20 (A: SEQ ID NO:84; B: SEQ ID NO:85; C SEQ ID NO:86; D: SEQ ID NO:87).

The presented in vitro and in vivo studies show a role of Rho in RAP-mediated transcription inhibition. Rho is a ring-shaped hexameric RNA helicase acting on the elongation complex to promote transcription termination (E. Nudler, M. E. Gottesman, Genes to Cells. 7, 755-768 (2002); and J. W. Roberts, Nature. 224, 1168-74 (1969)). In the inhibitory RAPs the C-content median value is 33,67%, whereas G-content median value is 15,69% (FIG. 19). However, the length of the tested RAP sequences does not exceed 45 nucleotides (e.g. RAP#15 is only 39 nt), which is almost twofold shorter as the required length for a rut (at least 70-80 nucleotides) (J. P. Richardson, Cell. 114, 157-9 (2003); and A. Q. Zhu, P. H. von Hippel, Biochemistry. 37, 11202-14 (1998)). Recently, tiling microarray and NGS were applied to obtain a high-resolution map of Rho-dependent termination in E. coli (J. M. Peters et al., Genes Dev. 26, 2621-33 (2012)). Only one of the twenty genes comprising the inhibitory RAPs was in the reported list of 1264 “significant transcripts” upregulated upon Rho inhibition by BCM. Therefore, it can be conclude that RAPs are not bona fide rut sites, but are likely to act via direct modulation of RNAP responsiveness to Rho. The stimulating effect of RAPs on transcriptional pausing is consistent with this hypothesis and suggests that RAPs promote Rho termination at least in part by facilitating kinetic coupling between Rho and RNAP (D. J. Jin, R. R. Burgess, J. P. Richardson, C. A. Gross, Proc. Natl. Acad Sci. U.S.A. 89, 1453-7 (1992)).

Taken together, the identification of widespread RAPs as a novel class of transcription-regulating RNA signals reveals that direct interaction of the nascent RNA with RNAP broadly impacts on the composition of the E. coli transcriptome. RAPs realize RNA's potential as a self-regulatory system.

TABLE 1
DNA sequences encoding preferred RNA-Polymerase
interacting according to the present invention

| | | | Orientation | |
|---|---|---|---|---|
| | | | of the | |
| | | | encoding | |
| SEQ | RAP | Description or | sequence | |
| ID | ID | corresponding | within | |
| NO: | (#) | E. coli gene | the gene | Sequence (5′-3′) |
| 1 | NA | Activating RAP | NA | GRHWDSATBMGRKMRVKDBAGCA |
| | | consensus | | |
| | | (conserved bases | | |
| | | underlined) | | |
| 2 | 5713 | ydhK | antisense | GACAACATGAGAACAGTTCAGCAGCACT |
| | | | | A |
| 3 | 15488 | mdtM | antisense | CAGCAATCAACCAATCCCGAAAGCATAC |
| | | | | AGACTGGTG |
| 4 | 14908 | queG | antisense | TACCGTGACGTGCCATCCAGTCCATTTC |
| | | | | GCCGTGGTATTGT |
| 5 | 9885 | xanQ | sense | GAGCACTAACAGTAAAGGAGTCAATGAT |
| | | | | GTCAGGAGAACACGTTTCATC |
| 6 | 607 | fhuC | antisense | GCTAAGACGCAGTGCAAAAGTGGTATCG |
| | | | | GAATGATTCGTGTAGCCTTGCA |
| 7 | 4962 | ydcF | antisense | GTTGCAGTGCGGATGCTGTGTGATGGCG |
| | | | | CTATACAAAAAAGTTGTCGAGTGA |
| 8 | 13404 | wzzE | sense | CGACTATGATCAGAATCGGGCCATGTTA |
| | | | | AACACCCTGAATGTTGGTC |
| 9 | 15418 | fimH | antisense | CTAAACCAGGGTAGTCCGGCAGAGTAAC |
| | | | | GGTGACATCACGATAGCACCAT |
| 10 | 602 | fhuA | antisense | ATTGAGTCGAAATCGGTATTCACCGGAT |
| | | | | TGTACAGATTGAGCCATC |
| 11 | 3439 | rlmL | antisense | AATGCGTGATTTCTTGTGCTTTCAGTCC |
| | | | | CAGTTTTG |
| 12 | 3600 | gfcD | antisense | GGAATCTGTTGGCGTGGAATAATCGGTG |
| | | | | AGACGCATCATGTTGT |
| 13 | 5151 | yddG | sense | ATCATCAGGCGATTGAAGTGGGTATGGT |
| | | | | GAACTATCTGTGG |
| 14 | 14079 | frwD | antisense | CTTGGGTTTGTTGATTACGGATATTGAG |
| | | | | CTTGTCAT |
| 15 | 14060 | frwC | antisense | CACAAATCACACAGCCCATCACCC |
| 16 | 14542 | nfrE | antisense | AGTACACCGGATCCCAGAACCACCAGCC |
| | | | | GCC |
| 17 | 11884 | feoB | antisense | CAGCCAGGTCACAATACCCAGCATCAAC |
| | | | | ATGTGGCAT |
| 18 | 3828 | mdtG | antisense | CCAAGGTACCCAAGAAGCGCCCGCAGGA |
| | | | | TCAAAAACTGCCAGATATTTT |
| 19 | 2464 | dtpD | antisense | CAGCATCACATGACCGATCGCCATCAAC |
| | | | | AACGCCC |
| 20 | 1220 | frmA | antisense | CAGCTAACCCGATGATCACCGACTGACC |
| 21 | 7866 | menE | antisense | CAGCATGCAGCAGATCCAGCGTGT |
| 22 | 10966 | yraQ | antisense | AGCAGGTACATTGTCCACAGACACTGCC |
| | | | | AGAGCCCACTGTGTCC |
| 23 | 4555 | acnA | antisense | CCAGAGTTACCAATACAGGTGGTACAAC |
| | | | | CGTATCCCACAAGGTTAA |
| 24 | 11531 | rplD | antisense | TGATGATCAGCACATCTTCCAGAGCCAT |
| | | | | GTCTTTCA |
| 25 | 15 | nadD | sense | GTCACAATCATCCCTAATAATGTTCCTC |
| | | | | CGCATCGTCCC |
| 26 | 683 | glnD | sense | TTAAACGAATGTCTGCATATATTGTGGC |
| | | | | GTATTCGCTTTGCCCTGCATCTG |
| 27 | 10070 | pgk | sense | ACCTGGTTGACGAAGCTAAACGTCTGCT |
| | | | | GACCACCTGCAACATCCCGGTTCCGTC |
| 28 | 15592 | yjjZ | antisense | TACATCAGCCCTGCAATCAGCAATCCCG |
| | | | | GCAGCAACACTCCCCAGCCA |
| 29 | 5039 | yncJ | antisense | TCATGACGCAGCTGATGATCCACATTCT |
| | | | | TTACCCACACAAATTCATGTCCTTT |
| 30 | 2136 | uspG | sense | CCACTTCACCATCGATCCTTCCCGCATT |
| | | | | AAACAACATGTCCGTTTT |
| 31 | 1800 | gcl | sense | ACATGATCACCGCGCTCTATTCCGCTTC |
| | | | | TGCTGATTCCATTCCTATTCTGTG |
| 32 | 3930 | flgL | sense | AACCCTTCTGACGATCCCATTGCTGCAT |
| | | | | CACAAGCCGTAGTTC |
| 33 | 11599 | bfr | sense | TGCCAGATCAGAACGCAGCATTTCCTCA |
| | | | | ACATCTTCACCAATGTTCA |
| 34 | 15003 | ulaG | antisense | CAAGCCACCACATCGCAAATGTGCCAGG |
| | | | | AGCGACCTGTTCTTGTTCAATTTCT |
| 35 | 9533 | gudP | antisense | CAGCGCCATAAAGCCGATGATCATCCAC |
| | | | | TCAACGTTGACGTAGTTGCAGAACACCA |
| | | | | TCACC |
| 36 | 803 | rcsF | sense | CTGTTCCATGTTAAGCAGATCCCCTGTC |
| | | | | GAACCCGTTCAAAGCACTGCACC |
| 37 | 9505 | relA | antisense | CAATGTCCATACTTAATGTCGAGAGGAT |
| | | | | CTCCACCATCTCAAC |
| 38 | 9559 | ygdH | sense | TTCGTTTTCCCAACCTGAATCTCGACAA |
| | | | | CTCCGTCCACATCAC |
| 39 | 1510 | bolA | sense | TCCAACCCGTATTCCTCGAAGTAGTGGA |
| | | | | TGAAAGCTATCGTCACAAT |
| 40 | 10243 | glcB | sense | TACCACCAAACCAACGTACAGAGCGTAC |
| | | | | AAGCCAACATTGCCC |
| 41 | 10822 | tdcE | antisense | CTTCGACATCCGCTTCGTGGTGGAAATA |
| | | | | CCCATCCAGCAGGCCGACAAGGTTGGTT |
| | | | | TTACGTACTGGATCTTCTTTGC |
| 84 | NA | inhibiting RAP | NA | WYDAMGHMSHKRMWSDWM |
| | | consensus | | |
| 85 | NA | inhibiting RAP | NA | GMNKATYVSNTK |
| | | consensus | | |
| 86 | NA | inhibiting RAP | NA | GCCAKGAKCG |
| | | consensus | | |
| 87 | NA | inhibiting RAP | NA | BHMVDSVRSRW |
| | | consensus | | |
| 88 | 4599 | yciW | antisense | CGGGCATATTGCGTGATTTGTGCCAACC |
| | | | | GATGATTACTTTCCCCTGC |
| 89 | 1436 | pgpA | antisense | CAGCGCCATGAGCGTGATCCACATACCA |
| | | | | ATAAATTCGTCCCAGACAATGCTGCCAT |
| | | | | GATCG |
| 90 | 6819 | yeeO | antisense | CCAGTCGGCAAACATTCCCATCCAGACA |
| | | | | CCAACCA |
| 91 | 1760 | ybbP | antisense | ACCATCAACCACTGACCGACGATTAGCT |
| | | | | TACGCAGTTGCGCTCGCCCTG |
| 92 | 1086 | yahJ | antisense | GCGTTTCAAATGCGCATCAACCTGCCAC |
| | | | | ACTCCCCCACA |
| 93 | yh1 | | | TCATAGACAAGACAATCCTAAGCAAAAA |
| | | | | GAACAAAGCTGTAGGCATCATACTACCT |
| | | | | GACTATC |
| 94 | yh2 | | | GAGCAAATCAGGCAGTCCTTACCCACTC |
| | | | | CCCTCCCTG |
| 95 | yh3 | | | AACTTCAAAAAAAATTACAAAAAGTGCT |
| | | | | TTCTGAACTGAACAAAAAAGAGTAAAGT |
| | | | | TAGTCGCGTAAAGTACATCT |
| 96 | yh4 | | | TTCCCAACGTGTTAGAATTATAGGCATG |
| | | | | ACCCACTGTGCC |
| 97 | yh5 | | | GTACGAGCGGACCCCACAGTTTTAAGCC |
| | | | | TAAACTTGGCCTTATGTAGAATTTCTTG |
| | | | | ATATCATTATCATCTTTGTCGTAGTCCC |
| | | | | ATC |
| 98 | yh6 | | | GCAAAATGAGTACTTAGACATGCTCTGT |
| | | | | ATTTGTGCTGTACAGTACGGTAACAC |
| 99 | yh7 | | | GAGGCACACACTCACACACCCC |
| 100 | yh8 | | | GGGAATTCGGAGCGGGATCGGTTGCATT |
| | | | | AGCTAAGCCTGAGAAGGTGATGTTGTTG |
| | | | | CC |
| 101 | yh9 | | | ATATCAGCCATGTACATCCCTGTCCCCC |
| 102 | yh10 | | | GCCCACAAGATTCCACGATAGATATCAA |
| | | | | GGATTTAAAGCTGATTCAGTGTATAGTA |
| | | | | CATC |
| 103 | yh11 | | | CCATACCAGCATGATGGATACCAAAACC |
| | | | | AAATTGAAGAATCTCCTTCC |
| 104 | yh12 | | | ATTGCTTTTCCACACGGATTCCAACCAC |
| | | | | TGC |
| 105 | yh13 | | | ATTGCTTTTCCACACGGATTCCAACCGA |
| | | | | TATCATAC |
| 106 | yh14 | | | AAGCACTACCCACCTGACAATGTCTTCA |
| | | | | TCCC |
| 107 | yh15 | | | AGCATACAAAATATACCTTTCTCAAACA |
| | | | | AGAAACATCCC |
| 108 | yh16 | | | GAGCCTTAAACACTGACTTCCCATTCCC |
| | | | | TCTCCCC |
| 109 | yh17 | | | GCAGCCCACACATCATACACACACACCC |
| | | | | C |
| 110 | yh18 | | | GCAGCTTAAACTGCCTGACCGGTAAGAA |
| | | | | ACTGACCAACATGCGTGCTTCCGGTACT |
| | | | | GACGAAGCCTTCATCC |
| 111 | yh19 | | | GCAGCCCACAAACCACACCC |
| 112 | yh20 | | | GCAGCATGAAAGTCACAGTTCTAAACAT |
| | | | | TCATTCTGTGCC |
| 113 | yh21 | | | GCAGCAGACACACGTACACACACACATA |
| | | | | CATAACCTATC |
| 114 | yh22 | | | GCAGCATATCAGCCATGTACATCCCTGT |
| | | | | CCCCC |
| 115 | yh23 | | | GCAGCAACACACACACACACACACCC |
| 116 | yh24 | | | GCAGCTTTACTTAACAGTTGAGCTAATT |
| | | | | TACACTCCCATGGATAGTGTATAAGTGT |
| | | | | TTCATTTTCGCCC |
| 117 | yh25 | | | GCAGCATGAACCAAACACCTTCCACTAG |
| | | | | TCCCATCC |
| 118 | yh26 | | | GCAGCAACACACAGCACACACATACACC |
| | | | | CACCGTACTAAGCATCC |
| 119 | yh27 | | | GAGCCAACTACATCGACCAATTTACCAG |
| | | | | AGGATTCACCATTATGATCACCATCTTC |
| | | | | C |
| 120 | yh28 | | | GCAGCAGCACTCATCGGTTCATCATCTA |
| | | | | TGTAAAGGTGCTTCTCCACCATGTGC |
| 121 | yh29 | | | GCAGCAACCACACATCTATACGGCCTTG |
| | | | | AAAACCCCATCC |
| 122 | yh30 | | | AAGCACTAAAGGCCCATTCTTGCTCTAT |
| | | | | GTATCTGTGACTTAAGATCTGCCAC |
| 123 | yh31 | | | ACGGCCACCAGACTCATTCCTGCC |
| 124 | yh32 | | | ATTGCTTTTCCACACGGATTCCAACCGC |
| | | | | TATCCAAACACTCATCCC |
| 125 | yh33 | | | GCAGCACTCACTTACACATTCACACACA |
| | | | | CAACATACACCCC |
| 126 | yh34 | | | GCAGCGTTAGTACATACCCTTCCTCACC |
| | | | | ATTGTTCCCCCATCC |
| 127 | yh35 | | | GAGCTAAGACACAGATACCCAGAAAGAT |
| | | | | CCAAACCTCTACCGCAAAGTCAAGGTAG |
| | | | | ACATTTCCC |
| 128 | yh36 | | | GAGTCCCACACACACACCACACCACC |
| 129 | yh37 | | | CACTTTGTACTTTATTGGAGTTTTCCTC |
| | | | | C |
| 130 | yh38 | | | GAGCCTACAAATTAAATCTAGCCAGAAG |
| | | | | CATTCTATCTTCC |
| 131 | yh39 | | | GCAGCAACTTCAAAAAAAATTACAAAAA |
| | | | | GTGCTTTCTGAACTGAACAAAAAAGAGT |
| | | | | AAAGTTAGTCGCGTAAAGTACATCT |
| 132 | yh40 | | | CAGATCAGGTCAGCTCAGGATAATTCAA |
| | | | | ATTCAGATAATGAGGCACAGACTAATCA |
| | | | | TAACACGAAAATTGTCATCC |
| 133 | yh41 | | | TCACATAACATTCACACATGCACACATG |
| | | | | TACCACAGCCATGCCCACATACCACAGC |
| | | | | CACGCATCATCC |
| 134 | yh42 | | | GTAGTAATCGTCTTCATTCTCATGGTGT |
| | | | | TCATCGTTCTCTATGCC |
| 135 | yh43 | | | GCAGCACTTATGGTCCATATTAGTGTCT |
| | | | | ACTACCCTGGTTATGGAAGGTTATGATA |
| | | | | CCGCACTACTGAACGCACTGTATGCC |
| 136 | yh44 | | | GAGCATATTGGACAGGTTGTACTTGATT |
| | | | | AGAAAACACAATCATGCGGTAATATTAA |
| | | | | GCCATACTCAACAGCGCCATCC |
| 137 | yh45 | | | GAGCATACGTAGTTGTAGTTTTCATTTT |
| | | | | GATGGCTTAACCTTCTTTGTCAGC |
| 138 | NA | enhancing RAP | NA | MMRCANMWBAYWCCNAM |
| | | consensus for | | |
| | | RAPs interacting | | |
| | | with eukaryotic | | |
| | | RNA-Pol II | | |
| 139 | NA | enhancing RAP | | CAN₀₋₃MMNSWNMMH |
| | | consensus | | |

Herein, nucleotide sequences are shown using the expanded letter code. In addition to the conventional GATC symbols, the expanded letter code to indicate a position within a sequence that may be flexible when defining sequences. The following table outlines the letter used and specifies which nucleotides may be included at the respective position of the sequence; e.g. if the letter H is used at position 1, this indicates that position 1 of the sequence may either be A, C, or T.

TABLE 2
Expanded Letter Code

| Letter | Nucleotide(s) included |
|---|---|
| A | A |
| T | T |
| G | G |
| C | C |
| R | G or A |
| Y | T or C |
| M | A or C |
| K | G or T |
| S | G or C |
| W | A or T |
| H | A or C or T |
| B | G or T or C |
| V | G or C or A |
| D | G or T or A |
| N | G or T or A or C |

TABLE 3
Preferred Terminator sequences:

| SEQ | | |
|---|---|---|
| ID | | |
| NO: | Name | Sequence (5′-3′) |
| 42 | rut Rho | CCCTTCCTTCTCCCCATCGCTACCTCATATCCGCAC |
| | termi- | CTCCTCAAACGCTACCTCGACCAGCCTCCCTCCCTC |
| | nation | CC |
| | se- | |
| | quence | |
| 43 | rrnB | AAACGAAAGGCTCAGTCGGAAGACTGGGCCTTTCGT |
| | intrin- | TTTATCTGT |
| | sic | |
| | termi- | |
| | nator | |
| 44 | T7t | AACCCCTTGGGGCCTCTAAACGGGTCTTGAGGGGTT |
| | intrin- | TTTTT |
| | sic | |
| | termi- | |
| | nator | |
| 45 | T7t- | tctagatcggtggAAACCCCTTGGGGCCTCTAAACGGGT |
| | tR2- | CTTGAGGGGTTTTTTTACCGTTAATAACAGGCCTGCTGG |
| | rrnB | TAATCGCAGGCCTTTTTATTTTGTTACACGCACAGCGGC |
| | triple | TAGCACGaCGACAAACGAAAGGCTCAGTCGGAAGACTGG |
| | termi- | GCCTTTCGTTTTATCTGTTAATTTTGTTTAACTTttACT |
| | nator | TTAtgtA |
| | insert | |

TABLE 4
Oligonucleotide primers, adapters and
plasmids used:

| SEQ | | |
|---|---|---|
| ID | | |
| NO: | Name | Sequence (5′-3′) |
| 46 | fixFW (primer) | TATAGGGGAATTCGGAGCGGG |
| 47 | fixRV_multi (primer) | TAGCCCGGGATCCTCGGGGCTG |
| 48 | lacZ_qPCR_FW | AGCGCGATCCCGTCGTTTTACA |
| 49 | lacZ_qPCR_RV | CAGGCTGCGCAACTGTTGGG |
| 50 | gapA_FW | GCACCACCAACTGCCTGGCT |
| 51 | gapA_RV | CGCCGCGCCAGTCTTTGTGA |
| 52 | nadA_before15_FW | ACAGGCTCTGTTTGGCGGCAC |
| 53 | nadA_before15_RV | CGCCAGCGTTTCCACGGGT |
| 54 | nadA_after15_FW | AGCGAACAGCGTGCAGCGTA |
| 55 | nadA_after15_RV | TGCGCAGTGTAAGAGGGGGC |
| 56 | P1: before RAP | TTAACCAGTAACAACAGAATTCTAGCCC |
| 57 | P2: after RAP | TTATTATCTAGAGGATCCCCGGGTGCATT |
| 58 | T7A1 promoter (small | tccagatcccgaaaatttatcaaaaagagtattgac |
| | letters) fused with | ttaaagtctaacctataggatacttacagccATCGA |
| | 20 nts for in vitro | GAGGGACACGGCGAAT |
| | transcription | |
| | (capitals) | |
| 59 | GFP_FW | TGGAGAGGGTGAAGGTGAT |
| 60 | GFP_RV | AGCATTGAACACCATAAGTCAAAG |
| 61 | 5′-phosphorylated RNA | 5′P-AAUGGACUCGUAUCACACCCGACAA-idT |
| | adapter (for 3′RACE | |
| | experiments) | |
| 62 | Forward primer 3′RACE | TTGGGCTAGAATTCTGTTGTTA |
| 63 | Reverse primer 3′RACE | TTGTCGGGTGTGATACGAGTCCATT |
| 64 | 108 nt insert to | GAATTCATCGCGTGTGCGTATTCGATCGAATTGGAT |
| | introduce additional, | CAGCTCGCGTCCAGGTAGCGAAAGCCATTTATTGAT |
| | unique XhoI and SacI | GGACCACTCGAGATCACTAGCCGAGCTCTAATTGCT |
| | restriction enzyme | AAATTC |
| | recognition sites | |
| | into plasmid pWM1015 | |
| | (resulting in pWM3110) | |
| 65 | pWM3110 | AGGCATCAAATAAAACGAAAGGCTCAGTCGAAAGAC |
| | | TGGGCCTTTCGTTTTATCTGTTGTTTGTCGGTGAAC |
| | | GCTCTCCTGAGTAGGACAAATCCGCCGGGAGCGGAT |
| | | TTGAACGTTGCGAAGCAACGGCCCGGAGGGTGGCGG |
| | | GCAGGACGCCCGCCATAAACTGCCAGGAATTAATTC |
| | | CCCAGGCATCAAATAAAACGAAAGGCTCAGTCGAAA |
| | | GACTGGGCCTTTCGTTTTATCTGTTGTTTGTCGGTG |
| | | AACGCTCTCCTGAGTAGGACAAATCCGCCGGGAGCG |
| | | GATTTGAACGTTGCGAAGCAACGGCCCGGAGGGTGG |
| | | CGGGCAGGACGCCCGCCATAAACTGCCAGGAATTAA |
| | | TTCCCCAGGCATCAAATAAAACGAAAGGCTCAGTCG |
| | | AAAGACTGGGCCTTTCGTTTTATCTGTTGTTTGTCG |
| | | GTGAACGCTCTCCTGAGTAGGACAAATCCGCCGGGA |
| | | GCGGATTTGAACGTTGCGAAGCAACGGCCCGGAGGG |
| | | TGGCGGGCAGGACGCCCGCCATAAACTGCCAGGAAT |
| | | TAATTCCCCAGGCATCAAATAAAACGAAAGGCTCAG |
| | | TCGAAAGACTGGGCCTTTCGTTTTATCTGTTGTTTG |
| | | TCGGTGAACGCTCTCCTGAGTAGGACAAATCCGCCG |
| | | GGAGCGGATTTGAACGTTGCGAAGCAACGGCCCGGA |
| | | GGGTGGCGGGCAGGACGCCCGCCATAAACTGCCAGG |
| | | AATTGGGGATCGGAAGCTTGCATGCCTGCAGGTCGA |
| | | CTCTAGAGGATCCGTTATTTTAAGTCTTAGTTTAGT |
| | | TTTTTTGGTATAATTAGAATTCatcgcgtgtgcgta |
| | | ttcgatcgaattggatcagctcgcgtccaggtagcg |
| | | aaagccatttattgatggaccactcgagatcactag |
| | | ccgagctctaattgctaaattcGGCTTATTCCCTAA |
| | | CTAACTAAAGATTAACTTTATAAGGAGGAAAAACAT |
| | | ATGAGTAAAGGAGAAGAACTTTTCACTGGAGTTGTC |
| | | CCAATTCTTGTTGAATTAGATGGTGATGTTAATGGG |
| | | CACAAATTTTCTGTCAGTGGAGAGGGTGAAGGTGAT |
| | | GCAACATACGGAAAACTTACCCTTAAATTTATTTGC |
| | | ACTACTGGAAAACTACCTGTTCCATGGCCAACACTT |
| | | GTCACTACTTTGACTTATGGTGTTCAATGCTTTTCA |
| | | AGATACCCAGATCATATGAAACGGCATGACTTTTTC |
| | | AAGAGTGCCATGCCCGAAGGTTATGTACAGGAAAGA |
| | | ACTATATTTTTCAAAGATGACGGGAACTATAAGACA |
| | | CGTGCTGAAGTCAAGTTTGAAGGTGATACACTTGTT |
| | | AATAGAATCGAGTTAAAAGGTATTGATTTTAAAGAA |
| | | GATGGAAACATTCTTGGACACAAGTTGGAATACAAC |
| | | TATAACTCACACAATGTATACATCATGGCAGACAAA |
| | | CAAAAGAATGGAATCAAAGTTAACTTCAAAATTAGA |
| | | CACAACATTGAAGATGGAAGCGTTCAACTAGCAGAC |
| | | CATTATCAACAAAATACTCCAATTGGCGATGGCCCT |
| | | GTCCTTTTACCAGACAACCATTACCTGTCCACACAA |
| | | TCTGCCCTTTCGAAAGATCCCAACGAAAAGAGAGAC |
| | | CACATGGTCCTTCTTGAGTTTGTAACAGCTGCTGGG |
| | | ATTACACATGGCATGGATGAACTATACAAATAGATC |
| | | TGCTTAATTAGCTGAGCTTGGACTCCTGTTGATAGA |
| | | TCCAGTAATGACCTCAGAACTCCATCTGGATTTGTT |
| | | CAGAACGCTCGGTTGCCGCCGGGCGTTTTTTATTGG |
| | | TGAGAATCCAAGCTAGCTTGGCGAGATTTTCAGGAG |
| | | CTAAGGAAGCTAAAATGGAGAAAAAAATAGGCCTCT |
| | | GAGCGCCGGTCGCTACCATTACCAGTTGGTCTGGTG |
| | | TCAAAAATAATAATAACCGGGCAGGCCATGTCTGCC |
| | | CGTATTTCGCGTAAGGAAATCCATTATGTACTATTT |
| | | AATTCTTGAAGACGAAAGGGCCTCGTGATACGCCTA |
| | | TTTTTATAGGTTAATGTCATGATAATAATGGTTTCT |
| | | TAGACGTCAGGTGGCACTTTTCGGGGAAATGTGCGC |
| | | GGAACCCCTATTTGTTTATTTTTCTAAATACATTCA |
| | | AATATGTATCCGCTCATGAGACAATAACCCTGATAA |
| | | ATGCTTCAATAATATTGAAAAAGGAAGAGTATGAGT |
| | | ATTCAACATTTCCGTGTCGCCCTTATTCCCTTTTTT |
| | | GCGGCATTTTGCCTTCCTGTTTTTGCTCACCCAGAA |
| | | ACGCTGGTGAAAGTAAAAGATGCTGAAGATCAGTTG |
| | | GGTGCACGAGTGGGTTACATCGAACTGGATCTCAAC |
| | | AGCGGTAAGATCCTTGAGAGTTTTCGCCCCGAAGAA |
| | | CGCTGCCTCGCGCGTTTCGGTGATGACGGTGAAAAC |
| | | CTCTGACACATGCAGCTCCCGGAGACGGTCACAGCT |
| | | TGTCTGTAAGCGGATGCCGGGAGCAGACAAGCCCGT |
| | | CAGGGCGCGTCAGCGGGTGTTGGCGGGTGTCGGGGC |
| | | GCAGCCATGACCCAGTCACGTAGCGATAGCGGAGTG |
| | | TATACTGGCTTAACTATGCGGCATCAGAGCAGATTG |
| | | TACTGAGAGTGCACCATATGCGGTGTGAAATACCGC |
| | | ACAGATGCGTAAGGAGAAAATACCGCATCAGGCGCT |
| | | CTTCCGCTTCCTCGCTCACTGACTCGCTGCGCTCGG |
| | | TCGTTCGGCTGCGGCGAGCGGTATCAGCTCACTCAA |
| | | AGGCGGTAATACGGTTATCCACAGAATCAGGGGATA |
| | | ACGCAGGAAAGAACATGTGAGCAAAAGGCCAGCAAA |
| | | AGGCCAGGAACCGTAAAAAGGCCGCGTTGCTGGCGT |
| | | TTTTCCATAGGCTCCGCCCCCCTGACGAGCATCACA |
| | | AAAATCGACGCTCAAGTCAGAGGTGGCGAAACCCGA |
| | | CAGGACTATAAAGATACCAGGCGTTTCCCCCTGGAA |
| | | GCTCCCTCGTGCGCTCTCCTGTTCCGACCCTGCCGC |
| | | TTACCGGATACCTGTCCGCCTTTCTCCCTTCGGGAA |
| | | GCGTGGCGCTTTCTCATAGCTCACGCTGTAGGTATC |
| | | TCAGTTCGGTGTAGGTCGTTCGCTCCAAGCTGGGCT |
| | | GTGTGCACGAACCCCCCGTTCAGCCCGACCGCTGCG |
| | | CCTTATCCGGTAACTATCGTCTTGAGTCCAACCCGG |
| | | TAAGACACGACTTATCGCCACTGGCAGCAGCCACTG |
| | | GTAACAGGATTAGCAGAGCGAGGTATGTAGGCGGTG |
| | | CTACAGAGTTCTTGAAGTGGTGGCCTAACTACGGCT |
| | | ACACTAGAAGGACAGTATTTGGTATCTGCGCTCTGC |
| | | TGAAGCCAGTTACCTTCGGAAAAAGAGTTGGTAGCT |
| | | CTTGATCCGGCAAACAAACCACCGCTGGTAGCGGTG |
| | | GTTTTTTTGTTTGCAAGCAGCAGATTACGCGCAGAA |
| | | AAAAAGGATCTCAAGAAGATCCTTTGATCTTTTCTA |
| | | CGGGGTCTGACGCTCAGTGGAACGAAAACTCACGTT |
| | | AAGGGATTTTGGTCATGAGATTATCAAAAAGGATCT |
| | | TCACCTAGATCCTTTTAAATTAAAAATGAAGTTTTA |
| | | AATCAATCTAAAGTATATATGAGTAAACTTGGTCTG |
| | | ACAGTTACCAATGCTTAATCAGTGAGGCACCTATCT |
| | | CAGCGATCTGTCTATTTCGTTCATCCATAGTTGCCT |
| | | GACTCCCCGTCGTGTAGATAACTACGATACGGGAGG |
| | | GCTTACCATCTGGCCCCAGTGCTGCAATGATACCGC |
| | | GAGACCCACGCTCACCGGCTCCAGATTTATCAGCAA |
| | | TAAACCAGCCAGCCGGAAGGGCCGAGCGCAGAAGTG |
| | | GTCCTGCAACTTTATCCGCCTCCATCCAGTCTATTA |
| | | ATTGTTGCCGGGAAGCTAGAGTAAGTAGTTCGCCAG |
| | | TTAATAGTTTGCGCAACGTTGTTGCCATTGCTGCAG |
| | | NGATCCACAGGACGGGTGTGGTCGCCATGATCGCGT |
| | | AGTCGATAGTGGCTCCAAGTAGCGAAGCGAGCAGGA |
| | | CTGGGCGGCGGCCAAAGCGGTCGGACAGTGCTCCGA |
| | | GAACGGGTGCGCATAGAAATTGCATCAACGCATATA |
| | | GCGCTAGCAGCACGCCATAGTGACTGGCGATGCTGT |
| | | CGGAATGGACGATATCCCGCAAGAGGCCCGGCAGTA |
| | | CCGGCATAACCAAGCCTATGCCTACAGCATCCAGGG |
| | | TGACGGTGCCGAGGATGACGATGAGCGCATTGTTAG |
| | | ATTTCATACACGGTGCCTGACTGCGTTAGCAATTTA |
| | | ACTGTGATAAACTACCGCATTAAAGCTTATGAAAAT |
| | | TTTTATAAATATTGTGAAAATAAGAATGGTTTAGCT |
| | | AAAAGTGGAAAGCCTAAAGGACTTCAAAATTTTACT |
| | | AAAAAAAGAAAATGTTATCACGAGTTTATATACGAA |
| | | ATCGGCGAAAATACTACAATGGAGCAATGCCAAGAG |
| | | CTTACGCAAAAAATTGCAGAGCTTACAGGATTTACA |
| | | CCTTTACAAGTTGTAATCCATAGAGATGAAGTAAGT |
| | | GAGAATGCTAAAGGGAAAAAACAAACCCATTATCAC |
| | | GCCCACGCAGTATTTTTTACACTTGATAATAATGGC |
| | | TTACAACTTGCTAGACGTGAAGCAAGTTTGAATAAA |
| | | GCCAATCTTAGCAAAATACAAACCCTAACCGCACAA |
| | | AGTTTAAAAATGGAGCGTGGAGCTAATCGCCACGAG |
| | | AATAACGAAAAGCAACCCCAATACATACAAGATTAT |
| | | AAAACTTACGCTCAATTTAAAGAACAAGAAAAAGCA |
| | | TTGCTTCAAAGAATACCAGGACCAGAGCATAAATTA |
| | | ACGCAAATGCCCCTAGAATTGAAAAAAAAAGAAAAA |
| | | GAGATACAAGACAAGGCTAAAGAGCTAAAATCGAAA |
| | | GAAAACGAATTACAAGCGAAAATAGAGCAACATCAA |
| | | AAACATATACAAAATTTAGAACTAGGACACGAAAGA |
| | | GCTTTAAAGGAACTTACACAAGAGTTTGAAAAGCGT |
| | | TTAAGCCTATGGAAAAACATTTTAACCTTTGGAAAA |
| | | TACAACGCCAAAGTAAGAGAAGACTATCAGTTAACA |
| | | AAGAATGCTTTTTTAATTAGCACAGATGAAAGCAGG |
| | | AGAGAAGCTAACAAAGAGCTTGAATATTTAAAATTT |
| | | GAATATCATAAAGTCAAAGATGAACGAGATAATTTA |
| | | AAAACTTTGTTTGAGGCACACAAAACAAAAAATGTT |
| | | AAATTAGAAACTCGACTAAAAGAAATAGGCAAATGG |
| | | TGTGAAAAAAATTTAAGCGTGGAGCAGTTAAAAGAA |
| | | ATATTTCCATTAAAAGCCGAAAGAATAGAAAAAGAG |
| | | CTTAAATATCAAAGAGCTTTTGAAAATTCTTTTGAA |
| | | CAAGCAAAAAGAAACGATAGAGGGTTTGGGTTTAGC |
| | | AGATAGTTTTCTTTTTTGGTGCTTTAGCAAATCGCA |
| | | CCTGTTAAAGTAATGGATTTGCGAAAAGGGGGTGCG |
| | | GGGGCTGTCTGAGTCGTAGCACGGAAGACGGACAAA |
| | | GCCCACCGCCCTAGAAAAAAAATTACTTCAACTTTT |
| | | TTTGGTGTTTGTCTCTCGCGCGCGTACCGTACGCGA |
| | | ATTTTTTTATTTTTTATATATTTTTATATATTTTTA |
| | | GGGGGCTTTCTAAGCCCTATATGATGGGGGCTAGCG |
| | | AAATATTAAGGGTAGAAATGGTAGGTATTAAGGGTA |
| | | GAAAATGGTAGGTATTAAGGGTAGAAATGGTAGGTA |
| | | TTAAGGGTAGAAATGGTAGGTATTAAGGGTAGAAAT |
| | | GGTAGGTATTAAGGGTAGAAACTATTGACAATCTAT |
| | | TTTTAATATGCTACAATAAAAAATCCGCCTTGAGTT |
| | | TTCACTCTTGGCGGAAATTTATCAATCAAGGCTAAC |
| | | TATGAAACGACATAGTAAGCGTTTTGTGAGATTATA |
| | | CCTAAATTTTAGGTTTTTTAAGCTTAAAGTTGAAGT |
| | | ATTTTTTCTAACAAAACCGCAGGGGCGGAAAAGCCC |
| | | CTTTGCCTTGATTGTCTTACTTTGGATATGCAAATG |
| | | AGCGAAATAGTTAAATATCACAACGATTTTAATAAA |
| | | ATTCAGTTGCCTAGCTTTACAGAGCAAGAGCAGAAC |
| | | CTTTTTTTGTTTTATATCTTTGCAAGAATTAAAGAA |
| | | AAAGGCATAGACAATGTAATTAATCTCTATGCTAAT |
| | | GATTTTAAAATTAATGAAAAACTTTCTAACTCAAGA |
| | | GAATATTTAACAGAAAATATCAATAGCTTAAAATAT |
| | | AAATTTTTCAAGGCAAATTTTAAACAAATCATAGAA |
| | | ACAAAGACAGAAGTTATACACAAATATGTAAATTTG |
| | | TTTGAAACAATGGAAATACACTATGTTAAAAGAAAT |
| | | CCTGATGATGGATATGATGAAAGCACTTTATTTAAC |
| | | AGAATAACCCTAAAAATAAACCCTGATTTTGCTTAT |
| | | TTAGTCAATCAACTAACTACTAATTACAGCTTTGAG |
| | | CTTGAAGAATTTATAGCCCTTAGCGGTAAATATGCT |
| | | AAAACACTTTACCGCCTTTTAAAGCAATTTAGAACC |
| | | ACTGGCAAAGCTTATTTTGAGTGGGACGAGTTTTGT |
| | | AAAGTTATGGATATACCACAAGATTATAGACAAAGC |
| | | GAAGTCGATAAATGGATTTTAAACCTGCTATCAAAG |
| | | AACTTTCTAAAGAACAGCAATCTTTTTGACCAAATT |
| | | AGAGTGCCTTTTAAAAATCTTGCTTATGAAAAAGAA |
| | | AAAACCGCAGGCGTGGGCGTGGCGGCAAAGTTTCAG |
| | | GCATTAGCTTTACTTTTAAACCTGAAAATATCGAAA |
| | | TGCAAAAGCTAGAAAATGAAAGTCAAAAAATAATGA |
| | | GCGATGAGCAAAAATATTTAAAGATTTTAAACAATA |
| | | TGAAACTTAATCAAGCTAGATTTAATTATAATGACA |
| | | AGCTTTGGCAGTTTAACGATTTTGATTTTAATGAAT |
| | | TTAAAATTATTGCAGTAGAGCTTGTAAGAGATGAAT |
| | | ACGAGAATTTAAACTTTGGAAATCATATGCACTTTA |
| | | ATGCTAAAAATCAAGAGCAGTTTTTTAAATGATTGA |
| | | TACTTTAGGAATGGGATTAGGTAAAATTTGCTATAA |
| | | TTATATCTTTGAAGGATACAAGCTATCCGCCACTTG |
| | | TGCCAAGTGTCGAGCTTGTAAGGGGTGCAACCCCTT |
| | | AACCCCACTAATAAAAATCAACTAAATCAGTGAATG |
| | | ATTTTTGAATTTTATTAAAACACCAAAAAAGGAAAA |
| | | TTTAAAAATGCAAAAACATATCCCGAAGTGCATAGC |
| | | TTAGAAGAAAGCCTTGCGATACTTAAAAAATATAAA |
| | | GATGATTTGACTAAAGAGCAATACGAAGCCATAAGA |
| | | TCAAATATAGGTAATTTTGCAATAGAAGATATGTTT |
| | | TTGAATGAAAAAGATATTATTGATAATGTTAAAATT |
| | | ATAAAAGGCGAAGCAACAGCTAATGAATGTATTGTT |
| | | GCATTAAAGAAAGAATGGGGAGTATGAATGCAAAAT |
| | | GTCAAAAGTTGCCACCGCTCATTGCAATCCTAAAAA |
| | | ACAACCTGCATTAAACCACAATGATAGAACCAACGA |
| | | TAATGCTAAGACAATCACTAAAGAACTTACACATTT |
| | | AAATGAATACTCTTGCACTAGCGATGAAGTGCGTAA |
| | | GAACATAGAAAGGCTTTATAAAAAAGCTTTTTAGAC |
| | | ATCTAAATCTAGGTACTAAAACAATTCATCCAGTAA |
| | | AATATAATATTTTATTTTCTCCCAATCAGGCTTGAT |
| | | CCCCAGTAAGTCAAAAAATAGCTCGACATACTGTTC |
| | | TTCCCCGATATCCTCCCTGATCGACCGGACGCAGAA |
| | | GGCAATGTCATACCACTTGTCCGCCCTGCCGCTTCT |
| | | CCCAAGATCAATAAAGCCACTTACTTTGCCATCTTT |
| | | CACAAAGATGTTGCTGTCTCCCAGGTCGCCGTGGGA |
| | | AAAGACAAGTTCCTCTTCGGGCTTTTCCGTCTTTAA |
| | | AAAATCATACAGCTCGCGCGGATCTTTAAATGGAGT |
| | | GTCTTCTTCCCAGTTTTCGCAATCCACATCGGCCAG |
| | | ATCGTTATTCAGTAAGTAATCCAATTCGGCTAAGCG |
| | | GCTGTCTAAGCTATTCGTATAGGGACAATCCGATAT |
| | | GTCGATGGAGTGAAAGAGCCTGATGCACTCCGCATA |
| | | CAGCTCGATAATCTTTTCAGGGCTTTGTTCATCTTC |
| | | ATACTCTTCCGAGCAAAGGACGCCATCGGCCTCACT |
| | | CATGAGCAGATTGCTCCAGCCATCATGCCGTTCAAA |
| | | GTGCAGGACCTTTGGAACAGGCAGCTTTCCTTCCAG |
| | | CCATAGCATCATGTCCTTTTCCCGTTCCACATCATA |
| | | GGTGGTCCCTTTATACCGGCTGTCCGTCATTTTTAA |
| | | ATATAGGTTTTCATTTTCTCCCACCAGCTTATATAC |
| | | CTTAGCAGGAGACATTCCTTCCGTATCTTTTACGCA |
| | | GCGGTATTTTTCGATCAGTTTTTTCAATTCCGGTGA |
| | | TATTCTCATTTTAGCCATTTATTATTTCCTTCCTCT |
| | | TTTCTACAGTATTTAAAGATACCCCAAGAAGCTAAT |
| | | TATAACAAGACGAACTCCAATTCACTGTTCCTTGCA |
| | | TTCTAAAACCTTAAATACCAGAAAACAGCTTTTTCA |
| | | AAGTTGTTTTCAAAGTTGGCGTATAACATAGTATCG |
| | | ACGGAGCCGATTTTGAAACCACAATTATGATAGAAT |
| | | TTACAAGCTATAAGGTTATTGTCCTGGGTTTCAAGC |
| | | ATTAGTCCATGCAAGTTTTTATGCTTTGCCCATTCT |
| | | ATAGATATATTGATAAGCGCGCTGCCTATGCCTTGC |
| | | CCCCTGAAATCCTTACATACGGCGATATCTTCTATA |
| | | TAAGCGTACCGGTTCCAATTTTTTCGCAGTTTAACT |
| | | TTTCCGACGCATTTATCGTCTTGGTAGTAAAGATAT |
| | | ATTATATATTATCTTATCAGTATTGTCAATATATTC |
| | | AAGGCAATCTGCCTCCTCATCCTCTTCATCCTCTTC |
| | | GTCTTGGTAGCTTTTTAAATATGGCGCTTCATAGAG |
| | | TAATTCTGTAAAGGTCCAATTCTCGTTTTCATACCT |
| | | CGGTATAATCTTACCTATCACCTCAAATGGTTCGCT |
| | | GGGTTTATCGATGATAAGCTGTCAAACATGA |
| 66 | promoter of pWM3110 | TTTAAGTCTTAGTTTAGTTTTTTTGGTATAAT |
| | (constitutive promoter) | |
| 67 | arabinose-inducible | GTTTCTCCATACCCGTTTTTTTGGGCTAGC |
| | promoter | |
| 68 | Insert for integration | GAGCTCTGCCGTACCGTCTATCAAACTCAACGACCC |
| | of rut into pWM3100 | CTTCCTTCTCCCCATCGCTACCTCATATCCGCACCT |
| | (rut sequence in bold) | CCTCAAACGCTACCTCGACCAGCCTCCCTCCCTCCC |
| | | GTGCTC |
| 69 | Insert for integration | GAGCTCTGCCGTACCGTCTATCAAACTCAACGACAC |
| | of rrnB intrinsic | CATAGCGTCCTGGATCATTAACGTGATACGCAGATA |
| | terminator into pWM3100 | CCGTTGCAccACTCTGITCACAGCGGCTAGCACGTC |
| | (rrnB sequence in bold) | GACAAACGAAAGGCTCAGTCGGAAGACTGGGCCTTT |
| | | CGTTTTATCTGTTAATTTTGTTTAACTTTAGGAGTG |
| | | CTC |
| 70 | Insert for integration | GAGCTCTGCCGTACCGTCTATCAAACTCAACGACAC |
| | of T7t intrinsic | CATAGCGTCCTGGATCATTAACGTGATACGCAGATA |
| | terminator into pWM3100 | CCGTTGCAccACTCTGITCACAGCGGCTAGCACGTC |
| | (T7t sequence in bold) | GACAACCCCTTGGGGCCTCTAAACGGGTCTTGAGGG |
| | | GTTTTTTTTAATTTTGTTTAACTTTAGGAGTGCTC |
| 71 | Insert for integration | GAGCTCtctagatcggtggAAACCCCTTGGGGCCTC |
| | of the three intrinsic | TAAACGGGTCTTGAGGGGTTTTTTTACCGTTAATAA |
| | terminators T7t, tR2 | CAGGCCTGCTGGTAATCGCAGGCCTTTTTATTTTGT |
| | and rrnB into pWM 3100 | TACACGCACAGCGGCTAGCACGaCGACAAACGAAAG |
| | (T7t - tR2 - rrnB | GCTCAGTCGGAAGACTGGGCCTTTCGTTTTATCTGT |
| | sequences in bold) | TAATTTTGTTTAACTTttACTTTAtgtAGTGCTC |
| 78 | +70 RBS | TAATAACTATACGTATGGTAATCGCAGGCCTTATTC |
| | | CGAGCTCAATAATTGTTAACTTTAAGAAGGAGGA |
| 79 | lacZ gene in pBAD13 | ATGTCGTTTACTTTGACCAACAAGAACGTGATTTTC |
| | | GTTGCCGGTCTGGGAGGCATTGGTCTGGACACCAGC |
| | | AAGGAGCTGCTCAAGCGCGATCCCGTCGTTTTACAA |
| | | CGTCGTGACTGGGAAAACCCTGGCGTTACCCAACTT |
| | | AATCGCCTTGCAGCACATCCCCCTTTCGCCAGCTGG |
| | | CGTAATAGCGAAGAGGCCCGCACCGATCGCCCTTCC |
| | | CAACAGTTGCGCAGCCTGAATGGCGAATGGCGCTTT |
| | | GCCTGGTTTCCGGCACCAGAAGCGGTGCCGGAAAGC |
| | | TGGCTGGAGTGCGATCTTCCTGAGGCCGATACTGTC |
| | | GTCGTCCCCTCAAACTGGCAGATGCACGGTTACGAT |
| | | GCGCCCATCTACACCAACGTGACCTATCCCATTACG |
| | | GTCAATCCGCCGTTTGTTCCCACGGAGAATCCGACG |
| | | GGTTGTTACTCGCTCACATTTAATGTTGATGAAAGC |
| | | TGGCTACAGGAAGGCCAGACGCGAATTATTTTTGAT |
| | | GGCGTTAACTCGGCGTTTCATCTGTGGTGCAACGGG |
| | | CGCTGGGTCGGTTACGGCCAGGACAGTCGTTTGCCG |
| | | TCTGAATTTGACCTGAGCGCATTTTTACGCGCCGGA |
| | | GAAAACCGCCTCGCGGTGATGGTGCTGCGTTGGAGT |
| | | GACGGCAGTTATCTGGAAGATCAGGATATGTGGCGG |
| | | ATGAGCGGCATTTTCCGTGACGTCTCGTTGCTGCAT |
| | | AAACCGACTACACAAATCAGCGATTTCCATGTTGCC |
| | | ACTCGCTTTAATGATGATTTCAGCCGCGCTGTACTG |
| | | GAGGCTGAAGTTCAGATGTGCGGCGAGTTGCGTGAC |
| | | TACCTACGGGTAACAGTTTCTTTATGGCAGGGTGAA |
| | | ACGCAGGTCGCCAGCGGCACCGCGCCTTTCGGCGGT |
| | | GAAATTATCGATGAGCGTGGTGGTTATGCCGATCGC |
| | | GTCACACTACGTCTGAACGTCGAAAACCCGAAACTG |
| | | TGGAGCGCCGAAATCCCGAATCTCTATCGTGCGGTG |
| | | GTTGAACTGCACACCGCCGACGGCACGCTGATTGAA |
| | | GCAGAAGCCTGCGATGTCGGTTTCCGCGAGGTGCGG |
| | | ATTGAAAATGGTCTGCTGCTGCTGAACGGCAAGCCG |
| | | TTGCTGATTCGAGGCGTTAACCGTCACGAGCATCAT |
| | | CCTCTGCATGGTCAGGTCATGGATGAGCAGACGATG |
| | | GTGCAGGATATCCTGCTGATGAAGCAGAACAACTTT |
| | | AACGCCGTGCGCTGTTCGCATTATCCGAACCATCCG |
| | | CTGTGGTACACGCTGTGCGACCGCTACGGCCTGTAT |
| | | GTGGTGGATGAAGCCAATATTGAAACCCACGGCATG |
| | | GTGCCAATGAATCGTCTGACCGATGATCCGCGCTGG |
| | | CTACCGGCGATGAGCGAACGCGTAACGCGAATGGTG |
| | | CAGCGCGATCGTAATCACCCGAGTGTGATCATCTGG |
| | | TCGCTGGGGAATGAATCAGGCCACGGCGCTAATCAC |
| | | GACGCGCTGTATCGCTGGATCAAATCTGTCGATCCT |
| | | TCCCGCCCGGTGCAGTATGAAGGCGGCGGAGCCGAC |
| | | ACCACGGCCACCGATATTATTTGCCCGATGTACGCG |
| | | CGCGTGGATGAAGACCAGCCCTTCCCGGCTGTGCCG |
| | | AAATGGTCCATCAAAAAATGGCTTTCGCTACCTGGA |
| | | GAGACGCGCCCGCTGATCCTTTGCGAATACGCCCAC |
| | | GCGATGGGTAACAGTCTTGGCGGTTTCGCTAAATAC |
| | | TGGCAGGCGTTTCGTCAGTATCCCCGTTTACAGGGC |
| | | GGCTTCGTCTGGGACTGGGTGGATCAGTCGCTGATT |
| | | AAATATGATGAAAACGGCAACCCGTGGTCGGCTTAC |
| | | GGCGGTGATTTTGGCGATACGCCGAACGATCGCCAG |
| | | TTCTGTATGAACGGTCTGGTCTTTGCCGACCGCACG |
| | | CCGCATCCAGCGCTGACGGAAGCAAAACACCAGCAG |
| | | CAGTTTTTCCAGTTCCGTTTATCCGGGCAAACCATC |
| | | GAAGTGACCAGCGAATACCTGTTCCGTCATAGCGAT |
| | | AACGAGCTCCTGCACTGGATGGTGGCGCTGGATGGT |
| | | AAGCCGCTGGCAAGCGGTGAAGTGCCTCTGGATGTC |
| | | GCTCCACAAGGTAAACAGTTGATTGAACTGCCTGAA |
| | | CTACCGCAGCCGGAGAGCGCCGGGCAACTCTGGCTC |
| | | ACAGTACGCGTAGTGCAACCGAACGCGACCGCATGG |
| | | TCAGAAGCCGGGCACATCAGCGCCTGGCAGCAGTGG |
| | | CGTCTGGCGGAAAACCTCAGTGTGACGCTCCCCGCC |
| | | GCGTCCCACGCCATCCCGCATCTGACCACCAGCGAA |
| | | ATGGATTTTTGCATCGAGCTGGGTAATAAGCGTTGG |
| | | CAATTTAACCGCCAGTCAGGCTTTCTTTCACAGATG |
| | | TGGATTGGCGATAAAAAACAACTGCTGACGCCGCTG |
| | | CGCGATCAGTTCACCCGTGCACCGCTGGATAACGAC |
| | | ATTGGCGTAAGTGAAGCGACCCGCATTGACCCTAAC |
| | | GCCTGGGTCGAACGCTGGAAGGCGGCGGGCCATTAC |
| | | CAGGCCGAAGCAGCGTTGTTGCAGTGCACGGCAGAT |
| | | ACACTTGCTGATGCGGTGCTGATTACGACCGCTCAC |
| | | GCGTGGCAGCATCAGGGGAAAACCTTATTTATCAGC |
| | | CGGAAAACCTACCGGATTGATGGTAGTGGTCAAATG |
| | | GCGATTACCGTTGATGTTGAAGTGGCGAGCGATACA |
| | | CCGCATCCGGCGCGGATTGGCCTGAACTGCCAGCTG |
| | | GCGCAGGTAGCAGAGCGGGTAAACTGGCTCGGATTA |
| | | GGGCCGCAAGAAAACTATCCCGACCGCCTTACTGCC |
| | | GCCTGTTTTGACCGCTGGGATCTGCCATTGTCAGAC |
| | | ATGTATACCCCGTACGTCTTCCCGAGCGAAAACGGT |
| | | CTGCGCTGCGGGACGCGCGAATTGAATTATGGCCCA |
| | | CACCAGTGGCGCGGCGACTTCCAGTTCAACATCAGC |
| | | CGCTACAGTCAACAGCAACTGATGGAAACCAGCCAT |
| | | CGCCATCTGCTGCACGCGGAAGAAGGCACATGGCTG |
| | | AATATCGACGGTTTCCATATGGGGATTGGTGGCGAC |
| | | GACTCCTGGAGCCCGTCAGTATCGGCGGAATTACAG |
| | | CTGAGCGCCGGTCGCTACCATTACCAGTTGGTCTGG |
| | | TGTCAAAAATAATAATAA |
| 80 | cobC-derived +99 nt | TGCATTCTCGACCCCGATGGCACGGCTATTGAGGAC |
| | insert | GCGTAGCGTCGCGAATTTTTGGTTGATATCAATGGC |
| | | GCTCCAACACCCCTGGTCAACGCGAAA |
| 81 | +49 nt insert | TTTGTTACTGGTTAACCTGAAACGCCAGTCTGCCCA |
| | | TACGCCACTGCGT |
| 82 | Forward primer: | ATGCTAGCACCCGTTTTTTTGGGCTAGAAT |
| | pET21_Insert_FW_NheI | |
| 83 | Reverse primer: | ATAAGCTTTTATTATTATTTTTGACACCAGAC |
| | pET21_Insert_RV_HindIII | |

TABLE 5
Active variants of SEQ ID NO: 2

| SEQ | | | | |
|---|---|---|---|---|
| ID | | Sequence | | Length, |
| NO: | RAP/Mutant ID | name | Sequence (5′-3′) | nt |
| 1 | NA | consensus | GRHWDSATBMGRKMRVKDBAGCA | |
| 2 | 5713 | ydhK | GACAACATGAGAACAGTTCAGCAGCACTA | 29 |
| 72 | 5713_mut_loop2 | ydhK_loop2 | GACAACATGAGGGCGGTTCAGCAGCACTA | 29 |
| 73 | 5713_mut_2gg | ydhK_2gg | GACGGCATGAGGGCAGTTCAGCAGCACTA | 29 |
| 74 | 5713_mut_3gg | ydhK_3gg | GACGGCATGAGGGCAGGGCAGCAGCACTA | 29 |
| 75 | 5713_mut_0-6 | ydhK_0-6 | GACAACATGAGAACAGTTCAGCA | 23 |

Mutations with respect to SEQ ID NO:2 are underlined. In SEQ ID NO:1 and 2 nucleotides without alternatives in the consensus sequence of SEQ ID NO:1 are in italics. SEQ ID NO:75 is shortened by 6 nt at the 3′-end as compared to SEQ ID NO:2. It hence, only comprises the sequence corresponding to the consensus sequence of SEQ ID NO:1.

TABLE 6
Inactive variants of SEQ ID NO: 2:

| SEQ | | | | |
|---|---|---|---|---|
| ID | RAP/Mutant | Sequence | | Length, |
| NO: | ID | name | Sequence (5′-3′) | nt |
| 76 | 5713_mut_ste | ydhK_stem3 | GACAACAACAGAACAGTTGTGCAGCACTA | 29 |
| | m3 | | | |
| 77 | 5713_mut_no | ydhK_noStem | GACAACACCCGAACAGTCCCGCAGCACTA | 29 |
| | Struct | | | |

TABLE 7

| | | p- | | | |
|---|---|---|---|---|---|
| Name | Start* | value | Sites | | |
| ydhK | 1 | 2.40e− | | GACAACATGAGGGCGGTTCAGCA | GCACTA |
| _loop | | 12 | | | |
| 2 | | | | | |
| ydhK | 1 | 7.18e− | | GACGGCATGAGGGCAGTTCAGCA | GCACTA |
| _2gg | | 12 | | | |
| ydhK | 1 | 2.43e− | | GACAACATGAGAACAGTTCAGCA | |
| _0-6 | | 11 | | | |
| ydhK | 1 | 2.43e− | | GACAACATGAGAACAGTTCAGCA | GCACTA |
| | | 11 | | | |
| ydhK | 1 | 9.71e− | | GACGGCATGAGGGCAGGGCAGCA | GCACTA |
| _3gg | | 10 | | | |
| xanQ | 18 | 9.80e− | AACAGTAAAG | GAGTCAATGATGTCAGGAGAACA | CGTTTCATC |
| | | 06 | | | |
| fimH | 24 | 4.17e− | GTCCGGCAGA | GTAACGGTGACATCACGATAGCA | CCAT |
| | | 05 | | | |
| fhuC | 30 | 2.88e− | AAGTGGTATC | GGAATGATTCGTGTAGCCTTGCA | TC |
| | | 04 | | | |
| queG | 9 | 3.57e− | TACCGTGA | CGTGCCATCCAGTCCATTTCGCC | GTGGTATTGT |
| | | 04 | | | |
| mdtM | 3 | 5.02e− | CA | GCAATCAACCAATCCCGAAAGCA | TACAGACTGG |
| | | 04 | | | |
| wzzE | 8 | 6.11e− | CGACTAT | GATCAGAATCGGGCCATGTTAAA | CACCCTGAAT |
| | | 04 | | | |
| frwD | 8 | 6.94e- | CTTGGGT | TTGTTGATTACGGATATTGAGCT | TGTCAT |
| | | 04 | | | |
| fhuA | 21 | 7.87e− | AATCGGTATT | CACCGGATTGTACAGATTGAGCC | ATC |
| | | 04 | | | |
| gfcD | 16 | 1.06e− | CTGTTGGCGT | GGAATAATCGGTGAGACGCATCA | TGTTGT |
| | | 03 | | | |
| yddG | 11 | 1.20e− | ATCATCAGGC | GATTGAAGTGGGTATGGTGAACT | ATCTGTGG |
| | | 03 | | | |
| ydcF | 18 | 1.97e− | GCAGTGCGGA | TGCTGTGTGATGGCGCTATACAA | AAAAGTTGTC |
| | | 03 | | | |
| rlmL | 8 | 6.23e− | AATGCGT | GATTTCTTGTGCTTTCAGTCCCA | GTTTTG |
| | | 03 | | | |

The present invention while not narrowing the above, in particular relates to the following items:
Item 1: A RNA-molecule comprising a RNA-polymerase binding aptamer, wherein said RNA-polymerase binding aptamer has a length of 15 to 60 nt, preferably 20 to 50 nt, more preferably 23 to 50 nt, and wherein said RNA-Polymerase binding aptamer binds to a RNA-polymerase with a K_{D} of 50 nM or lower, preferably with a K of 10 nM or lower.
Item 2: The RNA-molecule according to item 1, wherein said RNA-polymerase is a prokaryotic RNA-polymerase, preferably a bacterial RNA-polymerase, more preferably a bacterial RNA-polymerase derived from gram negative bacteria, even more preferably the RNA-polymerase of Escherichia coli.
Item 3: The RNA-molecule according to item 1 or item 2, wherein the RNA-polymerase binding aptamer interacts with the holoenzyme of the RNA-polymerase, preferably the RNA-polymerase core and the sigma 70.
Item 4: The RNA-molecule according to any one of items 1 to 3, wherein the aptamer comprises a sequence according to SEQ ID NO:1.
Item 5: The RNA-molecule according to item 4, wherein the RNA-polymerase binding aptamer is a transcription enhancing RNA-polymerase binding aptamer.
Item 6: The RNA-molecule according to item 5, wherein the transcription enhancing RNA-polymerase binding aptamer increases expression of a sequence to be expressed, wherein the increase of expression is at least 10% as compared to the expression of said sequence to be expressed when not comprising said RNA-polymerase binding aptamer, preferably by at least 20%, more preferably at least 100%.
Item 7: The RNA-molecule according to any one of items 3 to 6, wherein the transcription enhancing RNA-polymerase binding aptamer is encoded by a sequence of SEQ ID NO:1; preferably encoded by a sequence having at least 80% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, and SEQ ID NO:75.
Item 8: The RNA-molecule according to any one of items 1 to 3, wherein the RNA-polymerase binding aptamer is an inhibiting RNA-polymerase binding aptamer.
Item 9: The RNA-molecule according to item 8, wherein the inhibiting RNA-polymerase binding aptamer reduces expression of a sequence to be expressed, wherein the reduction of expression is at least 30% as compared to the expression of said sequence to be expressed when not comprising said RNA-polymerase binding aptamer, preferably by at least 20%, even more preferably by at least 50%.
Item 10: The RNA-molecule according to item 8 or item 9 wherein the inhibiting RNA-polymerase binding aptamer has a C-content of more than 27%, preferably more than 30%, more preferably more than 31%, and wherein the RNA-polymerase binding aptamer has a G-content of less than 23%, preferably less than 20%, more preferably less than 17%.
Item 11: The RNA-molecule according to any one of items 8 to 10, wherein the inhibiting RNA-polymerase binding aptamer is encoded by a sequence selected from the group consisting of SEQ ID NO:84, SEQ ID NO:85, SEQ ID NO:86, and SEQ ID NO:87, preferably the inhibiting RNA-polymerase binding aptamer is encoded by a sequence having at least 80% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92.
Item 12: A DNA-molecule comprising a sequence encoding an RNA-polymerase binding aptamer as defined in any one of items 1 to 11.
Item 13: The DNA-molecule according to item 12, wherein the DNA-molecule comprises an expression cassette, said expression cassette comprising a promoter, said sequence encoding the RNA-polymerase binding aptamer, and a sequence to be expressed or a multiple cloning site for introducing said sequence to be expressed, wherein said promoter, the sequence encoding said RNA-polymerase binding aptamer and said sequence to be expressed are operatively linked, preferably the sequence encoding said RNA-polymerase binding aptamer, and said sequence to be expressed or said multiple cloning site are downstream of said promoter.
Item 14: The DNA-molecule according to item 13, wherein the sequence to be expressed codes for a protein and the RNA-polymerase binding aptamer is located upstream of the open reading frame.
Item 15: The DNA-molecule according to item 14, wherein the said expression cassette comprises two or more open reading frames coding for one or more proteins to be expressed, wherein the expression cassette comprises for each open reading frame a sequence encoding a RNA-polymerase binding aptamer as defined in any one of items 1 to 11 operatively linked to said open reading frame, preferably the two or more open reading frames are separated by a sequence encoding said one or more RNA-polymerase binding aptamer and a translation initiation site, both operatively linked to said open reading frame.
Item 16: The DNA-molecule according to any one of items 12 to 15, wherein said RNA-polymerase binding aptamer is a transcription enhancing RNA-polymerase binding aptamer according to any one of items 5 to 7.
Item 17: The DNA-molecule according to any one of items 12 to 15, wherein said RNA-polymerase binding aptamer is an inhibiting RNA-polymerase binding aptamer according to any one of items 8 to 12.
Item 18: A vector for expression comprising a DNA-molecule according to any one of items 12 to 17.
Item 19: A host cell comprising a RNA-molecule according to any one of items 1 to 11, or a DNA molecule according to any one of items 12 to 17, or a vector according to item 18.
Item 20: The use of a RNA-molecule according to any one of items 1 to 11, or a DNA-molecule according to any one of items 12 to 17, or a vector according to item 18 or a host cell according to item 20 for regulating expression of a sequence to be expressed.
Item 21: A method of producing a protein or RNA of interest comprising
   providing a DNA-molecule according to any one of items 12 to 17, or a vector according to item 18, said vector comprising an expression cassette as defined in any one of items 13 to 17,
   introducing said vector into a host cell, and
   culturing said host cell in culture medium under conditions inducing transcription from the promoter of the expression cassette, and
   optionally recovering the protein or RNA of interest from the host cell or culture medium.
Item 22: The method according to item 21, wherein the step of providing the vector comprises the step of inserting a sequence encoding the protein or RNA of interest into the multiple cloning site of the expression cassette comprised in said vector.
Item 23: The method according to item 21 or item 22, wherein the step of culturing said host cell comprises the incubation with an inhibitor of Rho transcription inhibitor, preferably bicyclomycin.
Item 24: A method for in vitro transcription of an RNA of interest comprising the steps of:
   providing a DNA-molecule according to any one of items 12 to 17, or a vector according to claims 1l8, said DNA-molecule comprising a sequence encoding the RNA of interest operatively linked to a promoter and a sequence encoding said RNA-polymerase binding aptamer according to the present invention,
   incubating said DNA-molecule with a RNA-polymerase according to the present invention under conditions allowing transcription from said promoter, and
   optionally recovering the RNA of interest.
Item 25: A method for in vitro expression of a protein of interest comprising the steps of:
   providing a DNA-molecule according any one of items 12 to 17, or a vector according to item 18, said DNA-molecule comprising a sequence encoding the protein of interest operatively linked to a promoter and a sequence encoding said RNA-polymerase binding aptamer,
   incubating said DNA-molecule with components and under conditions allowing transcription from said promoter and translation of the transcript, and
   optionally recovering the protein of interest.
Item 26: The method according to item 25, wherein said components comprise a cell extract from E. coli, and optionally an energy source, a supply of amino acids, cofactors for the transcription and/or translation machinery of the cell extract.
Item 27: The method according to any one of items 24 to 26, wherein said RNA-polymerase binding aptamer is a transcription enhancing RNA-polymerase binding aptamer, preferably as defined in any one of items 4 to 7.

1. The use of a RNA-molecule comprising a RNA-polymerase binding aptamer for regulating expression of a sequence to be expressed, wherein said RNA-polymerase binding aptamer has a length of 15 to 60 nt, preferably 20 to 50 nt, and wherein said RNA-Polymerase binding aptamer binds to a RNA-polymerase with a K_{D} of 50 nM or lower.

2. The use according to claim 1, wherein the RNA-polymerase binding aptamer is used to regulate the expression of the sequence to be expressed in cis.

3. The use according to claim 1, wherein said RNA-polymerase is a prokaryotic RNA-polymerase or eukaryotic RNA-polymerase.

4. The use of claim 1, wherein the RNA-polymerase binding aptamer interacts with the holoenzyme of the RNA-polymerase of Escherichia coli or with RNA-Polymerase II of yeast or Homo sapiens.

5. The use according to claim 4, wherein the RNA-polymerase binding aptamer is a transcription enhancing RNA-polymerase binding aptamer.

6. The use according to claim 5, wherein the transcription enhancing RNA-polymerase binding aptamer increases expression of a sequence to be expressed, wherein the increase of expression is at least 10% as compared to the expression of said sequence to be expressed when not comprising said RNA-polymerase binding aptamer.

7. The use of claim 1, wherein the transcription enhancing RNA-polymerase binding aptamer is encoded by a sequence of SEQ ID NO:139, preferably by a sequence of SEQ ID NO:1 or SEQ ID NO:138; preferably encoded by a sequence having at least 80% identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:72, SEQ ID NO:73, SEQ ID NO:74, SEQ ID NO:75, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, and SEQ ID NO:137.

8. The use of claim 1, wherein the RNA-polymerase binding aptamer is an inhibiting RNA-polymerase binding aptamer.

9. The use according to claim 8, wherein the inhibiting RNA-polymerase binding aptamer reduces expression of a sequence to be expressed, wherein the reduction of expression is at least 30% as compared to the expression of said sequence to be expressed when not comprising said RNA-polymerase binding aptamer, preferably by at least 20%, even more preferably by at least 50%.

10. The use according to claim 8 wherein the inhibiting RNA-polymerase binding aptamer has a C-content of more than 27%, and a G-content of less than 23%.

11. The use according to claim 8, wherein the inhibiting RNA-polymerase binding aptamer is encoded by a sequence selected from the group consisting of SEQ ID NO:84, SEQ ID NO:85, SEQ ID NO:86, and SEQ ID NO:87, preferably the inhibiting RNA-polymerase binding aptamer is encoded by a sequence having at least 80% identity to a sequence selected from the group consisting of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:88, SEQ ID NO:89, SEQ ID NO:90, SEQ ID NO:91, and SEQ ID NO:92.

12. A DNA-molecule comprising a sequence encoding an RNA-polymerase binding aptamer of claim 1.

13. The DNA-molecule according to claim 12, wherein the DNA-molecule comprises an expression cassette, said expression cassette comprising a promoter, said sequence encoding the RNA-polymerase binding aptamer, and a sequence to be expressed or a multiple cloning site for introducing said sequence to be expressed, wherein said promoter, the sequence encoding said RNA-polymerase binding aptamer and said sequence to be expressed are operatively linked, preferably the sequence encoding said RNA-polymerase binding aptamer, and said sequence to be expressed or said multiple cloning site are downstream of said promoter.

14. The DNA-molecule according to claim 13, wherein the sequence to be expressed codes for a protein and the RNA-polymerase binding aptamer is located upstream of the open reading frame.

15. The DNA-molecule according to claim 14, wherein the said expression cassette comprises two or more open reading frames coding for one or more proteins to be expressed, wherein the expression cassette comprises for each open reading frame a sequence encoding the RNA-polymerase binding aptamer operatively linked to said open reading frame, preferably the two or more open reading frames are separated by a sequence encoding said one or more RNA-polymerase binding aptamer and a translation initiation site, both operatively linked to said open reading frame.

16. The DNA-molecule according to claim 12, wherein said RNA-polymerase binding aptamer is a transcription enhancing RNA-polymerase binding aptamer.

17. The DNA-molecule according to claim 12, wherein said RNA-polymerase binding aptamer is an inhibiting RNA-polymerase binding aptamer.

18. A vector for expression comprising a DNA-molecule according to claim 12.

19. A host cell comprising:
(a) a RNA-molecule according to claim 1; or
(b) a DNA molecule comprising a sequence encoding (a); or
(c) a vector comprising (b).

20. The use of
(a) a RNA-molecule according to claim 1; or
(b) a DNA molecule comprising a sequence encoding (a); or
(c) a vector comprising (b); or
(d) a host cell comprising (a)-(c) for regulating expression of a sequence to be expressed.

21. A method of producing a protein or RNA of interest comprising
providing a DNA-molecule according to claim 12, or a vector comprising the DNA-molecule, said vector comprising an expression cassette, said expression cassette comprising a promoter, said sequence encoding the RNA-polymerase binding aptamer, and a sequence to be expressed or a multiple cloning site for introducing said sequence to be expressed, wherein said promoter, the sequence encoding said RNA-polymerase binding aptamer and said sequence to be expressed are operatively linked, preferably the sequence encoding said RNA-polymerase binding aptamer, and said sequence to be expressed or said multiple cloning site are downstream of said promoter,
introducing said vector into a host cell, and
culturing said host cell in culture medium under conditions inducing transcription from the promoter of the expression cassette, and
optionally recovering the protein or RNA of interest from the host cell or culture medium.

22. The method according to claim 21, wherein the step of providing the vector comprises the step of inserting a sequence encoding the protein or RNA of interest into the multiple cloning site of the expression cassette comprised in said vector.

23. The method according to claim 21, wherein the step of culturing said host cell comprises the incubation with an inhibitor of Rho transcription inhibitor, preferably bicyclomycin.

24. A method for in vitro transcription of an RNA of interest comprising the steps of:
providing a DNA-molecule according to claim 12, or a vector comprising the DNA-molecule, said DNA-molecule comprising a sequence encoding the RNA of interest operatively linked to a promoter and a sequence encoding said RNA-polymerase binding aptamer according to the present invention,
incubating said DNA-molecule with a RNA-polymerase according to the present invention under conditions allowing transcription from said promoter, and
optionally recovering the RNA of interest.

25. A method for in vitro expression of a protein of interest comprising the steps of:
providing a DNA-molecule according to claim 12, or a vector comprising the DNA-molecule, said DNA-molecule comprising a sequence encoding the protein of interest operatively linked to a promoter and a sequence encoding said RNA-polymerase binding aptamer,
incubating said DNA-molecule with components and under conditions allowing transcription from said promoter and translation of the transcript, and
optionally recovering the protein of interest.

26. The method according to claim 25, wherein said components comprise a cell extract from E. coli, and optionally an energy source, a supply of amino acids, cofactors for the transcription and/or translation machinery of the cell extract.

27. The method according to claim 24, wherein said RNA-polymerase binding aptamer is a transcription enhancing RNA-polymerase binding aptamer.
